(19) United States
(12) Patent Application Publication
(10) Pub. No.: US 2025/0099399 A1
(43) Pub. Date: Mar. 27, 2025

(54) TRANSDERMAL DELIVERY PATCH FOR TREATING PAIN

(71) Applicant: Teikoku Pharma USA, Inc., San Jose, CA (US)

(72) Inventors: Wan-Ning SONG, San Jose, CA (US); Stephen Shipping HWANG, Palo Alto, CA (US); Bret BERNER, Seattle, WA (US); Jianye WEN, Palo Alto, CA (US); Jutaro SHUDO, Elk Grove, CA (US)

(21) Appl. No.: 18/825,512

(22) Filed: Sep. 5, 2024

Related U.S. Application Data
(60) Provisional application No. 63/651,245, filed on May 23, 2024.
(60) Provisional application No. 63/580,639, filed on Sep. 5, 2023.

(51) Int. Cl. A61K 9/70; A61K 31/4164

(52) U.S. Cl. CPC A61K 9/7061; A61K 9/703; A61K 31/4164

(57) ABSTRACT

The present disclosure relates to the field of transdermal delivery patches containing dexmedetomidine and use of the transdermal delivery patches for treating pain, e.g., post-operative pain.

### 1. BACKGROUND

Pain is the most common reason for physician consultation in the United States. Pain is the unpleasant sensory and emotional experience associated with actual or potential tissue, bone, nerve or cellular damage. Most pain resolves promptly once the painful stimulus is removed and the body has healed, but sometimes pain persists despite removal of the stimulus and apparent healing of the body. It is a major symptom in many medical conditions and can significantly interfere with a person's quality of life and general functioning. Pain can also arise in the absence of any detectable stimulus, damage, or disease. Pain is usually transitory, lasting only until the noxious stimulus is removed or the underlying damage or pathology has healed, but some painful conditions may persist for years.

In the United States, about twenty million surgical procedures are performed under general anesthesia each year. Post-operative pain, pain that occurs after surgery, is a serious and often intractable medical problem. Pain is usually localized within the vicinity of the surgical site. Post-operative pain can have two clinically important aspects, namely resting pain, or pain that occurs when the patient is not moving, and mechanical pain which is exacerbated by movement (e.g., coughing/sneezing, getting out of bed, physiotherapy, etc.). A major problem with post-operative pain management is that the rescue medications currently used have a variety of prominent side effects, e.g., potential for addiction and constipation associated with opioids.

Dexmedetomidine is the S-enantiomer of medetomidine and is an agonist of α₂-adrenergic receptors that is used as a sedative medication in intensive care units and by anesthetists for intubated and non-intubated patients requiring sedation for surgery or short-term procedures. The α₂-adrenergic receptor is a G-protein coupled receptor associated with the Gᵢ heterotrimeric G-protein that includes three highly homologous subtypes, including α₂ₐ, α_{2b} and α_{2c}-adrenergic receptors. Agonists of the α₂-adrenergic receptor are implicated in sedation, muscle relaxation, and analgesia through effects on the central nervous system.

Dexmedetomidine is used in clinical settings as a sedative through intravenous administration and thus, requires close supervision by a health care professional in a hospital setting. Dexmedetomidine is currently employed for sedation of intubated or mechanically ventilated subjects during treatment in an intensive care setting as well as for sedation of non-intubated subjects prior to and/or during non-surgical procedures.

There is therefore a need for improved compositions and methods for treating pain, e.g., post-operative pain.

### 2. SUMMARY

The present disclosure relates to transdermal delivery patches that provide sustained release of dexmedetomidine and their use in the treatment of pain, e.g., post-operative pain.

In one aspect, a transdermal delivery patch comprising a drug layer affixed to a backing layer is provided, wherein:

the drug layer comprises from 1 mg to 3.5 mg dexmedetomidine and a pressure sensitive adhesive,

the drug layer has an adhesive surface suitable for adhesion to a skin surface, and

the transdermal delivery patch is unitary in structure.

In another aspect, the present disclosure provides a transdermal delivery patch system comprising at least two of the transdermal delivery patches described herein adjacent to one another.

In another aspect, the present disclosure provides a kit comprising a plurality of transdermal delivery patches described herein.

In another aspect, the present disclosure provides a method of treating pain (e.g., post-operative pain). In certain embodiments, a method of treating post-operative pain in a subject in need thereof is provided, the method comprising applying one or more transdermal delivery patches described herein or a transdermal delivery patch system described herein to the skin surface of a subject pre-operatively and maintaining the one or more transdermal delivery patches on the skin surface of the subject for at least 72 hours, e.g., 96 hours to 120 hours.

### 3. BRIEF DESCRIPTION OF THE DRAWINGS

These and other features, aspects, and advantages of the present disclosure will become better understood with regard to the following description, and accompanying drawings, where:

FIG. 1 shows a top view and side view of an exemplary transdermal delivery patch of the present disclosure.

FIG. 2 shows a graph of pain intensity (Numerical Rating Scale) over time in the human study described in Example 2. Open circles represent placebo; closed circles represent DMTS 12 cm²; open squares represent DMTS 15 cm²; and open star represents all DMTS treatment groups.

FIG. 3 shows a Kaplan-Meier plot of the time to first use of rescue analgesic medication (morphine, oxycodone, and ibuprofen) in the intention to treat (ITT) analysis population. Open circles represent placebo; closed circles represent DMTS 12 cm²; open squares represent DMTS 15 cm²; and open stars represent all DMTS treatment groups.

FIG. 4 shows a graph depicting mean (standard error—SE) dexmedetomidine plasma concentrations over time for subjects on DMTS treatment (PK/PD) analysis population. Closed circles represent DMTS 12 cm² and open squares represent DMTS 15 cm².

FIG. 5, panels A and B, show the sedation score (Wilson Rating Scale) for the placebo study group (panel A) and DMTS groups (panel B) in the human study described in Example 2. 1=Fully awake and oriented; 2=drowsy; and 3=eyes closed but rousable to command. “EOS” indicates End of Study.

### 4. DETAILED DESCRIPTION

### 1. Definitions

Unless defined otherwise, all technical and scientific terms used herein have the same meaning as commonly understood by one of ordinary skill in the art to which this disclosure applies.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

As used herein, “acute pain” refers to pain lasting up to 30 days, typically in response to some form of tissue injury, such as trauma or surgery. In certain embodiments, “acute pain” is consistent with the International Association for the Study of Pain's definition, which is being of recent onset and limited short duration. Acute pain usually has a temporal (follows immediately after surgery/trauma) and causal (has a known cause) relationship to injury or disease. The intensity of acute pain is greatest at the onset of injury, but with healing pain intensity reduces.

As used herein, “dexmedetomidine” refers to the S-enantiomer of medetomidine:

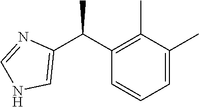

or a pharmaceutically acceptable salt, hydrate or complex of dexmedetomidine. Dexmedetomidine may be in the form of a pharmaceutically acceptable salt including, but not limited to, a mesylate, maleate, fumarate, tartrate, hydrochloride, hydrobromide, p-toluenesulfonate, benzoate, acetate, phosphate or sulfate salt. In certain embodiments, “dexmedetomidine” refers to the hydrochloride salt of dexmedetomidine, i.e., (+)-4-(S)-[1-(2,3-dimethylphenyl)ethyl]-1H-imidazole monohydrochloride.

As used herein, “dosage interval” refers to the period of time the dexmedetomidine transdermal delivery patches described herein are maintained in contact with the skin surface of the subject.

As used herein, “opioid” refers to naturally occurring or synthetic chemical substances that exert pharmacological action by interaction at opioid receptors (i.e., K and 6 opioid receptors). Example opioids include oxycodone, oxymorphone, hydrocodone, hydromorphone, fentanyl, morphine, codeine, methadone, tramadol, and buprenorphine.

As used herein, “non-sedative” refers to an amount of dexmedetomidine that does not cause complete sedation of the subject. Suitable protocols for determining level of sedation may include, but are not limited to, the Ramsay Sedation Scale, the Vancouver Sedative Recovery Scale, the Glasgow Coma Scale modified by Cook and Palma, the Comfort Scale, the New Sheffield Sedation Scale, the Sedation-Agitation Scale, and the Motor Activity Assessment Scale, and the Wilson Sedation Score. In certain embodiments, the level of sedation is evaluated using the Wilson Sedation Scale, details of which are available at the website produced by placing “http://www.” prior to “sedationsolutions.co.uk/training-series6.php” and below:

The Wilson Sedation Score:
1. Fully awake and oriented
2. Drowsy
3. Eyes closed but rousable to command
4. Eyes closed but rousable to mild physical stimulation (earlobe tug)
5. Eyes closed but unrousable to mild physical stimulation

As used herein, “pain” refers to the unpleasant sensory and emotional experience associated with actual or potential tissue damage, or described in terms of such damage (e.g., as defined by the International Association for the Study of Pain). Pain may also involve unpleasant sensory and emotional experience where the damage is not clearly located or cannot be shown to exist. In certain instances, pain includes any sensory experience that causes suffering (physical, psychological, emotional, mental, etc.) in a subject.

As used herein, “post-operative pain” (interchangeable with “post-surgical pain” or “post-incisional pain”) refers to pain arising or resulting from an external trauma such as a cut, puncture, incision, tear, or wound into tissue of an individual that arose from a surgical procedure, whether invasive or non-invasive. Post-operative pain is experienced after a surgical procedure. Post-operative pain includes nociception and the sensation of pain, and can be assessed objectively and subjectively, using pain scores and other methods, e.g., with protocols well-known in the art. Post-operative pain includes allodynia (i.e., pain due to a stimulus that does not normally provoke pain) and hyperalgesia (i.e., increased response to a stimulus that is normally painful), which can in turn, be thermal or mechanical (tactile) in nature. In some embodiments, the post-operative pain is characterized by thermal sensitivity, mechanical sensitivity, and/or resting pain (e.g., persistent pain in the absence of external stimuli). In some embodiments, the post-operative pain includes mechanically induced pain or resting pain. In other embodiments, the post-operative pain includes resting pain.

As used herein, “subject” refers to the person or organism to which the transdermal delivery patch is applied. As such, subjects of the invention may include but are not limited to mammals, e.g., humans and other primates, such as chimpanzees and other apes and monkey species; and the like, where in certain embodiments the subject are humans. The term subject is also meant to include a person or organism of any age, weight, or other physical characteristic, where the subjects may be an adult, an adolescent, a child, an infant, or a newborn.

As used herein, “transdermal” refers to the route of administration where an active agent (i.e., drug) is delivered across the skin (e.g., topical administration) or mucous membrane for systemic distribution.

As such, transdermal delivery patches as described herein are formulated to deliver dexmedetomidine to the subject through one or more of the subcutis, dermis and epidermis, including the stratum corneum, stratum germinativum, stratum spinosum and stratum basale. Accordingly, a transdermal delivery patch may be applied at any convenient location, such as, for example, the arms, legs, thighs, hips, buttocks, abdomen, back, neck, scrotum, vagina, face, forehead, and behind the ear.

As used herein, “treating” or “treatment” refers to a suppression or an amelioration of the symptoms associated with the condition afflicting the subject, where suppression and amelioration are used in a broad sense to refer to at least a reduction in the magnitude of a parameter, e.g., symptom, associated with the condition being treated, such as post-operative pain. As such, treatment also includes situations where the condition is completely inhibited, e.g., prevented from happening, or stopped, e.g., terminated, such that the subject no longer experiences the condition. As such, treatment includes both preventing and managing a condition. In certain embodiments, allodynia is suppressed, ameliorated and/or prevented, and in some embodiments, hyperalgesia is suppressed, ameliorated and/or prevented.

As used herein, “unitary” refers to a single unit or entity of continuous structure. As such, a transdermal delivery patch that is “unitary in structure” refers to a single transdermal delivery patch, in contrast to two or more patches placed adjacent to one another so as to replicate a single larger patch (e.g., a transdermal delivery patch system as described herein).

As used herein, “Cₘₐₓ” refers to the observed maximum (peak) plasma concentration of a specified compound in the subject after administration of a dose of that compound to the subject.

As used herein, “AUC” refers to the average area under the plasma concentration-time curve, which is a measure of exposure to a compound of interest (e.g., dexmedetomidine), and is the integral of the concentration-time curve after a single dose or at steady state. AUC is expressed in units of pg*hr/mL (pg×hr/mL).

As used herein, “AUCₜₐᵤ” refers to the AUC from 0 hours to the end of a dosing interval.

As used herein, “Tₘₐₓ” refers to time to maximum concentration of a specified compound in the subject after administration of a dose of that compound to the subject.

### 2. Transdermal Delivery Patches

In one aspect, the present disclosure provides transdermal delivery patches that provide extended release of the dexmedetomidine formulated within the patch. The transdermal delivery patches described herein, in certain embodiments, release dexmedetomidine continuously over a period of at least 4 days. In certain embodiments, the transdermal delivery patches described herein are useful in the treatment of pain, e.g., post-operative pain when applied to a subject prior to a surgical procedure.

In certain embodiments, a transdermal delivery patch comprising a drug layer affixed to a backing layer is provided, wherein:

the drug layer comprises from 1 mg to 3.5 mg dexmedetomidine and a pressure sensitive adhesive,

the drug layer has an adhesive surface suitable for adhesion to a skin surface, and

the transdermal delivery patch is unitary in structure.

The drug layer is a single layer comprising dexmedetomidine and a pressure sensitive adhesive.

The drug layer is affixed to a backing layer, e.g., by lamination. The surface of the drug layer opposite the backing layer is adhesive and adheres to a skin surface of a subject.

In certain embodiments, the dexmedetomidine is a pharmaceutically acceptable salt of dexmedetomidine. In certain embodiments, the dexmedetomidine is dexmedetomidine hydrochloride.

In certain embodiments, the drug layer comprises from 1 mg to 3.5 mg dexmedetomidine, such as, for example, from 1 mg to 2.75 mg, from 1 mg to 2.5 mg, from 1 mg to 2.25 mg, from 1 mg to 2 mg, from 1 mg to 1.75 mg, from 1 mg to 1.5 mg, from 1 mg to 1.25 mg, from 1.25 mg to 3.5 mg, from 1.25 mg to 3 mg, from 1.25 mg to 2.75 mg, from 1.25 mg to 2.5 mg, from 1.25 mg to 2.25 mg, from 1.25 mg to 2 mg, from 1.25 mg to 1.75 mg, from 1.25 mg to 1.5 mg, from 1.5 mg to 3.5 mg, from 1.5 mg to 3 mg, from 1.5 mg to 2.75 mg, from 1.5 mg to 2.5 mg, from 1.5 mg to 2.25 mg, from 1.5 mg to 2 mg, from 1.5 mg to 1.75 mg, from 1.75 mg to 3.5 mg, from 1.75 mg to 3 mg, from 1.75 mg to 2.75 mg, from 1.75 mg to 2.5 mg, from 1.75 mg to 2.25, from 1.75 mg to 2 mg, from 2 mg to 3.5 mg, from 2 mg to 3 mg, from 2 mg to 2.75 mg, from 2 mg to 2.5 mg, from 2 mg to 2.25 mg, from 2.25 mg to 3.5 mg, from 2.25 mg to 3 mg, from 2.25 mg to 2.75 mg, from 2.25 mg to 2.5 mg, from 2.5 mg to 3.5 mg, from 2.5 mg to 3 mg, from 2.5 mg to 2.75 mg, from 2.5 mg to 3.5 mg, from 2.5 mg to 3.0 mg, from 2.5 mg to 2.75 mg, from 2.75 mg to 3.5 mg, from 2.75 mg to 3 mg, or from 3 mg to 3.5 mg.

In certain embodiments, the drug layer comprises from 1.3 mg to 1.6 mg dexmedetomidine or from 1.4 mg to 1.5 mg dexmedetomidine. In certain embodiments, the drug layer comprises from 1.8 mg to 2.3 mg dexmedetomidine or from 2.0 mg to 2.1 mg dexmedetomidine. In certain embodiments, the drug layer comprises from 2 mg to 2.4 mg dexmedetomidine or from 2.1 to 2.2 mg dexmedetomidine. In certain embodiments, the drug layer comprises from 2.6 mg to 3.2 mg dexmedetomidine or from 2.9 mg to 3 mg dexmedetomidine.

Pressure sensitive adhesives include, but are not limited to, poly-isobutene adhesives, polyisobutylene adhesives, poly-isobutene/polyisobutylene adhesive mixtures, carboxylated polymers, silicon polymers, polyvinyl acetate copolymers (e.g., poly(ethylene-vinyl acetate)), styrenic block copolymers (e.g., polystyrene/polybutadiene), and acrylic or acrylate copolymers, such as carboxylated acrylate copolymers.

In certain embodiments, the pressure sensitive adhesive comprises a silicon polymer. “Silicon polymer,” as used herein, refers to polymerized organosilicon derivatives. In certain embodiments, the silicon polymer comprises apolysiloxane, e.g., polydimethylsiloxane (PDMS), and polydiethylsiloxane, polydipropylsiloxane. In certain embodiments, the silicon polymer is crosslinked to form a silicon resin.

In certain embodiments, the pressure sensitive adhesive comprises a styrenic block copolymer. In certain embodiments, the styrenic block copolymer further comprises polybutadiene, polyisoprene, or polyethylene. In certain embodiments, the styrenic block copolymer is a hydrogenated block copolymer. In certain embodiments, styrenic block copolymer comprises Kraton™ A1535, Kraton™ A1536, or Kraton™ A1537. In certain embodiments, the styrenic block copolymer is an unhydrogenated block copolymer. In certain embodiments, the styrenic block copolymer is Kraton™ D SBS, Kraton™ D SIS, or Kraton™ D SIBS. In certain embodiments, the styrenic block copolymer further comprises maleic anhydride. In certain embodiments, the styrenic block copolymer comprises Kraton™ FG1901 G or Kraton™ FG1924 G.

Where the pressure sensitive adhesive includes polybutene, the polybutene may be saturated polybutene. Alternatively, the polybutene may be unsaturated polybutene. Still further, the polybutene may be a mixture or combination of saturated polybutene and unsaturated polybutene. In certain embodiments, the pressure sensitive adhesive may include a composition that is, or is substantially the same as, the composition of Indopol® L-2, Indopol® L-3, Indopol® L-6, Indopol® L-8, Indopol® L-14, Indopol® H-7, Indopol® H-8, Indopol® H-15, Indopol® H-25, Indopol® H-35, Indopol® H-50, Indopol® H-100, Indopol® H-300, Indopol® H-1200, Indopol® H-1500, Indopol® H-1900, Indopol® H-2100, Indopol® H-6000, Indopol® H-18000, Panalane® L-14E, or Panalane® H-300E. In certain embodiments, the polybutene pressure-sensitive adhesive is Indopol® H-1900. In other embodiments, the polybutene pressure-sensitive adhesive is Panalane® H-300E.

Acrylate copolymers of interest include copolymers of various monomers, such as “soft” monomers, “hard” monomers, or “functional” monomers. The acrylate copolymers can be composed of a copolymer including bipolymer (i.e., made with two monomers), a terpolymer (i.e., made with three monomers), or a tetrapolymer (i.e., made with four monomers), or copolymers having greater numbers of monomers. The acrylate copolymers may be crosslinked or non-crosslinked. The polymers can be crosslinked by known methods to provide the desired polymers. The monomers from of the acrylate copolymers may include at least two or more exemplary components selected from acrylic acids, alkyl acrylates, methacrylates, copolymerizable secondary monomers, and monomers with functional groups.

Monomers (“soft” and “hard” monomers) may be methoxyethyl acrylate, ethyl acrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, acrylonitrile, methoxyethyl acrylate, methoxyethyl methacrylate, and the like. Additional examples of acrylic adhesive monomers are described in Satas, “Acrylic Adhesives,” Handbook of Pressure-Sensitive Adhesive Technology, 2nd ed., pp. 396-456 (D. Satas, ed.), Van Nostrand Reinhold, New York (1989), the disclosure of which is herein incorporated by reference.

In certain embodiments, the pressure sensitive adhesive comprises a polyvinyl acetate copolymer. In certain embodiments, the pressure sensitive adhesive comprises ethylene-vinyl acetate copolymer, vinyl acetate-acrylic acid, polyvinyl chloride acetate, or polyvinylpyrrolidone. In some embodiments, the pressure sensitive adhesive is an acrylate-vinyl acetate copolymer. In some embodiments, the pressure sensitive adhesive may include a composition that is, or is substantially the same as, the composition of Duro-Tak® 87-9301, Duro-Tak® 87-200A, Duro-Tak®87-2353, Duro-Tak®87-2100, Duro-Tak®87-2051, Duro-Tak®87-2052, Duro-Tak®87-2194, Duro-Tak®87-2677, Duro-Tak®87-201A, Duro-Tak®87-2979, Duro-Tak®87-2510, Duro-Tak®87-2516, Duro-Tak®87-387, Duro-Tak®87-4287, Duro-Tak®87-2287, or Duro-Tak®87-2074. The term “substantially the same” as used in this context refers to a composition that is an acrylate-vinyl acetate copolymer in an organic solvent solution.

In certain embodiments, the pressure sensitive adhesive is an acrylate adhesive that is a non-functionalized acrylate, hydroxyl-functionalized acrylate, or an acid functionalized acrylate. For example, the acrylate adhesive may be an acrylic adhesive having one or more —OH functional groups. Where the acrylic adhesive has one or more —OH functional groups, in some instances, the pressure sensitive adhesive may be a composition that is, or is substantially the same as, the composition of Duro-Tak® 87-4287, Duro-Tak® 87-2287, Duro-Tak® 87-2510 or Duro-Tak® 87-2516. The acrylate adhesive may alternatively be an acrylic adhesive having one or more —COOH functional groups. Where the acrylic adhesive has one or more —COOH functional groups, in some instances, the pressure sensitive adhesive may be a composition that is or is substantially the same as, the composition of Duro-Tak® 87-387, Duro-Tak® 87-2979 or Duro-Tak® 87-2353. Still further, the acrylate adhesive may be a non-functionalized acrylic adhesive. Where the acrylic adhesive is non-functionalized, in some instances, the pressure sensitive adhesive may be a composition that is or is substantially the same as, the composition of Duro-Tak® 87-9301.

The amount of pressure sensitive adhesive in the drug layer may vary, for example, the amount of pressure sensitive adhesive ranging from 0.1 mg to 2000 mg, such as, for example, from 0.5 mg to 1500 mg, from 1 to 1000 mg, from 10 to 750 mg, or from 10 mg to 500 mg. As such, in certain embodiments, the amount of pressure sensitive adhesive ranges from 1% to 99% (w/w), such as, for example, from 5% to 95% (w/w), from 10% to 95% (w/w), from 15% to 90% (w/w), or from 20% to 85% (w/w). In other embodiments, the amount of pressure sensitive adhesive in the drug layer is 70% by weight or greater, e.g., 75% by weight or greater, 80% by weight or greater, 85% by weight or greater, 90% by weight or greater, 95% by weight or greater, 97% by weight or greater.

The weight ratio of pressure sensitive adhesive to dexmedetomidine in the drug layer may range between 1:2 and 1:2.5; 1:2.5 and 1:3; 1:3 and 1:3.5 1:3.5 and 1:4; 1:4 and 1:4.5; 1:4.5 and 1:5; 1:5 and 1:10; 1:10 and 1:25; 1:25 and 1:50; 1:50 and 1:75; and 1:75 and 1:99 or a range thereof. For example, the weight ratio of pressure sensitive adhesive to dexmedetomidine in the drug layer may range between 1:1 and 1:5; 1:5 and 1:10; 1:10 and 1:15; 1:15 and 1:25; 1:25 and 1:50; 1:50 and 1:75 or 1:75 and 1:99. Alternatively, the weight ratio of dexmedetomidine to pressure sensitive adhesive in the drug layer may range between 2:1 and 2.5:1; 2.5:1 and 3:1; 3:1 and 3.5:1; 3.5:1 and 4:1; 4:1 and 4.5:1; 4.5:1 and 5:1; 5:1 and 10:1; 10:1 and 25:1; 25:1 and 50:1; 50:1 and 75:1; and 75:1 and 99:1 or a range thereof. For example, the ratio of dexmedetomidine to pressure sensitive adhesive in drug layer may range between 1:1 and 5:1; 5:1 and 10:1; 10:1 and 15:1; 15:1 and 25:1; 25:1 and 50:1; 50:1 and 75:1; or 75:1 and 99:1.

In some embodiments, the drug layer may further include one or more crosslinked hydrophilic polymers. For example, the crosslinked polymer may be an amine-containing hydrophilic polymer. Amine-containing polymers include, but are not limited to, polyethyleneimine, amine-terminated polyethylene oxide, amine-terminated polyethylene/polypropylene oxide, polymers of dimethyl amino ethyl methacrylate, and copolymers of dimethyl amino ethyl methacrylate and vinyl pyrrolidone. In certain embodiments, the crosslinked polymer is crosslinked polyvinylpyrrolidone, such as, for example, PVP-CLM.

The drug layer may contain other additives depending on the adhesive used. For example, materials, such as PVP-CLM, PVP K17, PVP K30, PVP K90, that inhibit drug crystallization, have hygroscopic properties that improve the duration of wear, and improve the physical properties, e.g., cold flow, tack, cohesive strength, of the adhesive.

The amount of crosslinked polymer in the drug layer may vary, for example, the amount of crosslinked polymer ranging from 0.1 mg to 500 mg, such as, for example, from 0.5 mg to 400 mg, from 1 to 300 mg, from 10 to 200 mg, or from 10 mg to 100 mg. As such, in certain embodiments, the amount of crosslinked polymer in the drug layer ranges from 2% to 30% (w/w), such as, for example, from 4% to 30% (w/w), from 5% to 25% (w/w), from 6% to 22.5% (w/w), or from 10% to 20% (w/w). In other embodiments, the amount of crosslinked polymer in the drug layer is 8% by weight or greater, such as, for example, 10% by weight or greater, 12% by weight or greater, 15% by weight or greater, 20% by weight or greater, 25% by weight or greater, or 30% by weight or greater.

In certain embodiments, the drug layer further comprises a permeation enhancer. Permeation enhancers increase the dexmedetomidine solubility, such as, for example, to prevent unwanted crystallization of dexmedetomidine in the drug layer. The permeation enhancer may be incorporated into the drug layer in an amount ranging from 0.01% to 20% (w/w), such as, for example, from 0.05% to 15% (w/w), from 0.1% to 10% (w/w), from 0.5% to 8% (w/w) or from 0.5% to 5% (w/w).

Exemplary permeation enhancers include, but are not limited to, acids including linolic acid, oleic acid, linolenic acid, stearic acid, isostearic acid, levulinic acid, palmitic acid, octanoic acid, decanoic acid, dodecanoic acid, tetradecanoic acid, hexadecanoic acid, octadecanoic acid, N-lauroyl sarcosine, L-pyroglutamic acid, lauric acid, succinic acid, pyruvic acid, glutaric acid, sebacic acid, cyclopentane carboxylic acid, and acylated amino acids. Other permeation enhancers of interest include, but are not limited to, aliphatic alcohols, such as saturated or unsaturated higher alcohols having 12 to 22 carbon atoms (e.g., oleyl alcohol or lauryl alcohol); fatty acid esters, such as isopropyl myristate, diisopropyl adipate, lauryl lactate, propyl laurate, ethyl oleate and isopropyl palmitate; alcohol amines, such as triethanolamine, triethanolamine hydrochloride, and diisopropanolamine; polyhydric alcohol alkyl ethers, such as alkyl ethers of polyhydric alcohols such as glycerol, ethylene glycol, propylene glycol, 1,3-butylene glycol, diglycerol, polyglycerol, diethylene glycol, polyethylene glycol, dipropylene glycol, polypropylene glycol, polypropylene glycolmonolaurate, sorbitan, sorbitol, isosorbide, methyl glucoside, oligosaccharides, and reducing oligosaccharides, where the number of carbon atoms of the alkyl group moiety in the polyhydric alcohol alkyl ethers is preferably 6 to 20; polyoxyethylene alkyl ethers, such as polyoxyethylene alkyl ethers in which the number of carbon atoms of the alkyl group moiety is 6 to 20, and the number of repeating units (e.g., —O—CH₂CH₂—) of the polyoxyethylene chain is 1 to 9, such as but not limited to polyoxyethylene lauryl ether, polyoxyethylene cetyl ether, polyoxyethylene stearyl ether, and polyoxyethylene oleyl ether; glycerides (i.e., fatty acid esters of glycerol), such as glycerol esters of fatty acids having 6 to 18 carbon atoms, where the glycerides may be monoglycerides (i.e., a glycerol molecule covalently bonded to one fatty acid chain through an ester linkage), diglycerides (i.e., a glycerol molecule covalently bonded to two fatty acid chains through ester linkages), triglycerides (i.e., a glycerol molecule covalently bonded to three fatty acid chains through ester linkages), or combinations thereof, where the fatty acid components forming the glycerides include octanoic acid, decanoic acid, dodecanoic acid, tetradecanoic acid, hexadecanoic acid, octadecanoic acid (i.e., stearic acid) and oleic acid; middle-chain fatty acid esters of polyhydric alcohols; lactic acid alkyl esters; dibasic acid alkyl esters; acylated amino acids; pyrrolidone; pyrrolidone derivatives and combinations thereof. Additional permeation enhancers may include lactic acid, tartaric acid, 1,2,6-hexanetriol, benzyl alcohol, lanoline, potassium hydroxide (KOH), tris(hydroxymethyl)aminomethane, glycerol monooleate (GMO), sorbitan monolaurate (SML), sorbitan monooleate (SMO), laureth-4 (LTH), and combinations thereof. In certain embodiments, the permeation enhancer comprises levulinic acid, lauryl lactate, oleic acid, propylene glycolmonolaurate, or a combination thereof.

The drug layer of the transdermal delivery patch is affixed (e.g., by lamination) to a backing layer. In other words, the backing layer is in direct contact with the drug layer. The backing may be flexible so that it can be brought into close contact with the desired application site on the subject. In certain embodiments, the backing is fabricated from a material that does not absorb the dexmedetomidine and does not allow the dexmedetomidine to be leached from the matrix. Backing layers of interest may include, but are not limited to, non-woven fabrics, woven fabrics, films (including sheets), porous bodies, foamed bodies, paper, composite materials obtained by laminating a film on a non-woven fabric or fabric, and combinations thereof.

Non-woven fabrics may include polyolefin resins such as polyethylene and polypropylene; polyester resins such as polyethylene terephthalate, polybutylene terephthalate and polyethylene naphthalate; rayon, polyamide, poly(ester ether), polyurethane, polyacrylic resins, polyvinyl alcohol, styrene-isoprene-styrene copolymers, and styrene-ethylene-propylene-styrene copolymers; and combinations thereof.

Woven fabrics may include cotton, rayon, polyacrylic resins, polyester resins, polyvinyl alcohol, and combinations thereof.

Films may include polyolefin resins such as polyethylene and polypropylene; polyacrylic resins such as polymethyl methacrylate and polyethyl methacrylate; polyester resins such as polyethylene terephthalate, polybutylene terephthalate and polyethylene naphthalate; and besides cellophane, polyvinyl alcohol, ethylene-vinyl alcohol copolymers, polyvinyl chloride, polystyrene, polyurethane, polyacrylonitrile, fluororesins, styrene-isoprene-styrene copolymers, styrene-butadiene rubber, polybutadiene, ethylene-vinyl acetate copolymers, polyamide, and polysulfone; and combinations thereof. In certain embodiments, the film comprises polyethylene and polyethylene terephthalate.

Papers may include impregnated paper, coated paper, wood free paper, Kraft paper, Japanese paper, glassine paper, synthetic paper, and combinations thereof.

The surface of the drug layer opposite the backing layer is adhesive and adheres to a skin surface of a subject. The surface area of the adhesive surface can vary from 5.5 cm² to 20 cm², such as, for example, from 5.5 cm² to 19 cm², from 5.5 cm² to 18 cm², from 5.5 cm² to 17 cm², from 5.5 cm² to 16 cm², from 5.5 cm² to 15 cm², from 5.5 cm² to 14 cm², from 5.5 cm² to 13 cm², from 5.5 cm² to 12 cm², from 5.5 cm² to 11 cm², from 5.5 cm² to 10 cm², from 5.5 cm² to 9 cm², from 5.5 cm² to 8 cm², from 5.5 cm² to 7 cm², from 5.5 cm² to 6 cm², from 6 cm² to 20 cm², from 6 cm² to 19 cm², from 6 cm² to 18 cm², from 6 cm² to 17 cm², from 6 cm² to 16 cm², from 6 cm² to 15 cm², from 6 cm² to 14 cm², from 6 cm² to 13 cm², from 6 cm² to 12 cm², from 6 cm² to 11 cm², from 6 cm² to 10 cm², from 6 cm² to 9 cm², from 6 cm² to 8 cm², from 6 cm² to 7 cm², from 7 cm² to 20 cm², from 7 cm² to 19 cm², from 7 cm² to 18 cm², from 7 cm² to 17 cm², from 7 cm² to 16 cm², from 7 cm² to 15 cm², from 7 cm² to 14 cm², from 7 cm² to 13 cm², from 7 cm² to 12 cm², from 7 cm² to 11 cm², from 7 cm² to 10 cm², from 7 cm² to 9 cm², from 7 cm² to 8 cm², from 8 cm² to 20 cm², from 8 cm² to 19 cm², from 8 cm² to 18 cm², from 8 cm² to 17 cm², from 8 cm² to 16 cm², from 8 cm² to 15 cm², from 8 cm² to 14 cm², from 8 cm² to 13 cm², from 8 cm² to 12 cm², from 8 cm² to 11 cm², from 8 cm² to 10 cm², from 8 cm² to 9 cm², from 9 cm² to 20 cm², from 9 cm² to 19 cm², from 9 cm² to 18 cm², from 9 cm² to 17 cm², from 9 cm² to 16 cm², from 9 cm² to 15 cm², from 9 cm² to 14 cm², from 9 cm² to 13 cm², from 9 cm² to 12 cm², from 9 cm² to 11 cm², from 9 cm² to 10 cm², from 10 cm² to 20 cm², from 10 cm² to 19 cm², from 10 cm² to 18 cm², from cm² to 217 cm², from 10 cm² to 16 cm², from 10 cm² to 15 cm², from 10 cm² to 14 cm², from 10 cm² to 13 cm², from 10 cm² to 12 cm², from 10 cm² to 11 cm², from 11 cm² to 20 cm², from 11 cm² to 19 cm², from 11 cm² to 18 cm², from 11 cm² to 17 cm², from 11 cm² to 16 cm², from 11 cm² to 15 cm², from 11 cm² to 14 cm², from 11 cm² to 13 cm², from 11 cm² to 12 cm², from 12 cm² to 20 cm², from 12 cm² to 19 cm², from 12 cm² to 18 cm², from 12 cm² to 17 cm², from 12 cm² to 16 cm², from 12 cm² to 15 cm², from 12 cm² to 14 cm², from 12 cm² to 13 cm², from 13 cm² to 14 cm², from 14 cm² to 20 cm², from 14 cm² to 19 cm², from 14 cm² to 18 cm², from 14 cm² to 17 cm², from 14 cm² to 16 cm², from 14 cm² to 15 cm², from 15 cm² to 20 cm², from 15 cm² to 19 cm², from 15 cm² to 18 cm², from 15 cm² to 17 cm², 15 cm² to 16 cm², from 16 cm² to 20 cm², from 16 cm² to 19 cm², from 16 cm² to 18 cm², from 16 cm² to 17 cm², from 17 cm² to 20 cm², from 17 cm² to 19 cm², from 17 cm² to 18 cm², from 18 cm² to 20 cm², from 18 cm² to 19 cm² or from 19 cm² to 20 cm².

In certain embodiments, the surface area of the adhesive surface is from 5.5 cm² to 14 cm² and the drug layer comprises from 1 mg to 3.5 mg dexmedetomidine. In certain embodiments, the surface area of the adhesive surface is from 6 cm² to 14 cm² and the drug layer comprises from 1 mg to 3.5 mg dexmedetomidine. In certain embodiments, the surface area of the adhesive surface is from 7 cm² to 14 cm² and the drug layer comprises from 1 mg to 3.5 mg dexmedetomidine. In certain embodiments, the surface area of the adhesive surface is from 8 cm² to 14 cm² and the drug layer comprises from 1 mg to 3.5 mg dexmedetomidine. In certain embodiments, the surface area of the adhesive surface is from 9 cm² to 14 cm² and the drug layer comprises from 1 mg to 3.5 mg dexmedetomidine. In certain embodiments, the surface area of the adhesive surface is from 10 cm² to 14 cm² and the drug layer comprises from 1 mg to 3.5 mg dexmedetomidine. In certain embodiments, the surface area of the adhesive surface is from 11 cm² to 14 cm² and the drug layer comprises from 1 mg to 3.5 mg dexmedetomidine. In certain embodiments, the surface area of the adhesive surface is from 12 cm² to 14 cm² and the drug layer comprises from 1 mg to 3.5 mg dexmedetomidine. In certain embodiments, the surface area of the adhesive surface is from 13 cm² to 14 cm² and the drug layer comprises from 1 mg to 3.5 mg dexmedetomidine.

In certain embodiments, the surface area of the adhesive surface is from 5.5 cm² to 7.5 cm² and the drug layer comprises from 1 mg to 3.5 mg dexmedetomidine. In certain embodiments, the surface area of the adhesive surface is from 5.5 cm² to 7.5 cm² and the drug layer comprises from 1 mg to 2 mg dexmedetomidine. In certain embodiments, the surface area of the adhesive surface is from 5.5 cm² to 7.5 cm² and the drug layer comprises from 1.3 mg to 1.6 mg dexmedetomidine. In certain embodiments, the surface area of the adhesive surface is 6 cm² and the drug layer comprises 1.5 mg dexmedetomidine.

In certain embodiments, the surface area of the adhesive surface is from 7.5 cm² to 9.5 cm² and the drug layer comprises from 1 mg to 3.5 mg dexmedetomidine. In certain embodiments, the surface area of the adhesive surface is from 7.5 cm² to 9.5 cm² and the drug layer comprises from 1.5 mg to 2.5 mg dexmedetomidine. In certain embodiments, the surface area of the adhesive surface is from 7.5 cm² to 9.5 cm² and the drug layer comprises from 1.8 mg to 2.3 mg dexmedetomidine. In certain embodiments, the surface area of the adhesive surface is 8.5 cm² and the drug layer comprises 2.1 mg dexmedetomidine.

In certain embodiments, the surface area of the adhesive surface is from 7 cm² to 10 cm² and the drug layer comprises from 1 mg to 3.5 mg dexmedetomidine. In certain embodiments, the surface area of the adhesive surface is from 7 cm² to 10 cm² and the drug layer comprises from 2 mg to 3 mg dexmedetomidine. In certain embodiments, the surface area of the adhesive surface is from 7 cm² to 10 cm² and the drug layer comprises from 2 mg to 2.4 mg dexmedetomidine. In certain embodiments, the surface area of the adhesive surface is 9 cm² and the drug layer comprises 2.2 mg dexmedetomidine.

In certain embodiments, the surface area of the adhesive surface is from 11 cm² to 13 cm² and the drug layer comprises from 1 mg to 3.5 mg dexmedetomidine. In certain embodiments, the surface area of the adhesive surface is from 11 cm² to 13 cm² and the drug layer comprises from 2.5 mg to 3 mg dexmedetomidine. In certain embodiments, the surface area of the adhesive surface is from 11 cm² to 13 cm² and the drug layer comprises from 2.6 mg to 3.2 mg dexmedetomidine. In certain embodiments, the surface area of the adhesive surface is 12 cm² and the drug layer comprises 2.9 mg dexmedetomidine.

The transdermal delivery patch can be any shape and size so long as the surface area of the adhesive surface is as provided herein. In certain embodiments, the transdermal delivery patch is rectangular. In certain embodiments, the transdermal delivery patch is rectangular with rounded corners. In certain embodiments, the transdermal delivery patch is square. In certain embodiments, the transdermal delivery patch is square with rounded corners. In certain embodiments, the transdermal delivery patch is circular. In certain embodiments, the transdermal delivery patch is oval.

In certain embodiments, the transdermal delivery patch has a length of 0.5 cm to 4 cm and a width of 0.5 cm to 4 cm, such as, for example, a length from 1 cm to 4 cm and a width from 0.5 cm to 4 cm, a length from 1.5 cm to 4 cm and a width from 0.5 cm to 4 cm, a length from 2 cm to 4 cm and a width from 0.5 cm to 4 cm, a length from 2.5 cm to 4 cm and a width from 0.5 cm to 4 cm, a length from 3 cm to 4 cm and a width from 0.5 cm to 4 cm, a length from 3.5 cm to 4 cm and a width from 0.5 cm to 4 cm, a length from 0.5 cm to 4 cm and a width from 1 cm to 4 cm, a length from 0.5 cm to 4 cm and a width from 1.5 cm to 4 cm, a length from 0.5 cm to 4 cm and a width from 2 cm to 4 cm, a length from 0.5 cm to 4 cm and a width from 2.5 cm to 4 cm, a length from 0.5 cm to 4 cm and a width from 3 cm to 4 cm, a length from 0.5 cm to 4 cm and a width from 3.5 cm to 4 cm. In certain embodiments, the transdermal delivery patch has a length of 3.0 cm to 3.5 cm and a width of 2.5 to 3.0 cm. In certain embodiments, the transdermal delivery patch has a length of 3.5 cm to 4 cm and a width of 3 cm to 3.5 cm.

In certain embodiments, the adhesive surface of the transdermal delivery patch is adhered to a release liner. In such embodiments, the drug layer is essentially sandwiched between the backing layer and the release liner. The release liner can be removed manually, thereby exposing the adhesive face of the drug layer of the transdermal delivery patch, which can then be adhered to a skin surface. In certain embodiments, the release liner comprises two distinct segments, each of which can be removed separately. In certain embodiments, the release liner comprises two overlapping segments, each of which can be removed separately.

In certain embodiments, the release liner is a silicon-coated or fluoropolymer-coated release liner.

In certain embodiments, the release liner comprises a polyester sheet. In certain embodiments, the release liner comprises a silicon-coated polyester sheet. In certain embodiments, the release liner comprises a fluoropolymer-coated polyester sheet.

In certain embodiments, the transdermal delivery patch is packaged within a pouch. Any suitable pouch material can be used, e.g., a single layer or multi-layer pouch. Exemplary pouch materials include, but are not limited to, polyesters, polyethylene, foil, and combinations thereof. In certain embodiments, the pouch comprises a polyester, e.g., polyethylene terephthalate. In certain embodiments, the pouch comprises polyethylene. In certain embodiments, the pouch comprises foil. In certain embodiments, the transdermal delivery patch is packaged within a multi-layer pouch.

In one embodiment, a transdermal delivery patch comprises a drug layer affixed to a backing layer, wherein:

the drug layer comprises from 1 mg to 2 mg dexmedetomidine, preferably from 1.3 mg to 1.6 mg dexmedetomidine; a pressure sensitive adhesive comprising a hydroxyl functionalized acrylate polymer; and lauryl lactate;

the drug layer has an adhesive surface suitable for adhesion to a skin surface, wherein the adhesive face has a surface area of 5.5 cm² to 7.5 cm², preferably 6 cm²;

the backing layer comprises polyethylene and polyethylene terephthalate; and

the transdermal delivery patch is unitary in structure.

In one embodiment, a transdermal delivery patch comprises a drug layer affixed to a backing layer, wherein:

the drug layer comprises from 1 mg to 2 mg dexmedetomidine, preferably from 1.3 mg to 1.6 mg dexmedetomidine; a pressure sensitive adhesive comprising a hydroxyl functionalized acrylate polymer; and lauryl lactate;

the drug layer is adhered to a silicon- or fluoropolymer-coated release liner, wherein the release liner, when removed, exposes an adhesive surface of the drug layer suitable for adhesion to a skin surface, wherein the adhesive face has a surface area of 5.5 cm² to 7.5 cm², preferably 6 cm²;

the backing layer comprises polyethylene and polyethylene terephthalate; and

the transdermal delivery patch is unitary in structure.

In one embodiment, a transdermal delivery patch comprises a drug layer affixed to a backing layer, wherein:

the drug layer comprises from 1.5 mg to 2.5 mg dexmedetomidine, preferably from 1.8 mg to 2.3 mg dexmedetomidine; a pressure sensitive adhesive comprising a hydroxyl functionalized acrylate polymer; and lauryl lactate;

the drug layer has an adhesive surface suitable for adhesion to a skin surface, wherein the adhesive face has a surface area of 7.5 cm² to 9.5 cm², preferably 8.5 cm²;

the backing layer comprises polyethylene and polyethylene terephthalate; and the transdermal delivery patch is unitary in structure.

In one embodiment, a transdermal delivery patch comprises a drug layer affixed to a backing layer, wherein:

the drug layer comprises from 1.5 mg to 2.5 mg dexmedetomidine, preferably from 1.8 mg to 2.3 mg dexmedetomidine; a pressure sensitive adhesive comprising a hydroxyl functionalized acrylate polymer; and lauryl lactate;

the drug layer is adhered to a silicon- or fluoropolymer-coated release liner, wherein the release liner, when removed, exposes an adhesive surface of the drug layer suitable for adhesion to a skin surface, wherein the adhesive face has a surface area of 7.5 cm² to 9.5 cm², preferably 8.5 cm²;

the backing layer comprises polyethylene and polyethylene terephthalate; and

the transdermal delivery patch is unitary in structure.

In one embodiment, a transdermal delivery patch comprises a drug layer affixed to a backing layer, wherein:

the drug layer comprises from 2 mg to 3 mg dexmedetomidine, preferably from 2 mg to 2.4 mg dexmedetomidine; a pressure sensitive adhesive comprising a hydroxyl functionalized acrylate polymer; and lauryl lactate;

the drug layer has an adhesive surface suitable for adhesion to a skin surface, wherein the adhesive face has a surface area of 7 cm² to 10 cm², preferably 9 cm²;

the backing layer comprises polyethylene and polyethylene terephthalate; and

the transdermal delivery patch is unitary in structure.

In one embodiment, a transdermal delivery patch comprises a drug layer affixed to a backing layer, wherein:

the drug layer comprises from 2 mg to 3 mg dexmedetomidine, preferably from 2 mg to 2.4 mg dexmedetomidine; a pressure sensitive adhesive comprising a hydroxyl functionalized acrylate polymer; and lauryl lactate;

the drug layer is adhered to a silicon- or fluoropolymer-coated release liner, wherein the release liner, when removed, exposes an adhesive surface of the drug layer suitable for adhesion to a skin surface, wherein the adhesive face has a surface area of 7 cm² to 10 cm², preferably 9 cm²;

the backing layer comprises polyethylene and polyethylene terephthalate; and

the transdermal delivery patch is unitary in structure.

In one embodiment, a transdermal delivery patch comprises a drug layer affixed to a backing layer, wherein:

the drug layer comprises from 2.5 mg to 3.5 mg dexmedetomidine, preferably from 2.6 mg to 3.2 mg dexmedetomidine; a pressure sensitive adhesive comprising a hydroxyl functionalized acrylate polymer; and lauryl lactate;

the drug layer has an adhesive surface suitable for adhesion to a skin surface, wherein the adhesive face has a surface area of 11 cm² to 13 cm², preferably 12 cm²;

the backing layer comprises polyethylene and polyethylene terephthalate; and

the transdermal delivery patch is unitary in structure.

In one embodiment, a transdermal delivery patch comprises a drug layer affixed to a backing layer, wherein:

the drug layer comprises from 2.5 mg to 3.5 mg dexmedetomidine, preferably from 2.6 mg to 3.2 mg dexmedetomidine; a pressure sensitive adhesive comprising a hydroxyl functionalized acrylate polymer; and lauryl lactate;

the drug layer is adhered to a silicon- or fluoropolymer-coated release liner, wherein the release liner, when removed, exposes an adhesive surface of the drug layer suitable for adhesion to a skin surface, wherein the adhesive face has a surface area of 11 cm² to 13 cm², preferably 12 cm²;

the backing layer comprises polyethylene and polyethylene terephthalate; and

the transdermal delivery patch is unitary in structure.

The present disclosure also provides a transdermal delivery patch system comprising at least two of the transdermal delivery patches described herein adjacent to one another. “Adjacent,” as used herein, means that the transdermal delivery patches are next to one another or adjoining one another. In certain embodiments, a transdermal delivery patch system comprises two, three, four, or five or more transdermal delivery patches described herein. In certain embodiments, a transdermal delivery patch system comprises two transdermal delivery patches described herein. In certain embodiments, the transdermal delivery patches are adjacent on the skin surface of a subject. In certain embodiments, the transdermal delivery patches are adjacent on a release liner.

The present disclosure also provides a kit comprising a plurality of transdermal delivery patches described herein, such as, for example, at least 2, at least 3, at least 5, at least 10, at least 20, at least 30, at least 40, or at least 50. In certain embodiments, each transdermal delivery patch within the kit is packaged within a pouch as described herein. In certain embodiments, the kit comprises a box for enclosing one or more transdermal delivery patches. In certain embodiments, the kit comprises instructions for administering the transdermal delivery patches.

### 3. Methods of Treatment

In one aspect of the present disclosure, a method of treating pain in a subject in need thereof is provided, the method comprising applying one or more of the transdermal delivery patches described herein, or a transdermal delivery system described herein, to the skin surface of a subject and maintaining the one or more transdermal delivery patches on the on the skin surface of the subject for at least 48 hours, e.g., at least 72 hours, at least 84 hours, at least 96 hours, or up to 120 hours.

Exemplary pain indications include, but are not limited to, neuralgia, trigeminal neuralgia, myalgia, hyperalgesia, hyperpathia, neuritis, neuropathy, neuropathic pain, idiopathic pain, acute pain, sympathetically mediated pain, complex regional pain, chronic pain, cancer pain, post-operative pain, post-herpetic neuralgia, irritable bowel syndrome and other visceral pain, radiation pain, diabetic neuropathy, pain associated with muscle spasticity, complex regional pain syndrome (CRPS), sympathetically maintained pain, headache pain, migraine headaches, allodynic pain, inflammatory pain, pain associated with arthritis, gastrointestinal pain, irritable bowel syndrome (IBS), and Crohn's disease. In certain embodiments, a subject's pain is a symptom of an underlying physiological abnormality, such as cancer, arthritis, viral infection such as herpes zoster, or physical trauma such as a burn, injury or surgery, chemotherapy-induced pain, painful chronic chemotherapy induced peripheral neuropathy (CCIPN), or radiation therapy pain.

In certain embodiments, the pain is acute pain. In such embodiments, a method of treating acute pain in a subject comprises applying one or more of the transdermal delivery patches described herein, or a transdermal delivery patch system described herein, to the skin surface of the subject and maintaining the one or more transdermal delivery patches on the skin surface of the subject for at least 48 hours, e.g., at least 72 hours, at least 84 hours, at least 96 hours, or up to 120 hours.

In certain embodiments, the acute pain is part of a multimodal analgesia (MMA) regimen.

In certain embodiments, the acute pain is post-operative acute pain. In certain amendments, the post-operative acute pain is part of a multimodal analgesia (MMA) regimen.

In certain embodiments, the pain is chronic pain. In such embodiments, a method of treating chronic pain in a subject comprises applying one or more of the transdermal delivery patches described herein, or a transdermal delivery patch system described herein, to the skin surface of the subject and maintaining the one or more transdermal delivery patches on the skin surface of the subject for at least 48 hours, e.g., at least 72 hours, at least 84 hours, at least 96 hours, or up to 120 hours.

In certain embodiments, the pain is post-operative pain. In such embodiments, a method of treating post-operative pain in a subject comprises applying one or more of the transdermal delivery patches described herein, or a transdermal delivery patch system described herein, to the skin surface of the subject pre-operatively and maintaining the one or more transdermal delivery patches on the skin surface of the subject for at least 48 hours, e.g., at least 72 hours, at least 84 hours, at least 96 hours, or up to 120 hours.

In certain embodiments, the method comprises applying one transdermal delivery patch to the skin surface of the subject. In certain embodiments, the method comprises applying two or more transdermal delivery patches to the skin surface of the subject, such as, for example, two transdermal delivery patches, three transdermal delivery patches, or four transdermal delivery patches. In certain embodiments, the method comprises applying a transdermal delivery system to the skin surface of the subject.

The adhesive surface of the one or more transdermal delivery patches is applied to the skin surface, particularly a non-hairy surface to maximize adhesion. Suitable skin surfaces include the arms, legs, buttocks, abdomen, back, neck, scrotum, vagina, face, and behind the ear. In certain embodiments, the one or more transdermal delivery patches is applied to the arm of the subject, e.g., the upper arm. In other embodiments, the one or more transdermal delivery patches is/are applied to the back of the subject, e.g., the upper back, such as below one or both shoulders or in the middle of the upper back.

In embodiments where the one or more transdermal delivery patches include a release liner covering the adhesive surface, the method further comprises removing the release liner to expose the adhesive surface prior to applying the one or more transdermal delivery patches to the skin surface of the subject.

In certain embodiments, the one or more transdermal delivery patches or transdermal delivery patch systems may be applied to the skin of the subject after an injury, e.g., tissue injury or surgery. In certain other embodiments, the one or more transdermal delivery patches or transdermal delivery patch systems may be applied to the skin of the subject before an injury, e.g., tissue injury or surgery.

In certain embodiments, the one or more transdermal delivery patches or transdermal delivery patch system may be applied to the skin surface of the subject immediately before surgery, e.g., 0 hours pre-operatively or 0 to 1 hour pre-operatively.

In certain other embodiments, the one or more transdermal delivery patches or transdermal delivery patch system may be applied to the skin surface of the subject at least 1 hour pre-operatively, such as, for example, at least 2 hours pre-operatively, at least 3 hours pre-operatively, at least 4 hours pre-operatively, at least 5 hours pre-operatively, at least 6 hours pre-operatively, at least 7 hours pre-operatively, at least 8 hours pre-operatively, at least 9 hours pre-operatively, at least 10 hours pre-operatively, at least 11 hours pre-operatively, at least 12 hours pre-operatively, at least 13 hours pre-operatively, at least 14 hours pre-operatively, at least 15 hours pre-operatively, at least 16 hours pre-operatively, at least 17 hours pre-operatively, at least 18 hours pre-operatively, at least 19 hours pre-operatively, at least 20 hours pre-operatively, at least 21 hours pre-operatively, at least 22 hours pre-operatively, at least 23 hours pre-operatively, or at least 24 hours pre-operatively.

In certain embodiments, the one or more transdermal delivery patches or transdermal delivery patch system may be applied to the skin surface of the from 0 to 24 hours pre-operatively, such as, for example, 0 to 4 hours pre-operatively, 0 to 2 hours pre-operatively, or 0 to 1 hour pre-operatively.

In certain embodiments, the one or more transdermal delivery patches or transdermal delivery patch system may be applied to the skin surface of the from 1 to 24 hours pre-operatively, such as, for example, 1 to 5 hours pre-operatively, 1 to 3 hours pre-operatively, or 1 to 2 hours pre-operatively.

In certain embodiments, the one or more transdermal delivery patches or transdermal delivery patch system may be applied to the skin surface of the from 2 to 24 hours pre-operatively, such as, for example, from 2 to 23 hours pre-operatively, from 2 to 22 hours pre-operatively, from 2 to 21 hours pre-operatively, from 2 to 20 hours pre-operatively, from 2 to 19 hours pre-operatively, from 2 to 18 hours pre-operatively, from 2 to 17 hours pre-operatively, from 2 to 16 hours pre-operatively, from 2 to 15 hours pre-operatively, from 2 to 14 hours pre-operatively, from 2 to 13 hours pre-operatively, from 2 to 12 hours pre-operatively, from 2 to 11 hours pre-operatively, from 2 to 10 hours pre-operatively, from 2 to 9 hours pre-operatively, from 2 to 8 hours pre-operatively, from 2 to 7 hours pre-operatively, from 2 to 6 hours pre-operatively, from 2 to 5 hours pre-operatively, from 2 to 4 hours pre-operatively, or from 2 to 3 hours pre-operatively.

In certain embodiments, the one or more transdermal delivery patches or transdermal delivery patch system may be applied to the skin surface of the from 3 to 24 hours pre-operatively, such as, for example, from 3 to 23 hours pre-operatively, from 3 to 22 hours pre-operatively, from 3 to 21 hours pre-operatively, from 3 to 20 hours pre-operatively, from 3 to 19 hours pre-operatively, from 3 to 18 hours pre-operatively, from 3 to 17 hours pre-operatively, from 3 to 16 hours pre-operatively, from 3 to 15 hours pre-operatively, from 3 to 14 hours pre-operatively, from 3 to 13 hours pre-operatively, from 3 to 12 hours pre-operatively, from 3 to 11 hours pre-operatively, from 3 to 10 hours pre-operatively, from 3 to 9 hours pre-operatively, from 3 to 8 hours pre-operatively, from 3 to 7 hours pre-operatively, from 3 to 6 hours pre-operatively, from 3 to 5 hours pre-operatively, or from 3 to 4 hours pre-operatively.

In certain embodiments, the one or more transdermal delivery patches or transdermal delivery patch system may be applied to the skin surface of the from 4 to 24 hours pre-operatively, such as, for example, from 4 to 23 hours pre-operatively, from 4 to 22 hours pre-operatively, from 4 to 21 hours pre-operatively, from 4 to 20 hours pre-operatively, from 4 to 19 hours pre-operatively, from 4 to 18 hours pre-operatively, from 4 to 17 hours pre-operatively, from 4 to 16 hours pre-operatively, from 4 to 15 hours pre-operatively, from 4 to 14 hours pre-operatively, from 4 to 13 hours pre-operatively, from 4 to 12 hours pre-operatively, from 4 to 11 hours pre-operatively, from 4 to 10 hours pre-operatively, from 4 to 9 hours pre-operatively, from 4 to 8 hours pre-operatively, from 4 to 7 hours pre-operatively, from 4 to 6 hours pre-operatively, or from 4 to 5 hours pre-operatively.

In certain embodiments, the one or more transdermal delivery patches or transdermal delivery patch system may be applied to the skin surface of the from 5 to 24 hours pre-operatively, such as, for example, from 5 to 23 hours pre-operatively, from 5 to 22 hours pre-operatively, from 5 to 21 hours pre-operatively, from 5 to 20 hours pre-operatively, from 5 to 19 hours pre-operatively, from 5 to 18 hours pre-operatively, from 5 to 17 hours pre-operatively, from 5 to 16 hours pre-operatively, from 5 to 15 hours pre-operatively, from 5 to 14 hours pre-operatively, from 5 to 13 hours pre-operatively, from 5 to 12 hours pre-operatively, from 5 to 11 hours pre-operatively, from 5 to 10 hours pre-operatively, from 5 to 9 hours pre-operatively, from 5 to 8 hours pre-operatively, from 5 to 7 hours pre-operatively, or from 5 to 6 hours pre-operatively.

In certain embodiments, the one or more transdermal delivery patches or transdermal delivery patch system is applied to the skin surface of the subject from 6 to 24 hours pre-operatively, such as, for example, from 6 to 23 hours pre-operatively, from 6 to 22 hours pre-operatively, from 6 to 21 hours pre-operatively, from 6 to 20 hours pre-operatively, from 6 to 19 hours pre-operatively, from 6 to 18 hours pre-operatively, from 6 to 17 hours pre-operatively, from 6 to 17 hours pre-operatively, from 6 to 16 hours pre-operatively, from 6 to 15 hours pre-operatively, from 6 to 14 hours pre-operatively, from 6 to 13 hours pre-operatively, from 6 to 12 hours pre-operatively, from 6 to 11 hours pre-operatively, from 6 to 10 hours pre-operatively from 6 to 9 hours pre-operatively, from 6 to 8 hours pre-operatively, or from 6 to 7 hours pre-operatively.

In certain embodiments, the one or more transdermal delivery patches or transdermal delivery patch system is applied to the skin surface of the subject from 6 to 8 hours pre-operatively.

In certain embodiments, the one or more transdermal delivery patches or transdermal delivery patch system may be applied to the skin surface of the from 7 to 24 hours pre-operatively, such as, for example, from 7 to 23 hours pre-operatively, from 7 to 22 hours pre-operatively, from 7 to 21 hours pre-operatively, from 7 to 20 hours pre-operatively, from 7 to 19 hours pre-operatively, from 7 to 18 hours pre-operatively, from 7 to 17 hours pre-operatively, from 7 to 16 hours pre-operatively, from 7 to 15 hours pre-operatively, from 7 to 14 hours pre-operatively, from 7 to 13 hours pre-operatively, from 7 to 12 hours pre-operatively, from 7 to 11 hours pre-operatively, from 7 to 10 hours pre-operatively, from 7 to 9 hours pre-operatively, or from 7 to 8 hours pre-operatively.

In certain embodiments, the one or more transdermal delivery patches or transdermal delivery patch system may be applied to the skin surface of the from 8 to 24 hours pre-operatively, such as, for example, from 8 to 23 hours pre-operatively, from 8 to 22 hours pre-operatively, from 8 to 21 hours pre-operatively, from 8 to 20 hours pre-operatively, from 8 to 19 hours pre-operatively, from 8 to 18 hours pre-operatively, from 8 to 17 hours pre-operatively, from 8 to 16 hours pre-operatively, from 8 to 15 hours pre-operatively, from 8 to 14 hours pre-operatively, from 8 to 13 hours pre-operatively, from 8 to 12 hours pre-operatively, from 8 to 11 hours pre-operatively, from 8 to 10 hours pre-operatively, or from 8 to 9 hours pre-operatively.

In certain embodiments, the one or more transdermal delivery patches or transdermal delivery patch system may be applied to the skin surface of the from 9 to 24 hours pre-operatively, such as, for example, from 9 to 23 hours pre-operatively, from 9 to 22 hours pre-operatively, from 9 to 21 hours pre-operatively, from 9 to 20 hours pre-operatively, from 9 to 19 hours pre-operatively, from 9 to 18 hours pre-operatively, from 9 to 17 hours pre-operatively, from 9 to 16 hours pre-operatively, from 9 to 15 hours pre-operatively, from 9 to 14 hours pre-operatively, from 9 to 13 hours pre-operatively, from 9 to 12 hours pre-operatively, from 9 to 11 hours pre-operatively, or from 9 to 10 hours pre-operatively.

In certain embodiments, the one or more transdermal delivery patches or transdermal delivery patch system may be applied to the skin surface of the from 10 to 24 hours pre-operatively, such as, for example, from 10 to 23 hours pre-operatively, from 10 to 22 hours pre-operatively, from 10 to 21 hours pre-operatively, from 10 to 20 hours pre-operatively, from 10 to 19 hours pre-operatively, from 10 to 18 hours pre-operatively, from 10 to 17 hours pre-operatively, from 10 to 16 hours pre-operatively, from 10 to 15 hours pre-operatively, from 10 to 14 hours pre-operatively, from 10 to 13 hours pre-operatively, from 10 to 12 hours pre-operatively, or from 10 to 11 hours pre-operatively.

In certain embodiments, the one or more transdermal delivery patches or transdermal delivery patch system may be applied to the skin surface of the from 11 to 24 hours pre-operatively, such as, for example, from 11 to 23 hours pre-operatively, from 11 to 22 hours pre-operatively, from 11 to 21 hours pre-operatively, from 11 to 20 hours pre-operatively, from 11 to 19 hours pre-operatively, from 11 to 18 hours pre-operatively, from 11 to 17 hours pre-operatively, from 11 to 16 hours pre-operatively, from 11 to 15 hours pre-operatively, from 11 to 14 hours pre-operatively, from 11 to 13 hours pre-operatively, or from 11 to 12 hours pre-operatively.

In certain embodiments, the one or more transdermal delivery patches or transdermal delivery patch system may be applied to the skin surface of the from 12 to 24 hours pre-operatively, such as, for example, from 12 to 23 hours pre-operatively, from 12 to 22 hours pre-operatively, from 12 to 21 hours pre-operatively, from 12 to 20 hours pre-operatively, from 12 to 19 hours pre-operatively, from 12 to 18 hours pre-operatively, from 12 to 17 hours pre-operatively, from 12 to 16 hours pre-operatively, from 12 to 15 hours pre-operatively, from 12 to 14 hours pre-operatively, or from 12 to 13 hours pre-operatively.

In certain embodiments, the one or more transdermal delivery patches or transdermal delivery patch system may be applied to the skin surface of the from 13 to 24 hours pre-operatively, such as, for example, from 13 to 23 hours pre-operatively, from 13 to 22 hours pre-operatively, from 13 to 21 hours pre-operatively, from 13 to 20 hours pre-operatively, from 13 to 19 hours pre-operatively, from 13 to 18 hours pre-operatively, from 13 to 17 hours pre-operatively, from 13 to 16 hours pre-operatively, from 13 to 15 hours pre-operatively, or from 13 to 14 hours pre-operatively.

In certain embodiments, the one or more transdermal delivery patches or transdermal delivery patch system may be applied to the skin surface of the from 14 to 24 hours pre-operatively, such as, for example, from 14 to 23 hours pre-operatively, from 14 to 22 hours pre-operatively, from 14 to 21 hours pre-operatively, from 14 to 20 hours pre-operatively, from 14 to 19 hours pre-operatively, from 14 to 18 hours pre-operatively, from 14 to 17 hours pre-operatively, from 14 to 16 hours pre-operatively, or from 14 to 15 hours pre-operatively.

In certain embodiments, the one or more transdermal delivery patches or transdermal delivery patch system may be applied to the skin surface of the from 15 to 24 hours pre-operatively, such as, for example, from 15 to 23 hours pre-operatively, from 15 to 22 hours pre-operatively, from 15 to 21 hours pre-operatively, from 15 to 20 hours pre-operatively, from 15 to 19 hours pre-operatively, from 15 to 18 hours pre-operatively, from 15 to 17 hours pre-operatively, or from 15 to 16 hours pre-operatively.

In certain embodiments, the one or more transdermal delivery patches or transdermal delivery patch system may be applied to the skin surface of the from 16 to 24 hours pre-operatively, such as, for example, from 16 to 23 hours pre-operatively, from 16 to 22 hours pre-operatively, from 16 to 21 hours pre-operatively, from 16 to 20 hours pre-operatively, from 16 to 19 hours pre-operatively, from 16 to 18 hours pre-operatively, or from 16 to 17 hours pre-operatively.

In certain embodiments, the one or more transdermal delivery patches or transdermal delivery patch system may be applied to the skin surface of the from 17 to 24 hours pre-operatively, such as, for example, from 17 to 23 hours pre-operatively, from 17 to 22 hours pre-operatively, from 17 to 21 hours pre-operatively, from 17 to 20 hours pre-operatively, from 17 to 19 hours pre-operatively, or from 17 to 18 hours pre-operatively.

In certain embodiments, the one or more transdermal delivery patches or transdermal delivery patch system may be applied to the skin surface of the from 18 to 24 hours pre-operatively, such as, for example, from 18 to 23 hours pre-operatively, from 18 to 22 hours pre-operatively, from 18 to 21 hours pre-operatively, from 18 to 20 hours pre-operatively, or from 18 to 19 hours pre-operatively.

In certain embodiments, the one or more transdermal delivery patches or transdermal delivery patch system may be applied to the skin surface of the from 19 to 24 hours pre-operatively, such as, for example, from 19 to 23 hours pre-operatively, from 19 to 22 hours pre-operatively, from 19 to 21 hours pre-operatively, or from 19 to 20 hours pre-operatively.

In certain embodiments, the one or more transdermal delivery patches or transdermal delivery patch system may be applied to the skin surface of the from 20 to 24 hours pre-operatively, such as, for example, from 20 to 23 hours pre-operatively, from 20 to 22 hours pre-operatively, or from 20 to 21 hours pre-operatively.

In certain embodiments, the one or more transdermal delivery patches or transdermal delivery patch system may be applied to the skin surface of the from 21 to 24 hours pre-operatively, such as, for example, from 21 to 23 hours pre-operatively or from 21 to 22 hours pre-operatively.

In certain embodiments, the one or more transdermal delivery patches or transdermal delivery patch system may be applied to the skin surface of the from 22 to 24 hours pre-operatively, such as, for example, from 22 to 23 hours pre-operatively.

In certain embodiments, the one or more transdermal delivery patches or transdermal delivery patch system are maintained in contact with the skin surface of the subject for at least 48 hours after surgery, e.g., at least 56 hours, at least 64 hours, at least 72 hours, at least 84 hours, or at least 96 hours after surgery.

In certain embodiments, the one or more transdermal delivery patches or transdermal delivery patch system are applied to the skin surface of the subject immediately before surgery, e.g., 0 hours pre-operatively or 0 to 1 hour pre-operatively and maintained in contact with the skin surface of the subject for at least 48 hours after surgery, e.g., at least 72 hours, at least 84 hours, or at least 96 hours after surgery.

In certain embodiments, the one or more transdermal delivery patches or transdermal delivery patch system are applied to the skin surface of the subject at least 1 hour pre-operatively, e.g., 1 to 2 hours, 1 to 3 hours, 1 to 4 hours, 1 to 6 hours, or 1 to 8 hours pre-operatively, and maintained in contact with the skin surface of the subject for at least 48 hours after surgery, e.g., at least 72 hours, at least 84 hours, or at least 96 hours after surgery.

In certain embodiments, the one or more transdermal delivery patches or transdermal delivery patch system are applied to the skin surface of the subject at least 2 hours pre-operatively, e.g., from 2 to 4 hours, 2 to 6 hours, or 2 to 8 hours pre-operatively, preferably 2 to 8 hours pre-operatively, and maintained in contact with the skin surface of the subject for at least 48 hours after surgery, e.g., at least 72 hours, at least 84 hours, or at least 96 hours after surgery.

In certain embodiments, the one or more transdermal delivery patches or transdermal delivery patch system are applied to the skin surface of the subject at least 4 hours pre-operatively, e.g., from 4 to 6 hours or from 4 to 8 hours pre-operatively, preferably 4 to 8 hours pre-operatively, and maintained in contact with the skin surface of the subject for at least 48 hours after surgery, e.g., at least 72 hours, at least 84 hours, or at least 96 hours after surgery.

In certain embodiments, the one or more transdermal delivery patches or transdermal delivery patch system are applied to the skin surface of the subject at least 6 hours pre-operatively, e.g., from 6 to 8 hours pre-operatively, and maintained in contact with the skin surface of the subject for at least 48 hours after surgery, e.g., at least 72 hours, at least 84 hours, or at least 96 hours after surgery.

In certain embodiments, the one or more transdermal delivery patches or transdermal delivery patch system are maintained in contact with the skin surface of the subject in a manner sufficient to treat post-operative pain in the subject for at least 48 hours after surgery, e.g., at least 56 hours, at least 64 hours, at least 72 hours, at least 84 hours, or at least 96 hours after surgery.

In certain embodiments, the one or more transdermal delivery patches or transdermal delivery patch system are applied to the skin surface of the subject at least 2 hours pre-operatively, e.g., from 2 to 4 hours, 2 to 6 hours, or 2 to 8 hours pre-operatively, preferably 2 to 8 hours pre-operatively, and maintained in contact with the skin surface of the subject in a manner sufficient to treat post-operative pain in the subject for at least 48 hours after surgery, e.g., at least 72 hours, at least 84 hours, or at least 96 hours after surgery.

In certain embodiments, the one or more transdermal delivery patches or transdermal delivery patch system are applied to the skin surface of the subject at least 4 hours pre-operatively, e.g., 4 to 6 hours or 4 to 8 hours pre-operatively, preferably 4 to 8 hours pre-operatively, and maintained in contact with the skin surface of the subject in a manner sufficient to treat post-operative pain in the subject for at least 48 hours after surgery, e.g., at least 72 hours, at least 84 hours, or at least 96 hours after surgery.

In certain embodiments, the one or more transdermal delivery patches or transdermal delivery patch system are applied to the skin surface of the subject at least 6 hours pre-operatively, e.g., from 6 to 8 hours pre-operatively, and maintained in contact with the skin surface of the subject in a manner sufficient to treat post-operative pain in the subject for at least 48 hours after surgery, e.g., at least 72 hours, at least 84 hours, or at least 96 hours after surgery.

The operation or surgical procedure can vary. In certain embodiments, the surgical procedure is a bony surgical procedure or a soft tissue surgery, including, but are not limited to, median sternotomies, laparoscopies, mastectomies, arthroplasties, osteotomies, cancer surgeries, knee surgeries, shoulder surgeries, bunionectomies, total knee arthroplasties, hernia repairs, open cholecystectomies, total hip arthroplasties, or arthroscopic procedures for the shoulder and knee. In certain embodiments, the surgical procedure is a bony surgery. In certain embodiments, the surgical procedure is a bunionectomy.

In certain embodiments, the surgical tissue is a soft tissue surgery such as, for example, an abdominoplasty, breast augmentation or reduction, unilateral inguinal hernia repair, open ventral hernia repair, abdominal hysterectomy, or colectomy. In certain embodiments, the soft tissue surgery is an abdominoplasty.

In certain embodiments, the surgical procedure utilizes general anesthesia to sedate the subject during the surgery. In certain embodiments, general anesthesia is administered to the subject peri-operatively. Exemplary general anesthetics include, but are not limited to, propofol, etomidate, ketamine, barbiturates (e.g., amobarbital, methohexital, thiamylal, and thiopental), benzodiazepines (e.g., diazepam, lorazepam and midazolam), opioid general anesthetics (e.g., alfentanil, fentanyl, remifentanil, sufentanil, buprenorphine, butorphanol, diacetyl morphine, hydromorphone, levorphanol, meperidine, methadone, morphine, nalbuphine, oxycodone, oxymorphone, pentazocine), inhaled general anesthetic agents (e.g., desflurane, enflurane, halothane, isoflurane, methoxyflurane, nitrous oxide, sevoflurane, xenon, and rocuronium). In certain embodiments, the general anesthesia is selected from propofol, rocuronium, fentanyl, and a combination thereof.

In certain embodiments, the one or more transdermal delivery patches or transdermal delivery patch system are maintained in contact with the skin surface of the subject in a manner sufficient to provide an area under the plasma dexmedetomidine concentration curve (AUC₅₋₉₆) of greater than 15,000 pg*hr/mL, e.g., greater than 15,500 pg*hr/mL, greater than 16,000 pg*hr/mL, greater than 16,500 pg*hr/mL, greater than 17,000 pg*hr/mL, greater than 17,500 pg*hr/mL, greater than 18,000 pg*hr/mL, greater than 18,500 pg*hr/mL, greater than 19,000 pg*hr/mL, greater than 19,500 pg*hr/mL, or greater than 20,000 pg*hr/mL.

In certain embodiments, the one or more transdermal delivery patches or transdermal delivery patch system are maintained in contact with the skin surface of the subject in a manner sufficient to provide an area under the plasma dexmedetomidine concentration curve (AUC₅₋₉₆) from 15,000 pg*hr/mL to 20,000 pg*hr/mL, e.g., from 15,000 pg*hr/mL to 19,000 pg*hr/mL, from 15,000 pg*hr/mL to 18,000 pg*hr/mL, from 15,000 pg*hr/mL to 17,000 pg*hr/mL, from 15,000 pg*hr/mL to 16,000 pg*hr/mL, from 16,000 pg*hr/mL to 20,000 pg*hr/mL, from 16,000 pg*hr/mL to 19,000 pg*hr/mL, from 16,000 pg*hr/mL to 18,000 pg*hr/mL, from 16,000 pg*hr/mL to 17,000 pg*hr/mL, from 17,000 pg*hr/mL to 20,000 pg*hr/mL, from 17,000 pg*hr/mL to 19,000 pg*hr/mL, from 17,000 pg*hr/mL to 18,000 pg*hr/mL, from 18,000 pg*hr/mL to 20,000 pg*hr/mL, from 18,000 pg*hr/mL to 19,000 pg*hr/mL, or from 19,000 pg*hr/mL to 20,000 pg*hr/mL. In certain embodiments, the one or more transdermal delivery patches or transdermal delivery patch system are maintained in contact with the skin surface of the subject in a manner sufficient to provide a dexmedetomidine AUC₅₋₉₆ from 16,000 pg*hr/mL to 18,000 pg*hr/mL.

In certain embodiments, the one or more transdermal delivery patches or transdermal delivery patch system comprise from 2.5 mg to 3.5 mg dexmedetomidine (e.g., 2.92 mg dexmedetomidine) and are maintained in contact with the skin surface of the subject in a manner sufficient to provide a dexmedetomidine AUC₅₋₉₆ from 16,000 pg*hr/mL to 18,000 pg*hr/mL, e.g., 17,100 pg*hr/mL.

In certain embodiments, the one or more transdermal delivery patches or transdermal delivery patch system are maintained in contact with the skin surface of the subject in a manner sufficient to provide a maximal plasma concentration of dexmedetomidine (Cₘₐₓ) over 96 hours from 25 μg/mL to 400 μg/mL, e.g., from 25 μg/mL to 350 μg/mL, from 25 μg/mL to 300 μg/mL, from 25 μg/mL to 250 μg/mL, from 25 μg/mL to 200 μg/mL, from 25 μg/mL to 150 μg/mL, from 25 μg/mL to 100 μg/mL, from 25 μg/mL to 50 μg/mL, from 50 μg/mL to 400 μg/mL, from 50 μg/mL to 350 μg/mL, from 50 μg/mL to 300 μg/mL, from 50 μg/mL to 250 μg/mL, from 50 μg/mL to 200 μg/mL, from 50 μg/mL to 150 μg/mL, from 50 μg/mL to 100 μg/mL, from 100 μg/mL to 400 μg/mL, from 100 μg/mL to 350 μg/mL, from 100 μg/mL to 300 μg/mL, from 100 μg/mL to 250 μg/mL, from 100 μg/mL to 200 μg/mL, from 100 μg/mL to 150 μg/mL, from 200 μg/mL to 400 μg/mL, from 200 μg/mL to 350 μg/mL, from 200 μg/mL to 300 μg/mL, from 200 μg/mL to 250 μg/mL, from 250 μg/mL to 400 μg/mL, from 250 μg/mL to 350 μg/mL, from 250 μg/mL to 300 μg/mL, from 300 μg/mL to 400 μg/mL, or from 300 μg/mL to 350 μg/mL. In certain embodiments, the one or more transdermal delivery patches or transdermal delivery patch system are maintained in contact with the skin surface of the subject in a manner sufficient to provide a dexmedetomidine Cₘₐₓ over 96 hours from 250 μg/mL to 350 μg/mL or from 275 μg/mL to 325 μg/mL.

In certain embodiments, the one or more transdermal delivery patches or transdermal delivery patch system comprise from 2.5 mg to 3.5 mg dexmedetomidine (e.g., 2.92 mg) and are maintained in contact with the skin surface of the subject in a manner sufficient to provide a dexmedetomidine Cₘₐₓ over 96 hours from 25 μg/mL to 400 mg/mL, e.g., from 250 μg/mL to 350 μg/mL or from 275 μg/mL to 325 μg/mL.

In certain embodiments, the one or more transdermal delivery patches or transdermal delivery patch system are maintained in contact with the skin surface of the subject in a manner sufficient to provide a time for dexmedetomidine to reach the maximum concentration in the plasma or serum after administration (Tₘₐₓ) of greater than 10 hours, e.g., greater than 11 hours, greater than 12 hours, greater than 13 hours, greater than 14 hours, greater than 15 hours, greater than 16 hours, greater than 17 hours, greater than 18 hours, greater than 19 hours, or greater than 20 hours.

In certain embodiments, the one or more transdermal delivery patches or transdermal delivery patch system are maintained in contact with the skin surface of the subject in a manner sufficient to provide a dexmedetomidine Tₘₐₓ from 10 hours to 15 hours, e.g., from 10 hours to 14 hours, from 10 hours to 13 hours, from 10 hours to 12 hours, from 10 hours to 11 hours, from 11 hours to 15 hours, from 11 hours to 14 hours, from 11 hours to 13 hours, from 11 hours to 12 hours, from 12 hours to 15 hours, from 12 hours to 14 hours, from 12 hours to 13 hours, from 13 hours to 15 hours, from 13 hours to 14 hours, or from 14 hours to 15 hours. In certain embodiments, the one or more transdermal delivery patches or transdermal delivery patch system are maintained in contact with the skin surface of the subject in a manner sufficient to provide a dexmedetomidine Tₘₐₓ from 11 to 13 hours.

In certain embodiments, the one or more transdermal delivery patches or transdermal delivery patch system comprise from 2.5 mg to 3.5 mg dexmedetomidine (e.g., 2.92 mg) and are maintained in contact with the skin surface of the subject in a manner sufficient to provide a dexmedetomidine Tₘₐₓ of 10 hours to 15 hours, e.g., from 11 to 13 hours.

In certain embodiments, the one or more transdermal delivery patches or transdermal delivery patch system comprise from 2.5 mg to 3.5 mg dexmedetomidine (e.g., 2.92 mg) and are maintained in contact with the skin surface of the subject in a manner sufficient to provide two or more of the following: a dexmedetomidine AUC₅₋₉₆ from 15,000 pg*hr/mL to 20,000 pg*hr/mL, a dexmedetomidine Cₘₐₓ over 96 hours from 25 μg/mL to 400 μg/mL, and a dexmedetomidine Tₘₐₓ from 10 hours to 15 hours.

In certain embodiments, the one or more transdermal delivery patches or transdermal delivery patch system comprise from 2.5 mg to 3.5 mg dexmedetomidine (e.g., 2.92 mg) and are maintained in contact with the skin surface of the subject in a manner sufficient to provide two or more of the following: a dexmedetomidine AUC₅₋₉₆ from 16,000 pg*hr/mL to 18,000 pg*hr/mL, a dexmedetomidine Cₘₐₓ over 96 hours from 250 μg/mL to 350 μg/mL, and a dexmedetomidine Tₘₐₓ from 11 hours to 13 hours.

In certain embodiments, the one or more transdermal delivery devices or transdermal delivery patch system are maintained in contact with the skin surface of the subject in a manner sufficient to provide a non-sedative dosage of dexmedetomidine from 50 g/day to 350 g/day over the course of a dosage interval, e.g., from 100 g/day to 340 g/day, from 145 g/day to 330 g/day, from 155 g/day to 320 pg/day, from 165 g/day to 310 g/day, from 175 g/day to 300 g/day, from 185 g/day to 290 g/day, from 195 g/day to 280 g/day, and from 50 g/day to 250 g/day over the course of a dosage interval.

In certain embodiments, the target dosage is an amount that when applied to a subject provides for a systemic amount of dexmedetomidine that gives a desired mean plasma concentration of dexmedetomidine at specific times. In other embodiments, the target dosage is an amount that when applied to a subject provides for a steady state mean plasma concentration of the dexmedetomidine throughout a dosage interval or management protocol. In other embodiments, the target dosage is an amount that when applied to a subject provides for a particular rate of delivery of dexmedetomidine to the subject in vivo.

In certain embodiments, applying and maintaining one or more transdermal delivery patches or transdermal delivery patch system described herein delivers a target amount of dexmedetomidine, such as for example an average cumulative amount of dexmedetomidine delivered over the course of a dosage interval (e.g., 4 days or longer). The term “target cumulative amount” is meant the total quantity of dexmedetomidine that is delivered to the subject through the skin and may vary due to skin or mucous membrane permeability and metabolic activity of the site of application. In some embodiments, the average cumulative amount of dexmedetomidine may be 5 g/cm² or greater over a dosage interval (e.g., 3 days or longer), e.g., 25 g/cm² or greater, 50 g/cm² or greater, 75 g/cm² or greater, 100 g/cm² or greater, 125 g/cm² or greater, 200 g/cm² or greater, or 300 g/cm² or greater over the dosage interval. In certain embodiments, average cumulative amount of dexmedetomidine delivery over a dosage interval ranges such as from 5 g/cm² to 500 g/cm², such as from 25 g/cm² to 400 g/cm², or from 50 g/cm² to 350 g/cm². In some embodiments, the average cumulative amount of dexmedetomidine may be 200 g/cm² or greater over a dosage interval (e.g., 3 days or longer), e.g., 225 g/cm² or greater, 250 g/cm² or greater, 275 g/cm² or greater, 300 μg/cm² or greater, 325 μg/cm² or greater over the dosage interval. In certain embodiments, the average cumulative amount of dexmedetomidine delivery over a dosage interval is from 250 g/cm² to 350 g/cm², from 275 g/cm² to 350 g/cm², from 300 g/cm² to 350 g/cm², from 325 pg/cm² to 350 g/cm², from 250 g/cm² to 325 g/cm², from 275 g/cm² to 325 g/cm², from 300 g/cm² to 325 g/cm², from 250 g/cm² to 300 g/cm², from 275 g/cm² to 300 g/cm², or from 250 g/cm² to 300 g/cm².

The flux of an active agent by transdermal administration is the rate of penetration of the active agent through the skin or mucous membrane of the subject. In some instances, the flux of the dexmedetomidine can be determined by the equation:

$${J}_{skin flux} = P \times C$$

where J is the skin flux,
C is the concentration gradient across the skin or mucous membrane; and
P is the permeability coefficient.

Skin flux is the change in cumulative amount of drug that crosses a unit area (i.e., square cm) of the skin, other suitable skin source (e.g., skin obtained during abdominoplasty procedure), or mucous membrane with respect to time.

The transdermal dexmedetomidine flux may be determined using any convenient protocol, e.g., protocols employing human cadaver skin with epidermal layers (stratum corneum and viable epidermis) in a Franz cell having donor and receptor sides clamped together and receptor solution containing phosphate buffer. In one exemplary protocol, human cadaver skin is used, and epidermal layers (stratum corneum and viable epidermis) are separated from the full-thickness skin as skin membrane. Samples are die-cut with an arch punch to a final diameter of about 2.0 cm². The release liner of the transdermal delivery patch is removed, and the adhesive surface is adhered to the top of the epidermis/stratum corneum with the dexmedetomidine adhesive layer facing the outer surface of the stratum corneum. Gentle pressure is applied to effect good contact between the adhesive layer and stratum corneum. The donor and receptor sides of the Franz cell are clamped together and the receptor solution containing a phosphate buffer at pH 6.5 and 0.01% gentamicin is added to the Franz cell. The cells are kept at 32° C.-35° C. for the duration of the experiment. Samples of the receptor solution are taken at regular intervals and the active agent concentration is measured by HPLC. The removed receptor solution is replaced with fresh solution to maintain sink conditions. The flux is calculated from the slope of cumulative amount of the drug permeated into the receiver compartment versus time plot.

In certain embodiments, the transdermal delivery patch or transdermal delivery patch system provides an in vitro average flux of dexmedetomidine from 0.005 to 5 g/cm²/hr at any time after application of the patch and maintenance of contact between the skin surface and the patch (e.g., 24 hours, 48 hours, 72 hours, 96 hours and/or 120 hours after application), e.g., from 0.1 g/cm²/hr to 4 g/cm²/hr, from 0.1 g/cm²/hr to 3 g/cm²/hr, from 0.1 g/cm²/hr to 2 g/cm²/hr, or from 0.1 g/cm²/hr to 1 g/cm²/hr.

In certain embodiments, the transdermal delivery patch or transdermal delivery patch system provides an in vitro average flux of dexmedetomidine from 0.005 to 5 g/cm²/hr 24 hours after application, e.g., from 0.1 g/cm²/hr to 4 g/cm²/hr, from 0.1 g/cm²/hr to 3 g/cm²/hr, from 0.1 g/cm²/hr to 2 g/cm²/hr, or from 0.1 g/cm²/hr to 1 g/cm²/hr.

In certain embodiments, the transdermal delivery patch or transdermal delivery patch system provides an in vitro average flux of dexmedetomidine from 0.005 to 5 g/cm²/hr 48 hours after application, e.g., from 0.1 g/cm²/hr to 4 g/cm²/hr, from 0.1 g/cm²/hr to 3 g/cm²/hr, from 0.1 g/cm²/hr to 2 g/cm²/hr, or from 0.1 g/cm²/hr to 1 g/cm²/hr.

In certain embodiments, the transdermal delivery patch or transdermal delivery patch system provides an in vitro average flux of dexmedetomidine from 0.005 to 5 g/cm²/hr 72 hours after application, e.g., from 0.1 g/cm²/hr to 4 g/cm²/hr, from 0.1 g/cm²/hr to 3 g/cm²/hr, from 0.1 g/cm²/hr to 2 g/cm²/hr, or from 0.1 g/cm²/hr to 1 g/cm²/hr.

In certain embodiments, the transdermal delivery patch or transdermal delivery patch system provides an in vitro average flux of dexmedetomidine from 0.005 to 5 g/cm²/hr 96 hours after application, e.g., from 0.1 g/cm²/hr to 4 g/cm²/hr, from 0.1 g/cm²/hr to 3 g/cm²/hr, from 0.1 g/cm²/hr to 2 g/cm²/hr, or from 0.1 g/cm²/hr to 1 g/cm²/hr.

In certain embodiments, the transdermal delivery patch or transdermal delivery patch system provides an in vitro average flux of dexmedetomidine from 0.005 to 5 g/cm²/hr 120 hours after application, e.g., from 0.1 g/cm²/hr to 4 g/cm²/hr, from 0.1 g/cm²/hr to 3 g/cm²/hr, from 0.1 g/cm²/hr to 2 g/cm²/hr, or from 0.1 g/cm²/hr to 1 g/cm²/hr.

In certain embodiments, the transdermal delivery patch or transdermal delivery patch system provides an average flux of dexmedetomidine in vitro from 0.5 g/cm²/hr to 3 g/cm²/hr at any time after application of the patch and maintenance of contact between the skin surface and the patch (e.g., 24 hours, 48 hours, 72 hours, 96 hours and/or 120 hours after application), e.g., from 0.5 g/cm²/hr to 2 g/cm²/hr, or from 0.5 g/cm²/hr to 1 g/cm²/hr.

In certain embodiments, the transdermal delivery patch or transdermal delivery patch system provides an average flux of dexmedetomidine in vitro from 0.5 g/cm²/hr to 3 g/cm²/hr 24 hours after application, e.g., from 0.5 g/cm²/hr to 2 g/cm²/hr, or from 0.5 g/cm²/hr to 1 g/cm²/hr.

In certain embodiments, the transdermal delivery patch or transdermal delivery patch system provides an average flux of dexmedetomidine in vitro from 0.5 g/cm²/hr to 3 g/cm²/hr 48 hours after application, e.g., from 0.5 g/cm²/hr to 2 g/cm²/hr, or from 0.5 g/cm²/hr to 1 g/cm²/hr.

In certain embodiments, the transdermal delivery patch or transdermal delivery patch system provides an average flux of dexmedetomidine in vitro from 0.5 g/cm²/hr to 3 g/cm²/hr 72 hours after application, e.g., from 0.5 g/cm²/hr to 2 g/cm²/hr, or from 0.5 g/cm²/hr to 1 g/cm²/hr.

In certain embodiments, the transdermal delivery patch or transdermal delivery patch system provides an average flux of dexmedetomidine in vitro from 0.5 g/cm²/hr to 3 g/cm²/hr 96 hours after application, e.g., from 0.5 g/cm²/hr to 2 g/cm²/hr, or from 0.5 g/cm²/hr to 1 g/cm²/hr.

In certain embodiments, the transdermal delivery patch or transdermal delivery patch system provides an average flux of dexmedetomidine in vitro from 0.5 g/cm²/hr to 3 g/cm²/hr 120 hours after application, e.g., from 0.5 g/cm²/hr to 2 g/cm²/hr, or from 0.5 g/cm²/hr to 1 g/cm²/hr.

In certain instances, the transdermal delivery patch or transdermal delivery patch system provides an in vitro dexmedetomidine peak flux from 0.1 g/cm²/hr to 3 g/cm²/hr, e.g., from 0.1 g/cm²/hr to 2.5 g/cm²/hr, from 0.1 g/cm²/hr to 2.0 g/cm²/hr, from 0.1 g/cm²/hr to 1.5 g/cm²/hr, from 0.1 g/cm²/hr to 1 g/cm²/hr, or from 0.1 g/cm²/hr to 0.8 g/cm²/hr. In certain embodiments, the transdermal delivery patch or transdermal delivery patch system provides an in vitro dexmedetomidine peak flux from 0.5 to 3 g/cm²/hr, e.g., from 0.5 g/cm²/hr to 2.5 g/cm²/hr, from 0.5 g/cm²/hr to 2.0 g/cm²/hr, from 0.5 g/cm²/hr to 1.5 g/cm²/hr, or from 0.5 g/cm²/hr to 1 g/cm²/hr.

In some embodiments, peak dexmedetomidine flux is reached 2 hours or more after applying the transdermal delivery patch or transdermal delivery patch system, e.g., 4 hours or more, 6 hours or more, 12 hours or more, 18 hours or more, or 24 hours or more after applying the transdermal delivery patch or transdermal delivery patch system.

In certain embodiments, the methods herein further include administration of an analgesic rescue medication to the subject to control post-surgical pain following the surgical procedure. Typically, rescue medication is administered at the request of the subject (if human) or if the non-human subject demonstrates signs of pain.

In certain embodiments, the rescue medication can be administered from 0 to 96 hours after surgery, such as, for example, from 0 to 12 hours, from 0 to 24 hours, from 0 to 48 hours, from 0 to 72 hours, from 12 to 96 hours, from 24 to 96 hours, from 48 to 96 hours, from 72 to 96 hours, from 48 to 96 hours, from 48 to 72 hours, or from 72 hours to 96 hours.

In certain embodiments, the analgesic rescue medication is administered at least 6 hours after surgery, such as, for example, at least 8 hours after surgery, at least 10 hours after surgery, at least 12 hours after surgery, at least 24 hours after surgery, at least 48 hours after surgery, or at least 72 hours after surgery.

The analgesic rescue medication may be selected from an opioid, a non-opioid analgesic, and a combination thereof.

In certain embodiments, the analgesic rescue medication is an opioid. In certain embodiments, the opioid is selected from oxycodone, oxymorphone, hydrocodone, hydromorphone, fentanyl, morphine, codeine, methadone, tramadol, buprenorphine, and a combination thereof.

Use of the transdermal delivery patches described herein provides sustained post-operative analgesia in subjects such that reduced doses of rescue opioids are required compared to corresponding surgical procedures carried out without the one or more transdermal delivery patches. As such, in certain embodiments, applying the one or more transdermal delivery patches according to the methods described above reduces the amount of rescue opioid needed to treat post-operative pain by 1% or more by weight compared, on average, to a corresponding surgical procedure carried out without the one or more transdermal delivery patches, such as, for example, by 2% or more by weight, by 3% or more by weight, by 5% or more by weight, by 10% or more by weight, by 15% or more by weight, by 20% or more by weight, by 25% or more by weight, by 30% or more by weight, by 35% or more by weight, by 40% or more by weight, by 45% or more by weight, by 50% or more by weight, by 60% or more by weight, by 70% or more by weight, or by 80% or more by weight.

In certain embodiments, the rescue medication is a non-opioid analgesic. In certain embodiments, the non-opioid analgesic is selected from non-steroidal anti-inflammatory agents (NSAIDS), such as aspirin, ibuprofen, naproxen, celecoxib, acetaminophen, and combinations thereof. In certain embodiments, the non-opioid analgesic is ibuprofen.

In certain embodiments, the one or more transdermal delivery patches delivers a non-sedative amount of dexmedetomidine to the subject as measured by, e.g., the Wilson Sedation Score. In certain embodiments, the subject's Wilson Sedation Score does not exceed 3 post-operatively while the transdermal delivery patch is adhered to the skin surface of the subject, e.g., for at least 96 hours post-operatively. In certain embodiments, the subject's Wilson Sedation Score is less than 3, for example, 2 or 1. In certain embodiments, the subject's Wilson Sedation Score is 1. In certain embodiments, the one or more transdermal delivery patches or transdermal delivery patch system delivers a non-sedative amount of dexmedetomidine to the subject.

In certain embodiments, the non-sedative amount of dexmedetomidine is from 2.5 mg to 3.5 mg dexmedetomidine (e.g., 2.92 mg). In certain embodiments, the non-sedative amount of dexmedetomidine is less than 3.5 mg dexmedetomidine, for example, less than 3.4 mg dexmedetomidine, less than 3.3 mg dexmedetomidine, less than 3.2 mg dexmedetomidine, less than 3.1 mg dexmedetomidine, less than 3.0 mg dexmedetomidine, less than 2.9 mg dexmedetomidine, less than 2.8 mg dexmedetomidine, less than 2.7 mg dexmedetomidine, or less than 2.6 mg dexmedetomidine. In certain embodiments, the non-sedative amount of dexmedetomidine is less than 3.0 mg.

### 5. EXAMPLES

The following abbreviations used in the examples have the definitions set forth below:

| Abbreviation or | |
|---|---|
| Specialized Term | Explanation |
| AE | adverse event |
| ANCOVA | analysis of covariance |
| BMI | body mass index |
| BP | blood pressure |
| Cₘₐₓ | maximum observed plasma concentration |
| CRF | case report form |
| CV | coefficient of variation |
| DMTS | dexmedetomidine transdermal system |
| ECG | electrocardiogram |
| EOS | end-of-study |
| HEENT | head, ears, eyes, nose, and throat |
| HIV | human immunodeficiency virus |
| HR | heart rate |
| ICF | informed consent form |
| IV | intravenous |
| LOCF | last observation carried forward |
| MedDRA | Medical Dictionary for Regulatory Activities |
| NRS | numeric rating scale |
| NRSSPI | numeric rating scale summed pain intensity |
| PE | physical examination |
| PK | pharmacokinetic(s) |
| PT | preferred term |
| RR | respiratory rate |
| SAE | serious adverse event |
| SD | standard deviation |
| SE | standard error |
| SMC | safety monitoring committee |
| SOC | system organ class |
| SPI | summed pain intensity |
| SpO₂ | oxygen saturation |
| TEAE | treatment-emergent adverse event |
| WBC | white blood cell |
| WOCF | worst observation carried forward |

### Example 1: Preparation of an Exemplary Transdermal Delivery Patch

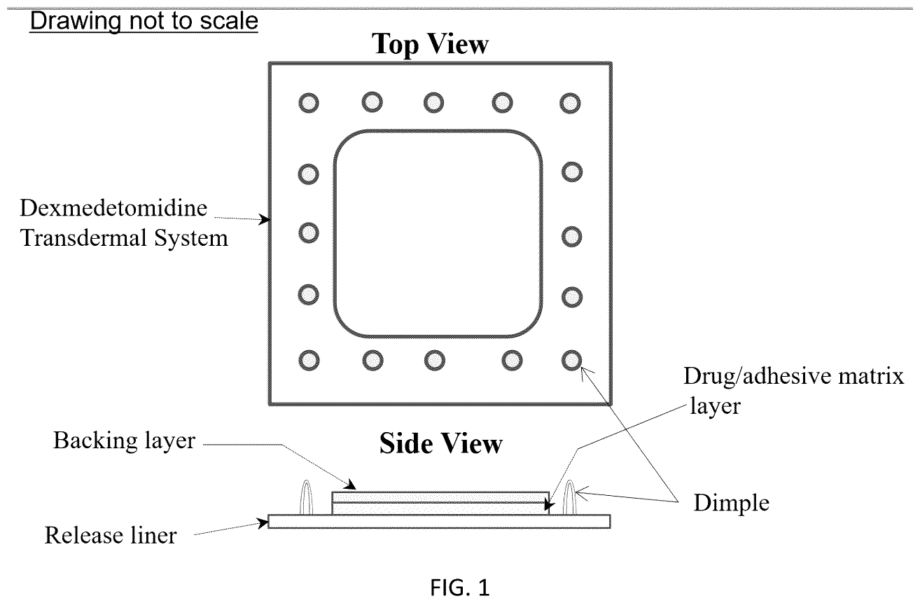

Formulations were prepared by mixing dexmedetomidine and a pressure sensitive adhesive in organic solvents (e.g., 30-60 wt % solid content in ethyl acetate, isopropyl alcohol, hexane, or heptane), followed by mixing. Once a homogeneous mixture was formed, the solution was cast on a release liner (siliconized polyester or fluoropolymer coated polyester sheets of 2-3 mils) and dried at 60°-80° C. for 10-90 minutes. The single layer adhesive films were then laminated to a backing (e.g., PET and/or polyethylene), cut to the desired size, and pouched. In some instances, lauryl lactate (LL) was added to the adhesive composition at the initial mixing step. A depiction of an exemplary DMTS (dexmedetomidine transdermal system) patch of the present disclosure is provided in FIG. 1.

### Example 2: Phase 2 Abdominoplasty Clinical Trial

### 1. Study Overview

A randomized, double-blind, placebo-controlled, one-time application study of DMTS or matching placebo over a 4-day treatment period (Day 1, Time 0 occurs when the DMTS or matching placebo systems have been applied) was completed with the following objectives:
Primary Objective: to evaluate the analgesic efficacy of DMTS, compared with placebo, in subjects following abdominoplasty.
Secondary Objectives: to assess the (1) safety and tolerability of the DMTS (including skin irritation), (2) the adhesion of the DMTS, and (3) the sedation effects of DMTS.

The primary outcome measure was the time-interval weighted summed pain intensity (SPI), measured using the 11-point (0 to 10) numeric rating scale (NRS), at designated time points from 5 to 96 hours following surgery.

The secondary outcome measures were:
The time-interval weighted SPI measured at rest over various time intervals.
The proportion of subjects using rescue analgesic medication.
Total dose of rescue analgesic medication.
Integrated assessments of pain score and rescue analgesic medication.

Other secondary outcome measures were:
The incidence and severity of treatment-emergent adverse events (TEAEs).
Clinically important changes in safety assessment results, including, as appropriate, vital signs, clinical laboratory tests, electrocardiograms (ECGs), and physical examinations.
Adhesion scores.
Sedation effect according to the Wilson sedation scale.

The exploratory measure was the time to first use of rescue analgesic medication.

Subjects scheduled for an abdominoplasty were screened up to 28 days prior to surgery. Eligible subjects were randomized equally to treatment with either DMTS or matching placebo (Table 1). Initially, subjects were enrolled and received DMTS/matching placebo corresponding to a dexmedetomidine dose of 3.65 mg (with a total surface area of 15 cm² provided by 2½ DMTS patches, each patch having a surface area of 6 cm² and containing 1.46 mg dexmedetomidine).

TABLE 1
Treatment Groups

| | | | |
|---|---|---|---|
| | Number of DMTS | Number of Placebo | Dexmedetomidine |
| Group | Applied | Systems Applied | Dose |

Target: 3.65 mg Dexmedetomidine (15 cm² surface area)

| | | | |
|---|---|---|---|
| Placebo | 0 | 2½ | 0 |
| DMTS | 2½ | 0 | 3.65 mg |

First dose reduction: 2.92 mg dexmedetomidine (12 cm² surface area)

| | | | |
|---|---|---|---|
| Placebo | 0 | 2 | 0 |
| DMTS | 2 | 0 | 2.92 mg |

Second dose reduction: 2.19 mg dexmedetomidine (9 cm² surface area)

| | | | |
|---|---|---|---|
| Placebo | 0 | 1½ | 0 |
| DMTS | 1½ | 0 | 2.19 mg |

The SMC periodically reviewed safety data (blinded or unblinded, if necessary) and could recommend a dose reduction based on the incidence of any observed clinically significant symptomatic bradycardia or clinically significant symptomatic hypotension. After 57 subjects had been dosed with 15 cm² DMTS, the SMC determined that based on bradycardia and hypotension, the dose was to be reduced to 2.92 mg dexmedetomidine (which was provided by 2 DMTS patches, each patch having a surface area of 6 cm² and containing 1.46 mg dexmedetomidine). Subsequent to this decision, enrolled subjects received DMTS/matching placebo corresponding to a dexmedetomidine dose of 2.92 mg.

Subjects resided at the clinical study unit for up to a total of 7 days. The surgical procedure, the intraoperative anesthesia, and the medication used for infiltration of the wound for local anesthesia before the last stitch were standardized.

During the postoperative period in the clinical study unit, recovery procedures were standardized (including rescue analgesic medication); and the following were performed: pain assessments (utilizing the NRS) at rest, sedation-level assessments, safety assessments (vital signs including oxygen saturation [SpO₂]), DMTS/matching placebo adhesion assessments, and skin irritation assessments. In addition, blood samples were collected for determination of plasma concentrations of dexmedetomidine.

### 2. Selection and Withdrawal of Subjects

Subjects were considered eligible to participate in this study if all the following inclusion criteria were satisfied:
Voluntarily provided written informed consent.
Male or female, ≥18 years of age.
Scheduled to undergo a full abdominoplasty (including repair of small incidental abdominal hernias but not including liposuction).
Had a physical status classification of 1 or 2 per the American Society of Anesthesiology.
Female subjects were eligible only if all the following applied:
   Not pregnant, not lactating, and not planning to become pregnant during the study or for 1 menstrual cycle thereafter; and
   Surgically sterile; or at least 2 years postmenopausal; or had a monogamous partner who is surgically sterile; or had a same gender sex partner; or is using double-barrier contraception; or practicing abstinence; or using an insertable, injectable, transdermal, or combination oral contraceptive for 3 months prior to the study, during the study, and for 1 month following the study.
Male subjects with female sex partners of childbearing potential must be surgically sterile or commit to use a reliable method of birth control during the study and for 1 month following the study.
Had a body weight>58 kg and a BMI of 20 to 38 kg/m², inclusive.
Able to understand the study procedures, comply with all study procedures, and agreed to participate in the study program for its full duration.

Subjects were not considered eligible to participate in this study if any one of the following exclusion criteria were met:
Known sensitivity to dexmedetomidine or any excipient in the DMTS/placebo or to any peri- or postoperative medication whose use was required in this study.
Skin abnormality (e.g., scar, tattoo) or unhealthy skin condition (e.g., burns, wounds) at the DMTS/matching placebo system application site, according to examination by the investigator at screening or admission to the clinic prior to surgery.
Clinically significant abnormal clinical laboratory test value as determined by the investigator.
History of deep vein thrombosis or factor V Leiden deficiency.
History of or positive test results for the human immunodeficiency virus (HIV), hepatitis B, or hepatitis C.
Clinically significant history or clinically significant manifestation of any of the following, as determined by the investigator: a renal, hepatic, cardiovascular, metabolic, neurologic, or psychiatric condition; congestive heart failure, peptic ulcer, gastrointestinal bleeding, or other condition that may preclude participation in the study.
History of physician-diagnosed migraine, frequent non-vascular headaches (>5 per month), seizures, or taking anticonvulsants.
Had another painful physical condition that may confound the assessments of postoperative pain, in the opinion of the investigator.
History of syncope or other syncopal attacks.
Present and/or significant history of postural hypotension (determined through examination by the investigator or designee), or history of severe dizziness or fainting on standing in the opinion of the investigator.
Evidence of a clinically significant 12-lead ECG abnormality.
Average heart rate (HR)<60 or >100 bpm, systolic blood pressure (BP)<90 or
   140 mmHg, or diastolic BP<60 or >90 mmHg, measured in 3 sequential positions (supine after 5 minutes; sitting after 2 minutes; and standing after 2 minutes) and after the sequence has been repeated 3 times.
History of alcohol abuse or prescription/illicit drug abuse within the previous 5 years.
Positive results on the urine drug screen or alcohol breath test indicative of drugs of abuse or alcohol use at screening and/or clinic check-in.
Received opioid therapy chronically for >2 weeks within the month prior to dosing of the study drug.
Received concurrent therapy that can interfere with the evaluation of efficacy or safety, such as any drug that in the investigator's opinion may exert significant analgesic properties or act synergistically with dexmedetomidine.
Used any natural health products (including chaparral, comfrey, germander, jin bu huan, kava, pennyroyal, skullcap, St. John's wort, or valerian, and excluding vitamins or mineral supplements) within 14 days prior to study drug administration and throughout the study, unless in the opinion of the investigator or designee, the product will not interfere with the study procedures or data integrity or compromise the safety of the subject.
Had symptoms of an upper respiratory tract infection within 14 days prior to dosing of the study drug.
Utilized oral or injectable corticosteroids within 14 days prior to dosing of the study drug (intranasal and topical corticosteroid use during this time period was allowed).
Received any investigational product within 30 days prior to dosing of the study drug.
Received DMTS in a previous clinical trial.
Hypersensitivity to opioids (including IV morphine), nonsteroidal anti-inflammatory drugs, or antibiotics.
In the opinion of the investigator or designee, was considered unsuitable for study entry and/or unlikely to comply with the study protocol for any reason.

In addition to the inclusion and exclusion criteria listed above, respectively, each subject agreed to abide by each of the following restrictions:
Subjects could not remove or tamper with the DMTS/matching placebo.
If the DMTS/matching placebo became <90% adhered, the subject alerted appropriate staff members at the clinical study unit.
Subjects avoided strenuous exercise, baths, saunas, steam-rooms or any other activities or environment that may lead to excessive sweating while the DMTS/matching placebo system was applied.
During treatment, subjects could take up to 1 shower daily provided the duration of the shower was limited to ≤10 minutes.
Subjects abstained from the following foods from 1 week prior to the first dose of study medication until the last PK sample was obtained: grapefruit juice or products, pomegranate juice or products, foods containing poppy seeds, and/or drinks or foods containing quinine (e.g., tonic water) or Seville oranges (e.g., orange marmalade).
Alcohol-containing beverages not permitted starting 2 days prior to surgery and while housed at the clinical study unit.

Subjects completed the study if they completed all screening procedures and the abdominoplasty, received study drug as intended, provided PK samples as described in the protocol, and completed all study and end of study (EOS) evaluations. Any subject with a treatment-emergent AE was followed until the AE resolved, returned to baseline, or deemed stable by the investigator. Subjects were advised that they were free to withdraw from the study at any time without consequence. Any subject who voluntarily withdrew from the study (e.g., withdrawal of consent) or is discontinued for a study-related reason (e.g., as a result of a treatment-emergent AE) prior to the completion of all assessments, was considered as having discontinued early from the study.

Subjects could discontinue the study or decide to withdraw themselves from the study under any of the circumstances listed below.
Death
Adverse event
A symptomatic clinically significant cardiovascular event (such as bradycardia or hypotension)
Protocol violation that warranted early discontinuation from the study as assessed by the investigator, designee, or sponsor
Lost to follow-up
Noncompliance
Pregnancy
Subject decision (withdrawal of consent when the reason was not known)
Termination of the study by the sponsor for any reason
Other: investigator or subject decision that cannot be classified to one of the categories above (in this case, the reason should be specified on the appropriate CRF)

### 3. Study Procedures

Subjects were randomized equally to treatment groups. The sponsor, the investigator, personnel at the clinical study unit directly involved with monitoring and/or performing study procedures and assessments, and the subjects were blinded to treatment assignment. The investigator could be unblinded to treatment in case of emergency if, and only if, knowledge of treatment assignment was needed to appropriately guide medical management.

Study drug (i.e., DMTS/matching placebo) was administered by qualified and trained clinical study unit personnel six to eight hours prior to the scheduled abdominoplasty. Following application, markings (such as lines) from the DMTS/matching placebo to the adjacent skin were made using indelible ink. Visual inspection of the integrity of these markings was performed to evaluate for any signs of tampering or removal of the DMTS or matching placebo and to assess the subject's compliance with appropriate wear.

The schedule of assessments and procedures is presented in Table 3.

TABLE 3
Schedule of events

| | Screening | Clinic | Treatment period | | | | | | End |
|---|---|---|---|---|---|---|---|---|---|
| | Days −28 | Check-In ^{a} | Day | Day | Day | Day | Day | Day | of |
| Assessments/Procedures | to −2 | Day −1 | 1 | 2 | 3 | 4 | 5 | 6 | study |
| Clinic Check-In | | CI | | | | | | CO | |
| (CI)/Check-Out (CO) | | | | | | | | | |
| Informed Consent | X | | | | | | | | |
| Medical History | X | X | | | | | | | |
| Inclusion/Exclusion | X | X | | | | | | | |
| Alcohol & Drug Screen | X | X | | | | | | | |
| Demographics | X | | | | | | | | |
| Vital Signs ^{b} | X | X | X | X | X | X | X | X | X |
| Physical Exam ^{c} | X | X | | | | | | | X |
| 12-Lead ECG | X | X | | | | | | | X |
| Clinical Safety | X | X ^{e} | | | | | | | X |
| Laboratory Tests ^{d} | | | | | | | | | |
| Serum Osmolality ^{f} | | | X | | | | | | |
| Serum Pregnancy Test | X | | | | | | | | |
| Urine Pregnancy Test | | X | | | | | | | X |
| HbsAg, HCV Ab, HIV | X | | | | | | | | |
| Randomization | | | X | | | | | | |
| Telemetry ^{g} | | X | X | X | X | X | X | | |
| DMTS/placebo applied | | | X | | | | | | |
| DMTS/placebo removed | | | | | | | X | | |
| Adhesion Assessment i | | | X | X | X | X | X | | |
| Skin Irritation | | | | | | | X | X | |
| Assessment j | | | | | | | | | |
| Blood PK collection | | | X | X | | | X | | |
| Sedation Assessment k | | | | X | X | X | X | X ^{l} | |
| Pain Assessment | | | X | X | X | X | X | | |
| Adverse effect | X | X | X | X | X | X | X | X | X |
| Concomitant Medications | X | X | X | X | X | X | X | X | X |

HbsAg = hepatitis B surface antigen;
HCV Ab = hepatitis C antibody
Days were defined as 24-hour intervals relative to the time of DMTS/matching placebo system application.
^{a} Clinic check-in occurred on the day prior to the scheduled surgery.
^{b} Obtained vital signs (BP, RR, HR, SpO₂) at screening, clinic check-in, during the treatment and washout periods and at EOS. Obtained oral or infrared body temperature at screening, clinic check-in, and EOS.
^{c} Performed a complete PE at screening and EOS and an abbreviated PE at clinic check-in. Measured height and body weight and calculated BMI at screening only.
^{d} Included hematology, serum chemistry, and urinalysis.
^{e} If screening laboratory tests were performed between Day −7 and Day −2, no additional testing was required prior to study entry. However, if screening laboratory tests were performed prior to Day −7, additional testing was to be performed within 7 days prior to check-in, and the results must have been available at check-in.
^{f} Blood for serum osmolality was collected prior to DMTS/matching placebo dosing and 3 hours after surgery.
^{g} Telemetry was used to monitor bradycardia; telemetry was started at least 12 hours before DMTS/matching placebo application and continued until 12 hours after DMTS/matching placebo removal.
h. After DMTS removal, the DMTS application site was swabbed for drug residue.
i Assessed DMTS/matching placebo adhesion at the following time points (±15 min): 6, 12, 24, 36, 48, 60, 72, and 84 hours after application and immediately prior to removal.
j Assessed skin irritation 1 hour (±5 min) and 24 hours (±2 h) after DMTS/matching placebo removal.
k Assessed sedation level once each day at approximately 12:00 pm (before lunch).
^{l} Subjects were not to be discharged from the clinic until or unless the sedation score was 1.

Vital signs consisted of BP, RR, HR, and SpO₂ and were measured (Table 3 and Table 4). Resting vital signs were obtained after the subject had been in a supine position (semi-supine following surgery) for at least 5 minutes. BP and HR were then obtained 2 minutes after the subject went from the supine (semi-supine) position to the sitting position and again 2 minutes after the subject stood from the sitting position. At screening, clinic check-in, and prior to dosing on Day 1, the sequence of supine, seated, and standing BP and HR was repeated for a total of 3 times. At all other time points, single measurements were obtained. Vital signs were also measured and recorded in the event of a symptomatic AE potentially associated with changes in vital signs. Any clinically significant abnormal vital sign was recorded on the AE CRF.

Following surgery, pulse oximetry was monitored continuously until subjects could tolerate oral medications. If oxygen saturation dropped below 90% on oral rescue analgesic medication, continuous monitoring was to be reinstated, as required.

Oral or infrared body temperature was measured at screening, clinic check-in, and EOS.

TABLE 4
Schedule of Vital Signs Assessments

| Day(s) | Assessment Time Points |
|---|---|
| Prior to the | Screening and Clinic check-in |
| Treatment Period | |
| 1-4 | Predose and 6, 12, 16, 24, 36, 48, 60, 72, and 84 |
| | hours after DMTS/matching placebo application |
| 5-6 | Before DMTS/matching placebo removal (i.e., 96 |
| Or at early | hours after application), 8, 16, 24, 32, and 40 hours |
| DMTS/matching | after removal |
| placebo removal | |

Clinically significant bradycardia in a given subject was defined as either an absolute HR<45 bpm or a drop of >30% relative to the mean HR value obtained prior to DMTS/matching placebo application and was sustained for 1 minute.

Clinically significant hypotension in a given subject was defined as the lower of either an absolute BP value (systolic BP<80 mmHg or diastolic BP<50 mmHg) or a drop of >30% relative to the respective mean BP value obtained prior to DMTS/matching placebo application, and this value (absolute value or drop relative to baseline) was sustained for 1 minute.

Continuous cardiac telemetry was used to monitor bradycardia, arrhythmias, and other cardiovascular events. Telemetry was started 12 hours before application of the DMTS/matching placebo and continued until 12 hours after DMTS/matching placebo removal. Any clinically significant abnormal finding that met the definition of an AE was to be reported as AEs and followed until resolution, returned to baseline, or was deemed clinically stable by the investigator.

A 12-lead ECG was to be obtained at screening, check-in, and EOS. ECGs were obtained after the subject had been resting for at least 5 minutes in the supine position. The ECG electronically measured and calculated ventricular rate and the PR, QRS, QT, and QTc intervals. The investigator was to review all ECG findings to assess whether any values outside the respective normal ranges were clinically significant. After the screening assessment, any clinically significant abnormal finding that met the definition of an AE was to be reported as an AE.

A PE, excluding breast, genitourinary, and rectal exam, was performed at screening and the EOS visit. The PE included assessment of the following body systems: head, ears, eyes, nose, and throat (HEENT), lymph nodes, heart, lungs, extremities, peripheral vascular, abdomen, neurological, chest/back, and skin. At screening only, the PE included height and weight measurements (and calculation of BMI). An abbreviated PE was performed at clinic check-in (Day −1) and included assessments of the following body systems: HEENT, heart, lungs, and skin. Any new or worsened physical finding (observed following administration of the DMTS/matching placebo) that met the definition of an AE was to be reported as an AE.

Adhesion of the DMTS/matching placebo was assessed visually at 6, 12, 24, 36, 48, 60, 72, and 84 hours after application and immediately prior to removal according to the schedule presented in Table 3. Adhesion was assessed according to the scoring described in Table 5.

TABLE 5
DMTS Adhesion Scale

| Score | Adherence Assessment |
|---|---|
| 0 | ≥95% of the DMTS/placebo system area adhered |
| 1 | ≥90% to <95% of the DMTS/placebo system area adhered |
| 2 | ≥85% to <90% of the DMTS/placebo system area adhered |
| 3 | ≥80% to <85% of the DMTS/placebo system area adhered |
| 4 | ≥75% to <80% of the DMTS/placebo system area adhered |
| 5 | ≥70% to <75% of the DMTS/placebo system area adhered |
| 6 | <70% of the DMTS/placebo system area adhered |
| 7 | DMTS/placebo system detachment |

Blood samples for determination of plasma concentrations of dexmedetomidine were obtained at the following time points: prior to dosing, and 3, 4, 6, 8, 10, 12, 24, and 96 hours after surgery.

### Screening (Days −28 to −2)

Subjects underwent screening procedures during the Screening period. Written informed consent was obtained before any protocol-specific procedures are performed. The following assessments and procedures were required during screening to determine eligibility:
Signed Informed Consent Form (ICF)
Drugs of abuse and alcohol testing
Medical history
Demographics
PE, including height and body weight measurements, and BMI calculation
Review of inclusion and exclusion criteria
Concomitant medication review
AE assessment
Vital signs
Oral or infrared temperature
Serology testing (hepatitis B surface antigen [HBsAg], hepatitis C antibody [HCV Ab], and HIV)
Serum pregnancy test (for female subjects of childbearing potential)
Clinical safety laboratory tests (hematology, serum chemistry, and urinalysis)
12-lead ECG
Obtain agreement to Other Restrictions

### Clinic Check-In (Day −1)

The assessments and procedures listed below were performed at the time of clinic check-in. Results of the assessments performed following check-in were reviewed and deemed acceptable by the investigator or designee before the subject was enrolled in the study.
Urine pregnancy test negative (female subjects of childbearing potential)
Drugs of abuse testing and alcohol testing
Review medical history
Confirm inclusion and exclusion criteria
Abbreviated PE
Concomitant medication review
AE assessment
Vital signs
Oral or infrared temperature
Review of clinical safety laboratory test results (hematology, serum chemistry, and urinalysis). Note: If screening laboratory testing was performed between Days −7 to −2, results were available at clinic check-in, and no additional laboratory testing prior to study entry. However, if screening laboratory testing occurred prior to Day −7, additional testing was performed within 7 days prior to check-in and the results of that testing were available at check-in.
12-lead ECG
Confirm agreement to Other Restrictions
Following confirmation of eligibility, subjects will be admitted to the clinic.
At least 12 hours before DMTS/matching placebo application, start cardiac telemetry monitoring; this will continue until 12 hours after DMTS/matching placebo removal (and may be interrupted, as needed, during transit to and from surgery as well as during surgery).
   Procedures within 12 Hours Prior to Surgery

Six to 8 hours prior to the scheduled abdominoplasty:
Collected a blood sample for PK analysis (predose sample)
Collected a blood sample for serum osmolality analysis
Reviewed telemetry for any events that would exclude the subject from participating in the study (e.g., clinically significant bradycardia, arrhythmia, or other cardiovascular event)
Applied the DMTS/matching placebo to a non-hairy site on the subject's upper arm.

Approximately 2 hours prior to the scheduled abdominoplasty:
Started IV hydration with lactated Ringer's solution at 500 mL/h and continued at this rate until the fasting fluid deficit was replaced (2 mL/kg per hour, nothing by mouth). Subsequently, continued IV hydration at approximately 2 mL/kg/h to replace insensible fluid loss throughout the perioperative period until the subject was able to take adequate fluid by mouth and was hemodynamically stable.

Subjects were administered general anesthesia according to a standard regimen (Table 6).

TABLE 6
General Anesthesia Regimen

| | |
|---|---|
| Monitors | All routine required American Society of Anesthesiologists monitors |
| IV Fluids | As described above |
| Premedication | Midazolam 1-3 mg IVP as needed |
| Induction | Propofol 1-3 mg/kg IV |
| | Rocuronium 0.6-1.2 mg/kg IV |
| | Fentanyl 1-2 μg/kg IV |
| Maintenance | No N₂O |
| | Air/O₂ as required to maintain intraoperative O₂ saturation >95% |
| | Sevoflurane or desflurane are preferred agents at 0.8 to 1.2 minimum |
| | alveolar concentration (MAC) as tolerated |
| | Isoflurane at 0.8 to 1.2 MAC as tolerated can be used only if both |
| | sevoflurane and desflurane are not available |
| | Rocuronium 0.1 to 0.2 mg/kg as needed per train-of-four (TOF) monitoring |
| | and at the discretion of the anesthesia provider |
| | Periodic propofol boluses up to 0.5 mg/kg IV to optimize depth of anesthesia |
| | and reduce intraoperative fentanyl use |
| | Fentanyl up to 4 μg/kg IV as needed for a total maximum perioperative |
| | fentanyl dose of 6 μg/kg |
| Airway device | Oral endotracheal tube or supraglottic device (laryngeal mask airway, etc.) at |
| | the discretion of the anesthesia provider |
| Reversal of | The selection of the reversal agent(s) is at the discretion of the anesthesia |
| neuromuscular blockade | provider. |
| (NMB) if needed | |
| Local anesthetic | 0.2% ropivacaine up to the maximum per kg dose with epinephrine |
| administered by surgeon | |

IV = intravenous; IVP = intravenous push; MAC = minimum alveolar concentration; NMB = neuromuscular blockade; TOF = train-of-four

Pulse oximetry was monitored continuously following surgery until subjects could tolerate oral medications. If O₂ saturation dropped below 90% on oral rescue analgesic medication continuous monitoring was reinstated.

### Treatment Procedures

During the treatment period, subjects received a single 4-day (96-hour) application of DMTS or matching placebo. The treatment period started (Day 1 Time 0) when the DMTS or matching placebo systems were applied. As needed, subjects received rescue analgesic medications.

### Study Drug Materials and Administration

The DMTS provided extended release of dexmedetomidine over a 4-day application period. Each transdermal delivery patch had a drug layer affixed to a backing layer. The drug layer contained 1.46 mg of dexmedetomidine, a pressure sensitive adhesive comprising a hydroxyl functionalized acrylate polymer, and lauryl lactate. The drug layer was adhered to a siliconized polyester or fluoropolymer coated polyester, wherein the release liner, when removed, exposed an adhesive surface of the drug layer suitable for adhesion to a skin surface; the backing layer comprised polyethylene and polyethylene terephthalate; and the transdermal delivery patch was unitary in structure. In some instances, crosslinked polyvinylpyrrolidone (PVP-CLM), polyvinylpyrrolidone K90 (PVP K90), levulinic acid (LA), oleic acid (OA), lauryl lactate (LL), or propylene glycolmonolaurate (PGML) was added to the adhesive composition.

Each DMTS had a surface area of 6 cm² and contained 1.46 mg of dexmedetomidine. Excipients included lauryl lactate and an acrylate-based copolymer. The matching placebo transdermal system was identical to the DMTS, except that it did not contain dexmedetomidine.

Subjects received 2 or 2½ DMTS patches (each DMTS had a surface area of 6 cm² and 1.46 mg dexmedetomidine) or matching placebo 6 to 8 hours prior to the scheduled abdominoplasty; each DMTS/matching placebo was applied to a non-hairy portion of the subject's upper arm by trained clinical study unit personnel. The ½ DMTS/matching placebo (corresponding to 3 cm²) was cut from a whole DMTS/matching placebo. The DMTS/matching placebo was worn for 4 days (96 hours from the time of application).

### 4. Assessment of Efficacy and Pharmacodynamics

Pain intensity was assessed at 1, 2, 3, 4, 5, 6, 7, 8, 10, 12, 16, 24, 32, 40, 48, 56, 64, 72, 84, and 96 hours after surgery and prior to administration of any rescue medication. Subjects evaluated their pain intensity using an 11-point (0-10) Numeric Rating Scale (NRS) at rest.

The NRSSPI over 5 to 96 hours after surgery were analyzed using an analysis of covariance (ANCOVA) model that included treatment (DMTS or placebo) as an independent variable and clinical study unit as a covariate.

NRSSPI measured at rest over other time intervals (i.e., 5 to 6, 7, 8, 10, 12, 16, 24, 32, 40, 48, 56, 64, 72, 84, and 96 hours after surgery) were calculated and compared between subjects randomized to placebo and those randomized to DMTS using the same ANCOVA model as used for the primary endpoint.

The proportions of subjects using rescue pain medication over 0 to 5, 6, 7, 8, 10, 12, 16, 24, 32, 40, 48, 56, 64, 72, 84, and 96 hours after surgery were summarized using descriptive statistics.

The difference in percent of subjects using rescue pain medication between the DMTS and placebo groups were evaluated using Barnard's unconditional exact test for the proportion difference.

The time to first use of rescue pain medication (morphine, ibuprofen, or oxycodone) was calculated as the duration from the end of surgery to the first use of rescue pain medication and was summarized using descriptive statistics and Kaplan-Meier curves. The difference in time to first use of rescue pain medication between the DMTS and placebo groups were analyzed using a Cox proportional hazards model with clinical study unit as a covariate.

The use of rescue pain medication over 0 to 5, 6, 7, 8, 10, 12, 16, 24, 32, 40, 48, 56, 64, 72, 84, and 96 hours after surgery were summarized as frequency of use and dose taken of morphine, ibuprofen, and oxycodone. The dose of each analgesic medication taken was analyzed using ANCOVA models where the response variable was the dose of each rescue analgesic medication over each time interval. The models included treatment (DMTS or placebo) as an independent variable and clinical study unit as a covariate.

An integrated variable of pain scores and rescue medication was calculated using the method described by Silverman (see Silverman DG, O'Connor T Z, Brull S J. Integrated assessment of pain scores and rescue morphine use during studies of analgesic efficacy. Anesth Analg. 1993; 77:168-70). The integrated assessments over 5 to 6, 7, 8, 10, 12, 16, 24, 32, 40, 48, 56, 64, 72, 84, and 96 hours after surgery were summarized using descriptive statistics and analyzed using the Wilcoxon rank-sum test.

All medications taken by the subjects from screening through completion of the end-of-study (EOS) visit were documented.

All subjects received IV acetaminophen (1000 mg every 6 hours±30 minutes) starting 60 minutes after surgery (last stitch) through 91 hours after surgery.

Subjects with inadequately controlled pain symptoms could request rescue analgesia. The use of rescue analgesic medications was monitored throughout the study. If a subject requested rescue medication within 30 minutes prior to the next scheduled dose of acetaminophen, then the next dose of acetaminophen was administered. If a subject requested rescue medication during the 6-hour acetaminophen lockout period, then oral ibuprofen, 400 mg every 4 hours (lockout period), was administered. If a subject requested rescue medication during the 4-hour ibuprofen lockout period and during the acetaminophen lockout period, then oxycodone, 5 mg every 4 hours, was administered. If a subject requested additional rescue medication for breakthrough pain during the first hour after surgery, the 2 mg IV morphine every 15 minutes was administered as needed. For the next 11 hours, if the subject requested additional rescue medication and was ‘locked out’ of the other rescue analgesics (as noted above), then 2 mg IV morphine every hour was administered. Sedation level was assessed once each day, starting on Day 2, between 11:00 am and 12:00 pm (but before lunch) and at the time of discharge. Sedation level was evaluated using the criteria defined in the original Wilson sedation scale presented in Table 7. Sedation scores over time were summarized. Sedation data collected after early discontinuation of study treatment were excluded from the data summary.

TABLE 7
Original Wilson Sedation Scale

| Score | Description |
|---|---|
| 1 | Fully awake and oriented |
| 2 | Drowsy |
| 3 | Eyes closed but rousable to command |
| 4 | Eyes closed but rousable to mild physical stimulation |
| | (earlobe tug) |
| 5 | Eyes closed and unrousable to mild physical stimulation |

### 5. Safety Assessment

An AE was any untoward medical occurrence associated with the use of a drug in humans, whether or not considered related to the drug. An AE was any unfavorable or unintended sign (including a clinically significant abnormal laboratory finding), symptom, or disease temporally associated with the use of a drug or investigational product, without any judgment about causality. An AE could have occurred from any use of the drug and from any route of administration, formulation, or dose, including an overdose.

Adverse events (AEs) were ascertained on the basis of volunteered signs or symptoms and by clinical observation and assessment during study visits. All severe AEs (SAEs) and unresolved AEs were followed with appropriate medical management until resolution, return to baseline, or a stable status was achieved. Pre-existing conditions, diseases, or disorders were not considered treatment-emergent AEs unless there was a change in intensity, frequency, or quality following study drug administration. AEs were classified in accordance with Table 8.

TABLE 8
Classification of Adverse Event Severity

| Classification | Definition |
|---|---|
| Mild | An event that is usually transient and requires only minimal treatment or |
| | therapeutic intervention. The event does not generally interfere with usual |
| | activities of daily living. |
| Moderate | An event that is alleviated with additional specific therapeutic intervention. The |
| | event may interfere with usual activities of daily living or cause discomfort but |
| | poses no significant or permanent risk of harm to the subject. |
| Severe | An event that requires intensive therapeutic intervention. The event interrupts |
| | usual activities of daily living or significantly affects clinical status. The event |
| | may pose a significant risk of harm to the subject and hospitalization may be |
| | required. |

AEs were to be collected from the time the ICF was signed until the EOS visit. Pre-existing conditions, diseases, or disorders were not considered TEAEs unless there was a change in intensity, frequency, or quality following study drug administration.

AEs were to be followed until resolution, return to baseline, or a stable status had been achieved as determined by the investigator or designee. Any subject with an ongoing AE who did not return for a follow-up visit was contacted 3 times by the site to arrange for a follow-up visit. The third and final attempt to contact the subject was to be completed by sending a certified letter.

All attempts of contact were to be documented in the subject's source documents. If no response was received from the subject after 3 attempts, the subject was to be removed from the study and considered lost to follow-up.

An SAE was defined as an AE that resulted in any of the following outcomes:
Death
A life-threatening AE (i.e., the AE placed the subject at immediate risk of death; an AE that hypothetically might have caused death if it were more severe was not an SAE by this criterion)
An in-patient hospitalization or prolongation of an existing hospitalization
A persistent or significant incapacity or substantial disruption of the ability to conduct normal life functions
A congenital anomaly/birth defect
An important medical event that may not have resulted in death, may not have been life-threatening, or may not have required hospitalization, but based on appropriate medical judgment, may have jeopardized the subject or may have required medical or surgical intervention to prevent the outcomes listed above.

In the event of pregnancy in a female subject, the clinical staff was to report the pregnancy to the sponsor within 24 hours of learning of the event. Any subject who became pregnant during the study was to be immediately discontinued from study treatment.

All AE verbatim terms were coded using the Medical Dictionary for Regulatory Activities (MedDRA; version 25.0) and summarized by system organ class (SOC), preferred term (PT), and by treatment group. An AE or SAE was regarded as treatment-emergent if it started or worsened (e.g., increased in severity or frequency) during or after the application of study treatment. TEAEs were summarized by severity, relationship to treatment, seriousness, and those leading to discontinuation of study medication. In addition, application site reactions designated as TEAEs were summarized.

Skin irritation at each DMTS/matching placebo site was assessed 1 hour (±5 min) and 24 hours (±2 h) after removal of the DMTS/matching placebo. Skin irritation assessment(s) included an evaluation of the presence and severity of erythema, edema, papules, and vesicles (if any) using the scale in Table 9.

TABLE 9
Skin Irritation Scoring Scale

| Score | Erythema | Edema | Papules | Vesicles |
|---|---|---|---|---|
| 0 | None | None | None | None |
| 1 | Minimal/Mild | Minimal/Mild | Minimal/Mild | Minimal/Mild |
| | (diffuse, barely | (barely visible; skin | (<10 seen per | (<10 seen per |
| | visible, locally | elevated <1 mm) | application area; | application area; |
| | restricted) | | non-confluent) | nonconfluent) |
| 2 | Moderate | Moderate | Moderate | Moderate |
| | (definite redness) | (skin elevated | (≥10 observed on | (≥10 observed on |
| | | ~1 mm) | <50% of application | <50% of application |
| | | | area; ±increasing | area; ±increasing |
| | | | confluence) | confluence) |
| 3 | Severe | Severe | Severe | Severe |
| | (strong fiery redness; | (skin raised >1 mm) | (≥10 observed on | (≥10 observed on |
| | may spread beyond | | ≥50% of application | ≥50% of application |
| | local application | | area; ±increasing | area; ±increasing |
| | area) | | confluence) | confluence) |

Whenever a skin irritation assessment resulted in a non-zero score, the symptom was documented and tracked as an AE. Any subject with a skin irritation score of 1 was followed through resolution or stabilization. Any skin irritation or other local effect at the study drug application site that was not included in the skin irritation scale and/or identified outside of the scheduled skin irritation assessment time points was recorded as an AE and designated as an application site reaction.

Erythema, edema, papule, and vesicle scores and total irritation scores (calculated as the sum of skin irritation scores) were summarized using descriptive statistics. All skin irritation scores were summarized.

Clinical laboratory tests were summarized by descriptive statistics or frequency distribution as appropriate. Clinical laboratory tests were also summarized by shift tables. Abnormal findings (i.e., values outside the normal range) were included.

Vital signs measurements and changes at each visit from baseline were summarized and listed. ECG parameters and changes at each visit from baseline were summarized; subjects with clinically relevant abnormal findings or significant changes during the study were summarized and listed.

PE results that were deemed by the investigator as clinically significant abnormalities were tabulated by type of exam, study visit, and treatment group; clinically significant abnormalities were also listed. Concomitant medications reported during the study were listed by subject.

For each AE, the investigator or designee assessed causality (i.e., relationship between the AE and study drug), according to the definitions provided in Table 10.

TABLE 10
Classifications for Adverse Event Causality (Relationship to Study Drug)

| Classification | Definition |
|---|---|
| Not Related | The adverse event was clearly not related to study drug, with no reasonable |
| | relationship between the experience and the administration of study drug, but |
| | rather, related to other etiologies such as concomitant medications or subject's |
| | clinical state. Evidence for this classification must have been documented and |
| | specified on the case report form (CRF) by the investigator (e.g., no temporal |
| | relationship, and/or inconsistent with pharmacology, and/or specific alternative |
| | cause, etc.). (As a guideline, events that were <5% likely to be drug related |
| | might have been characterized as not related.) |
| Unlikely Related | Relationship to study drug was doubtful. The adverse reaction followed a |
| | plausible temporal sequence from administration of the study drug, but |
| | otherwise was not consistent with a known or likely pharmacological or |
| | clinical response pattern to the suspected study drug, and moreover, the |
| | reaction was more likely to have been produced by the subject's clinical state |
| | or other modes of therapy administered to the subject. (As a guideline, events |
| | that were 5% to 15% likely to be drug related might have been characterized as |
| | unlikely related.) |
| Possibly Related | Relationship to study drug was plausible. The adverse reaction followed a |
| | plausible temporal sequence from administration of the study drug and also |
| | followed a known or likely pharmacological or clinical response pattern to the |
| | suspected study drug. Although the reaction might have been produced by the |
| | subject's clinical state or other modes of therapy administered to the subject, |
| | the influence of study drug must have been considered. (As a guideline, events |
| | that were 16% to 90% likely to be drug related might have been characterized |
| | as possibly related.) |
| Related | Relationship to study drug was clearly, definitely, or highly likely, with no |
| | other plausible explanation. The adverse reaction followed a plausible |
| | temporal sequence from administration of the study drug and followed a |
| | known pharmacological or clinical response pattern to the suspected study |
| | drug. Moreover, the reaction could not be reasonably explained by the known |
| | characteristics of the subject's clinical state or other modes of therapy |
| | administered to the subject. (As a guideline, events that were >90% likely to |
| | be drug related might have been characterized as related.) |

### 6. Assessment of Pharmacokinetics

Venous blood samples for PK analyses were obtained at the following time points: prior to dosing, and 3, 4, 6, 8, 10, 12, 24, and 96 hours after surgery. Plasma concentrations of dexmedetomidine were determined by KCAS, LLC (Olathe, KS) using a validated analytical method.

Plasma concentrations of dexmedetomidine at each time point were summarized using descriptive statistics. Any plasma concentration data collected after early discontinuation were excluded from the data summary but were included in a listing.

The PK parameters of AUC_{0-inf}, AUC₅₋₉₆, Cₘₐₓ, and Tₘₐₓ were summarized using descriptive statistics. In addition, AUC₅₋₉₆ between the following subgroups were compared using a two-sample t-test: sex (male vs female), race (white vs non-white), age (≤median vs >median), body weight (≤median vs >median) and the patch size (15 cm² vs 12 cm²).

For AUC₅₋₉₆, blood sampling that occurred before hour 96 and within the analysis window of hour 96 (e.g., sample taken at hour 95.8), the nominal time point (i.e., hour 96) was used as the collection time for calculating AUC₅₋₉₆. Cₘₐₓ was derived using the following rules:
Predose values were not included in the determinations.
Analyses were based on actual sampling times.
Any concentration below the lower limit of quantitation was reported as 0.00 for summaries by each time point.

### 7. Results

A total of 179 subjects were enrolled in the study and 167 were randomized to treatment as follows: 85 to placebo and 82 to DMTS (57 received DMTS 15 cm² and 25 received DMTS 12 cm²). A total of 136 subjects completed the study, and 31 (17 in the placebo group and 14 across the DMTS groups) discontinued the study early. Of the 31 subjects who discontinued early, 18 (8 placebo, 10 DMTS) completed treatment but did not complete the follow-up visit and 13 (9 placebo, 4 DMTS) discontinued treatment early.

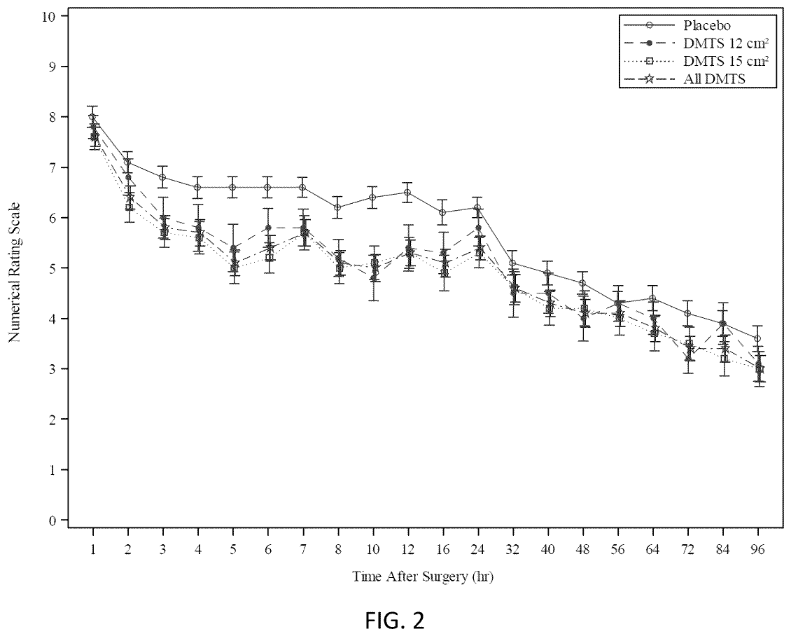

Pain intensity (Numeric Rating Scale, “NRS”) after surgery was compared between DMTS and placebo groups using an analysis of covariance (ANCOVA) model. The NRS had a scale of 0 (no pain) to 10 (worst possible pain). The results are shown in FIG. 2. The NRS score decreased over time after surgery as expected for all three groups. The NRS for the placebo group remained the highest compared to the DMTS groups.

### 7.1 Demographic and Other Baseline Characteristics

Demographic and baseline characteristics are summarized for the Safety Analysis Population by treatment group and overall in Table 11. Overall, subjects had a mean age of 40.1 years (with a range of 22 to 67 years); most subjects were female (98.2%), most were white (94.6%), and most were not Hispanic or Latino (69.5%). At baseline, subjects had a mean BMI of 29.8 kg/m2 (with a range of 21 to 38 kg/m2).

TABLE 11
Demographic and Baseline Characteristics (Safety Analysis Population)

| | | DMTS | | | |
|---|---|---|---|---|---|
| | All Placebo | 12 cm² | 15 cm² | All DMTS | Overall |
| Characteristic | N = 85 | N = 25 | N = 57 | N = 82 | N = 167 |
| Age, years | | | | | |
| Mean (SD) | 40.0 (8.96) | 41.1 (10.09) | 39.9 (10.32) | 40.2 (10.20) | 40.1 (9.56) |
| Median | 40.0 (22, 65) | 41.0 (25, 67) | 38.0 (22, 62) | 38.0 (22, 67) | 39.0 (22, 67) |
| Range (min, max) | | | | | |
| Gender, n (%) | | | | | |
| Male | 2 (2.4) | 0 (0.0) | 1 (1.8) | 1 (1.2) | 3 (1.8) |
| Female | 83 (97.6) | 25 (100. 0) | 56 (98.2) | 81 (98.8) | 164 (98.2) |
| Race, n (%) | | | | | |
| Asian | 1 (1.2) | 0 (0.0) | 0 (0.0) | 0 (0.0) | 1 (1.06) |
| Black | 0 (0.0) | 0 (0.0) | 1 (1.8) | 1 (1.2) | 1 (0.6) |
| Native Hawaiian or | 1 (1.2) | 1 (4.0) | 0 (0.0) | 1 (1.2) | 2 (1.2) |
| Other Pacific Islander | | | | | |
| White | 82 (96.5) | 24 (96.0) | 52 (91.2) | 76 (92.7) | 158 (94.6) |
| Other | 1 (1.2) | 0 (0.0) | 4 (7.0) | 4 (4.9) | 5 (3.0) |
| Ethnicity, n (%) | | | | | |
| Hispanic or Latino | 21 (24.7) | 10 (40.0) | 18 (31.6) | 28 (34.1) | 49 (29.3) |
| Not Hispanic or | 63 (74.1) | 15 (60.0) | 38 (66.7) | 53 (64.6) | 116 (69.5) |
| Latino | | | | | |
| Not Reported | 1 (1.2) | 0 (0.0) | 1 (1.8) | 1 (1.2) | 2 (2.1) |
| BMI, kg/m2 | | | | | |
| Mean (SD) | 29.7 (3.97) | 30.7 (4.53) | 29.4 (4.31) | 29.8 (4.39) | 29.8 (4.17) |
| Median | 29.3 (22, 38) | 31.5 (24, 38) | 29.0 (21, 38) | 29.7 (21, 38) | 29.3 (21, 38) |
| Range (min, max) | | | | | |

For the 82 subjects randomized to DMTS, the overall mean (SD) duration of exposure was 94.2 (9.90) hours, with a median of 96.0 hours and a range of 58 to 97 hours.

For most subjects, DMTS adhesion scores were 0 (indicating that >95% of the DMTS/matching placebo area was adhered) throughout the duration of application.

### 7.2 Efficacy

The primary hypothesis was that DMTS was superior (i.e., greater analgesic efficacy) to placebo as measured by the numeric rating scale summed pain intensity (NRSSPI) over 5 to 96 hours after surgery. The significance level was set at 0.05. The statistical null hypothesis was that the NRSSPI over 5 to 96 hours after surgery of the DMTS group was equal to that of the placebo group.

Subjects who received DMTS had statistically significantly less post-surgical pain over 5 to 96 hours after surgery, as measured by the NRSSPI. The LS mean NRSSPI measured at rest over 5 to 96 hours after surgery was 500.9 for the placebo group and 442.4 for the All DMTS group. The LS mean treatment difference was −58.5 (95% CI: −111, −6; p=0.0280) (Table 12).

TABLE 12
NRSSPI Measured at Rest Over 5 to 96 Hours After Surgery (ITT Analysis Population)

| | | | |
|---|---|---|---|
| All Placebo | DMTS 12 cm² | DMTS 15 cm² | All DMTS |
| N = 85 | N = 25 | N = 57 | N = 82 |

5 to 96 Hours After Surgery

| | | | | |
|---|---|---|---|---|
| n | 84 | 25 | 57 | 82 |
| Mean (SD) | 441.2 (155.95) | 394.1 (132.52) | 375.7 (192.86) | 381.3 (176.04) |
| Median | 427.8 | 367.0 | 359.0 | 360.0 |
| Range (min, max) | (91, 812) | (221, 637) | (74, 900) | (74, 900) |
| LS Mean (SE) | 500.9 (85.48) | 455.2 (91.99) | 436.8 (88.40) | 442.4 (87.52) |

Treatment Difference (DMTS−Placebo)

| | | | |
|---|---|---|---|
| LS Mean | −45.7 (38.77) | −64.1 (29.23) | −58.5 (26.46) |
| Difference (SE) | | | |
| 95% CI of LS | (−122, 31) | (−122, −7) | (−111, −6) |
| Mean Difference | | | |
| p-value | 0.2398 | 0.0293 | 0.0280 |

(1) The numeric rating scale (NRS) has a range of 0 (no pain) to 10 (worst possible pain); a negative treatment difference (DMTS−Placebo) favors DMTS.
(2) Inferential statistics were from an ANCOVA model with treatment as independent variable, and clinical study unit as a covariate.

Evaluation of the NRSSPI over the time intervals of 5 to 6, 7, 8, 10, 12, 16, 24, 32, 40, 48, 56, 64, 72, 84 and 96 hours after surgery showed that subjects in the All DMTS group had statistically significantly lower NRSSPI scores at each time interval than the placebo group (Table 13).

Sensitivity analyses of the NRSSPI measured at rest over time using LOCF or WOCF imputation for pain assessments collected during the efficacious windows of the rescue medications produced similar results, with subjects in the All DMTS group having statistically significantly lower NRSSPI scores at each time interval than the placebo group.

TABLE 13
NRSSPI from 5 hours through 96 hours after surgery in DMTS and placebo groups

| | LS Mean (SE) | | LS Mean | |
|---|---|---|---|---|
| | All Placebo | All DMTS | Difference | |
| Timing | N = 85 | N = 82 | (95% CI) | p-value |
| 5 to 6 hours after surgery | 7.5 (1.03) | 6.2 (1.05) | −1.3 (−2, −1) | <0.0001 |
| 5 to 7 hours after surgery | 15.3 (1.99) | 13.0 (2.04) | −2.4 (−4, −1) | 0.0002 |
| 5 to 8 hours after surgery | 22.3 (2.87) | 18.9 (2.94) | −3.3 (−5, −2) | 0.0002 |
| 5 to 10 hours after surgery | 35.1 (4.57) | 29.2 (4.68) | −5.8 (−9, −3) | 0.0002 |
| 5 to 12 hours after surgery | 48.5 (6.29) | 40.1 (6.44) | −8.4 (−12, −5) | <0.0001 |
| 5 to 16 hours after surgery | 76.1 (9.96) | 62.7 (10.30) | −13.4 (−20, −7) | 0.0001 |
| 5 to 24 hours after surgery | 132.6 (17.33) | 112.8 (17.75) | −19.8 (−30, −9) | 0.0003 |
| 5 to 32 hours after surgery | 185.2 (24.47) | 160.2 (25.05) | −25.1 (−40, −10) | 0.0011 |
| 5 to 40 hours after surgery | 235.3 (32.22) | 205.7 (32.99) | −29.5 (−49, −10) | 0.0034 |
| 5 to 48 hours after surgery | 282.3 (39.81) | 248.1 (48.47) | −34.3 (−59, −10) | 0.0058 |
| 5 to 56 hours after surgery | 324.4 (47.34) | 286.8 (48.47) | −37.6 (−66, −9) | 0.0110 |
| 5 to 64 hours after surgery | 367.8 (55.08) | 327.0 (56.59) | −40.9 (−74, −7) | 0.0173 |
| 5 to 72 hours after surgery | 406.8 (62.75) | 360.7 (64.25) | −46.2 (−87, −8) | 0.0183 |
| 5 to 84 hours after surgery | 454.8 (74.05) | 401.7 (75.82) | −53.1 (−98, −8) | 0.0213 |
| 5 to 96 hours after surgery | 500.9 (85.48) | 442.4 (87.52) | −58.5 (−122, −7) | 0.0280 |

(1) The numeric rating scale (NRS) has a range of 0 (no pain) to 10 (worst possible pain); a negative treatment difference (DMTS−Placebo) favors DMTS.
(2) Inferential statistics were from an ANCOVA model with treatment as independent variable, and clinical study unit as a covariate.

The NRSSPI over 5 to 96 hours after surgery of both sized DMTS groups (i.e., the 12 cm² DMTS group and the 15 cm² DMTS group) compared to placebo group was also evaluated using an analysis of covariance (ANCOVA) model. The results are shown in Table 14.

TABLE 14
NRSSPI from 5 hours through 96 hours after surgery for both
DMTS groups compared to placebo

| | NRSSPI Observed Data (ANCOVA Model) | | |
|---|---|---|---|
| | Least Squares Mean Treatment Difference | | |
| | [DMTS − placebo] (p-value) | | |
| | DMTS 12 cm² | DMTS 15 cm² | All DMTS |
| Hours After | (N = 25) vs. Placebo | (N = 57) vs. | (N = 82) vs. |
| Surgery | (N = 85) | Placebo (N = 85) | placebo (N = 85) |
| 5 to 6 Hours | −1.0 (p = 0.035) | −1.5 (p < 0.001) | −1.3 (p < 0.001) |
| 5 to 7 Hours | −1.7 (p = 0.056) | −2.6 (p < 0.001) | −2.4 (p < 0.001) |
| 5 to 8 Hours | −2.6 (p = 0.05) | −3.7 (p < 0.001) | −3.3 (p < 0.001) |
| 5 to 10 Hours | −5.1 (p = 0.015) | −6.2 (p < 0.001) | −5.8 (p < 0.001) |
| 5 to 12 Hours | −7.8 (p = 0.007) | −8.7 (p < 0.001) | −8.4 (p < 0.001) |
| 5 to 16 Hours | −11.4 (p = 0.012) | −13.4 (p < 0.001) | −12.8 (p < 0.001) |
| 5 to 24 Hours | −16.0 (p = 0.043) | −21.5 (p < 0.001) | −19.8 (p < 0.001) |
| 5 to 32 Hours | −20.3 (p = 0.068) | −27.2 (p = 0.001) | −25.1 (p = 0.001) |
| 5 to 40 Hours | −24.2 (p = 0.099) | −31.9 (p = 0.004) | −29.5 (p = 0.003) |
| 5 to 48 Hours | −28.4 (p = 0.117) | −36.8 (p = 0.007) | −34.3 (p = 0.006) |
| 5 to 56 Hours | −31.4 (p = 0.145) | −40.3 (p = 0.014) | −37.6 (p = 0.011) |
| 5 to 64 Hours | −33.3 (p = 0.183) | 44.2 (p = 0.02) | −40.9 (p = 0.017) |
| 5 to 72 Hours | −38.6 (p = 0.177) | −49.5 (p = 0.022) | −46.2 (p = 0.018) |
| 5 to 84 Hours | −43.6 (P = 0.196) | −57.3 (p = 0.024) | −53.1 (p = 0.021) |
| 5 to 96 Hours | 45.7 (p = 0.24) | −64.1 (p = 0.029) | −58.5 (p = 0.028) |

### 7.3 Use of Rescue Pain Medication

At all time intervals evaluated (0 to 5, 6, 7, 8, 10, 12, 16, 24, 32, 40, 48, 56, 64, 72, 84 and 96 hours after surgery), a similar proportion of subjects in the placebo and All DMTS groups used a rescue medication.

The morphine equivalent dose for use of the rescue medications of morphine and oxycodone was compared between treatment groups for the intervals from 0 to 5, 6, 7, 8, 10, 12, 16, 24, 32, 40, 48, 56, 64, 72, 84 and 96 hours after surgery. For each time interval, the All DMTS group had a significantly lower morphine equivalent dose than the placebo group (Table 15).

TABLE 15
Use of Rescue Analgesic Medications: Morphine and Oxycodone

| | Total Morphine Equivalent Dose (mg) | | | |
|---|---|---|---|---|
| | LS Mean (SE) | | LS Mean | |
| | All Placebo | All DMTS | Difference | |
| Timing | N = 85 | N = 82 | (95% CI) | p-value |
| 0 to 5 hours after surgery | 5.2 (2.08) | 4.0 (2.13) | −1.3 (−3, 0) | 0.0500 |
| 0 to 6 hours after surgery | 9.2 (2.13) | 7.7 (2.18) | −1.5 (−3, −0) | 0.0228 |
| 0 to 7 hours after surgery | 9.4 (2.25) | 7.7 (2.31) | −1.8 (−3, −0) | 0.0115 |
| 0 to 8 hours after surgery | 9.5 (2.26) | 7.6 (2.31) | −1.9 (−3, −0) | 0.0082 |
| 0 to 10 hours after surgery | 10.2 (2.79) | 7.5 (2.86) | −2.7 (−4, −1) | 0.0018 |
| 0 to 12 hours after surgery | 10.8 (3.10) | 7.4 (3.170 | −3.4 (−5, −1) | 0.0005 |
| 0 to 16 hours after surgery | 11.5 (3.63) | 7.3 (3.71) | −4.2 (−6, −2) | 0.0002 |
| 0 to 24 hours after surgery | 12.8 (4.46) | 6.9 (4.57) | −5.8 (−9, −3) | <0.0001 |
| 0 to 32 hours after surgery | 14.2 (5.54) | 6.8 (5.67) | −7.4 (−11, −4) | <0.0001 |
| 0 to 40 hours after surgery | 15.0 (6.35) | 6.9 (6.50) | −8.1 (−12, −4) | <0.0001 |
| 0 to 48 hours after surgery | 15.8 (7.21) | 7.1 (7.39) | −8.7 (−13, −4) | 0.0001 |
| 0 to 56 hours after surgery | 16.6 (7.90) | 7.0 (8.08) | −9.6 (−14, −5) | 0.0001 |
| 0 to 64 hours after surgery | 17.2 (8.96) | 7.1 (8.77) | −10.1 (−15, −5) | 0.0002 |
| 0 to 72 hours after surgery | 17.5 (9.15) | 7.4 (9.37) | −10.1 (−16, −5) | 0.0004 |
| 0 to 84 hours after surgery | 18.2 (10.20) | 7.5 (10.44) | −10.8 (−17, −5) | 0.0007 |
| 0 to 96 hours after surgery | 18.8 (11.23) | 7.6 (11.50) | −11.2 (−18. −4) | 0.0014 |

Inferential statistics were from an ANCOVA model with dose of rescue analgesic medications as the response variable, treatment (DMTS or placebo) as an independent variable, and clinical study unit as a covariate.

The ibuprofen dose was also compared between treatment groups and the time intervals noted above. The results (not shown) demonstrated that the All DMTS group had a significantly lower ibuprofen dose at each time interval compared with the placebo group.

The frequency of rescue analgesic medications (IV morphine, oral oxycodone, oral ibuprofen) was compared between treatment groups at time intervals from 0 to 5, 6, 7, 8, 10, 12, 16, 24, 32, 40, 48, 56, 64, 72, 84 and 96 hours after surgery. At all time intervals, the All DMTS group used rescue analgesia significantly less frequently than the placebo group (not shown).

As shown in Table 16, subjects in both DMTS groups requested rescue medications (ibuprofen, oxycodone, or morphine) less frequently compared to the placebo group from 0 to 96 hours after surgery.

TABLE 16
Frequency of rescue analgesia

| | Ibuprofen, Oxycodone and/or Morphine | | |
|---|---|---|---|
| | Least Squares Mean Difference [DMTS − placebo] (p-value) | | |
| | DMTS 12 cm² | DMTS 15 cm² | All DMTS vs. |
| Hours After Surgery | vs. placebo | vs. placebo | placebo |
| 0 to 12 Hours | −1.6 (p = 0.001) | −0.9 (p = 0.008) | −1.1 (p < 0.001) |
| 0 to 24 Hours | −2.3 (p < 0.001) | −1.5 (p = 0.002) | −1.8 (p < 0.001) |
| 0 to 48 Hours | −3.5 (p < 0.001) | −2.5 (p = 0.001) | −2.8 (p < 0.001) |
| 0 to 72 Hours | −4.5 (p = 0.001) | −3.0 (p = 0.002) | −3.4 (p < 0.001) |
| 0 to 96 Hours | −5.1 (p = 0.001) | −3.3 (p = 0.006) | −3.8 (p < 0.001) |

Subjects in the 12 cm² DMTS group requested rescue medications less frequently compared to the placebo group than subjects in the 15 cm² DMTS group compared to the placebo group.

As shown in Table 17, the proportion of subjects in the DMTS groups that used rescue oxycodone was lower than the proportion of subjects that used rescue oxycodone in the placebo group during both time intervals (0 to 12 hours after surgery and >12 hours after surgery). Surprisingly, during both time intervals, the proportion of subjects in the 12 cm² DMTS group that used rescue oxycodone compared to the placebo group was less than the proportion of subjects in the 15 cm² DMTS group that used rescue oxycodone compared to the placebo group.

TABLE 17
Proportion of subjects using oxycodone rescue analgesia

| Proportion of Subjects Using Oxycodone | | | |
|---|---|---|---|
| Placebo | DMTS 12 cm² | DMTS 15 cm² | All DMTS |
| (N = 85) | (N = 25) | (N = 57) | (N = 82) |

0 to 12 Hours After Surgery

| | | | | |
|---|---|---|---|---|
| Yes, n (%) | 47 (55.3%) | 5 (20%) | 18 (31.6%) | 23 (28.0%) |
| Proportion Difference | | −35.3% | −23.7% | −27.3% |
| [DMTS − Placebo] (p-value) | | (p = 0.005) | (p = 0.006) | (p < 0.001) |

>12 Hours After Surgery

| | | | | |
|---|---|---|---|---|
| Yes, n (%) | 48 (56.5%) | 8 (32%) | 23 (40.4%) | 31 (37.8%) |
| Proportion Difference | | −24.5% | −16.1% | −18.7% |
| (DMTS − Placebo] (p-value) | | (p = 0.033) | (p = 0.064) | (p = 0.016) |

As shown in Table 18, the total dose of rescue opioid (oxycodone and/or morphine) used in the DMTS groups was lower than the total does of rescue opioid used in the placebo group from 0 to 96 hours after surgery. Surprisingly, subjects in the 12 cm² DMTS group used less rescue opioid compared to the placebo group than subjects in the 15 cm² DMTS group compared to the placebo group.

TABLE 18
Total dose of rescue opioid (mg oxycodone and/or morphine)

| | Oxycodone and/or Morphine | | |
|---|---|---|---|
| | (in Morphine Equivalent Dose, mg) Least | | |
| | Squares Mean Difference (p-value) | | |
| Hours After | DMTS 12 cm² vs. | DMTS 15 cm² vs. | All DMTS vs. |
| Surgery | placebo | Placebo | placebo |
| 0 to 12 Hours | −4.8 (p = 0.001) | −2.7 (p = 0.011) | −3.4 (p = 0.001) |
| 0 to 24 Hours | −8.2 (p < 0.001) | −4.8 (p = 0.002) | −5.8 (p < 0.001) |
| 0 to 48 Hours | −11.7 (p < 0.001) | −7.5 (p = 0.003) | −8.7 (p < 0.001) |
| 0 to 72 Hours | −13.8 (p = 0.001) | −8.4 (p = 0.007) | −10.1 (p < 0.001) |
| 0 to 96 Hours | −15.1 (p = 0.003) | −9.5 (p = 0.014) | −11.2 (p = 0.001) |

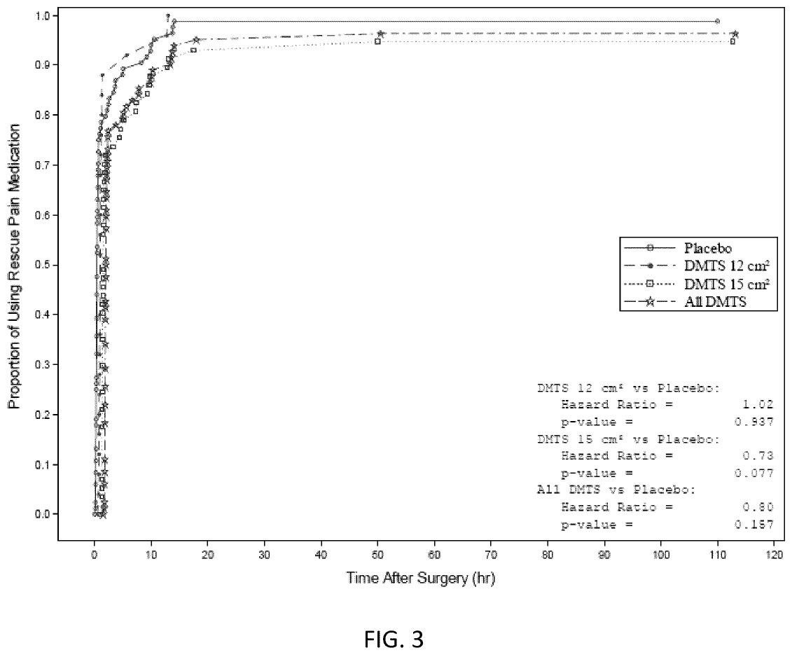

The time to first use of rescue analgesic medication (morphine, oxycodone, or ibuprofen) is depicted in FIG. 3. The Kaplan-Meier estimate of the median time to the first use of a rescue analgesic medication was similar between the placebo group and the DMTS group (0.4 hours and 0.5 hours, respectively; p=0.157).

An analysis of comparing between treatment groups the NRSSPI at the time intervals of 1 to 2, 3, 4, 5, 6, 7, 8, 10, 12, 16, 24, 32, 40, 48, 56, 64, 72, 84 and 96 hours after surgery was performed. This analysis showed that the All DMTS group had statistically significantly lower NRSSPI scores within each time interval than the placebo group. As shown in Table 19, subjects in the DMTS groups had longer time until first use of rescue oxycodone after surgery compared to the placebo group. Surprisingly, subjects in the 12 cm² DMTS group waited longer to request rescue medication than subjects in the 15 cm² DMTS group.

TABLE 19
Time to first use of rescue oxycodone

| | Time (hrs) to First Use of Oxycodone | | | |
|---|---|---|---|---|
| | | DMTS | DMTS | |
| | Placebo | 12 cm² | 15 cm² | All DMTS |
| | (N = 85) | (N = 25) | (N = 57) | (N = 82) |
| Event, n | 62 | 9 | 28 | 37 |
| Median Time (hrs) to | 10.6 | 42.5 * | 21.7 * | 42.5 * |
| First Use (p-value) | | (p = 0.001) | (p = 0.002) | (p < 0.0001) |

### 7.4 Integrated Variable

An integrated variable incorporating both pain scores (NRSSPI) and rescue analgesic medications (IV morphine, oral oxycodone, oral ibuprofen) was determined. The integrated assessment summed the % difference from the mean rank in pain scores and rescue mediation use.

% difference from the mean rank=(rank for each subject−mean rank amount of the overall population)/mean rank among the overall population

Analysis of the integrated variable over 5 to 6, 7, 8, 10, 12, 16, 24, 32, 40, 48, 56, 64, 72, 84 and 96 hours after surgery showed a statistically significant difference between treatment groups at each time interval (Table 20).

TABLE 20
Integrated assessment of pain scores measured at rest and rescue analgesic
medications over time (ITT Analysis Population)

| | | | | |
|---|---|---|---|---|
| Summed % | All Placebo | DMTS 12 cm² | DMTS 15 cm² | All DMTS |
| Difference | N = 85 | (N = 25) | (N = 57) | (N = 82) |

5 to 6 Hours After Surgery

| | | | | |
|---|---|---|---|---|
| n | 84 | 25 | 57 | 82 |
| Mean (SD) | −30.1 (86.96) | 40.0 (76.88) | 26.9 (93.47) | 30.9 (88.48) |
| Median | −39.8 | 34.1 | 22.8 | 26.0 |
| Range (min, max) | (−195, 176) | (−113, 178) | (−171, 186) | (−171, 186) |
| DMTS vs. | | 0.0006 | 0.0006 | <0.0001 |
| Placebo p-valueª | | | | |

5 to 7 Hours After Surgery

| | | | | |
|---|---|---|---|---|
| n | 84 | 25 | 57 | 82 |
| Mean (SD) | −28.8 (86.75) | 41.1 (78.94) | 24.4 (98.52) | 29.5 (92.83) |
| Median | −29.9 | 30.5 | 25.1 | 28.7 |
| Range (min, max) | (−193, 178) | (−114, 165) | (−168, 189) | (−168, 189) |
| DMTS vs. | | 0.0009 | 0.0016 | <0.0001 |
| Placebo p-valueª | | | | |

5 to 8 Hours After Surgery

| | | | | |
|---|---|---|---|---|
| n | 84 | 25 | 57 | 82 |
| Mean (SD) | −28.6 (88.03) | 43.5 (78.26) | 23.1 (100.66) | 29.3 (94.39) |
| Median | −25.4 | 41.3 | 31.7 | 33.8 |
| Range (min, max) | (−194, 179) | (−120, 163) | (−165, 189) | (−165, 189) |
| DMTS vs. | | 0.00008 | 0.0035 | 0.0002 |
| Placebo p-valueª | | | | |

5 to 10 Hours After Surgery

| | | | | |
|---|---|---|---|---|
| n | 84 | 25 | 57 | 82 |
| Mean (SD) | −32.0 (88.73) | 49.7 (79.07) | 25.3 (99.36) | 32.8 (93.84) |
| Median | −24.0 | 83.2 | 34.7 | 47.0 |
| Range (min, max) | (−196, 182) | (−165, 166) | (−150, 190) | (−165, 190) |
| DMTS vs. | | 0.0001 | 0.0010 | <0.0001 |
| Placebo p-valueª | | | | |

5 to 12 Hours After Surgery

| | | | | |
|---|---|---|---|---|
| n | 84 | 25 | 57 | 82 |
| Mean (SD) | −35.1 (87.69) | 52.3 (73.77) | 28.8 (99.44) | 36.0 (92.56) |
| Median | −38.6 | 64.6 | 34.7 | 46.7 |
| Range (min, max) | (−195, 181) | (−154, 165) | (−169, 191) | (−169, 191) |
| DMTS vs. | | <0.0001 | 0.0002 | <0.0001 |
| Placebo p-valueª | | | | |

5 to 16 Hours After Surgery

| | | | | |
|---|---|---|---|---|
| n | 84 | 25 | 57 | 82 |
| Mean (SD) | −35.1 (87.81) | 51.9 (72.41) | 29.0 (98.24) | 36.0 (91.31) |
| Median | −52.1 | 55.1 | 49.1 | 51.8 |
| Range (min, max) | (−193, 169) | (−128, 160) | (−176, 192) | (−176, 192) |
| DMTS vs. | | <0.0001 | 0.0001 | <0.0001 |
| Placebo p-valuea | | | | |

5 to 24 Hours After Surgery

| | | | | |
|---|---|---|---|---|
| n | 84 | 25 | 57 | 82 |
| Mean (SD) | −34.9 (89.63) | 46.9 (70.33) | 27.4 (93.36) | 33.4 (87.02) |
| Median | −47.6 | 61.7 | 34.7 | 45.2 |
| Range (min, max) | (−198, 177) | (−101, 153) | (−162, 189) | (−162, 189) |
| DMTS vs. | | <0.0001 | 0.0002 | <0.0001 |
| Placebo p-valueª | | | | |

5 to 32 Hours After Surgery

| | | | | |
|---|---|---|---|---|
| n | 84 | 25 | 57 | 82 |
| Mean (SD) | −33.4 (88.04) | 48.8 (74.83) | 27.8 (94.26) | 34.2 (88.86) |
| Median | −45.2 | 76.7 | 31.7 | 42.2 |
| Range (min, max) | (−190, 177) | (−121, 156) | (−167, 189) | (−167, 189) |
| DMTS vs. | | <0.0001 | 0.0003 | <0.0001 |
| Placebo p-valueª | | | | |

5 to 40 Hours After Surgery

| | | | | |
|---|---|---|---|---|
| n | 84 | 25 | 57 | 82 |
| Mean (SD) | −31.7 (87.93) | 45.7 (78.01) | 26.7 (95.52) | 32.5 (90.49) |
| Median | −36.8 | 74.9 | 25.7 | 44.3 |
| Range (min, max) | (−192, 178) | (−123, 152) | (−159, 189) | (−159, 189) |
| DMTS vs. | | 0.0001 | 0.0006 | <0.0001 |
| Placebo p-valuea | | | | |

5 to 48 Hours After Surgery

| | | | | |
|---|---|---|---|---|
| n | 84 | 25 | 57 | 82 |
| Mean (SD) | −30.4 (88.60) | 43.8 (77.38) | 25.6 (96.12) | 31.2 (90.74) |
| Median | −41.3 | 56.3 | 28.7 | 44.0 |
| Range (min, max) | (−192, 175) | (−127, 154) | (−160, 185) | (−160, 185) |
| DMTS vs. | | 0.0003 | 0.0007 | <0.0001 |
| Placebo p-valueª | | | | |

5 to 56 Hours After Surgery

| | | | | |
|---|---|---|---|---|
| n | 84 | 25 | 57 | 82 |
| Mean (SD) | −29.2 (88.14) | 42.7 (78.73) | 24.3 (97.31) | 29.9 (91.95) |
| Median | −39.5 | 56.9 | 22.2 | 35.9 |
| Range (min, max) | (−190, 176) | (−132, 156) | (−160, 185) | (−160, 185) |
| DMTS vs. | | 0.0005 | 0.0015 | <0.0001 |
| Placebo p-valueª | | | | |

5 to 64 Hours After Surgery

| | | | | |
|---|---|---|---|---|
| n | 84 | 25 | 57 | 82 |
| Mean (SD) | −27.6 (88.24) | 40.4 (77.81) | 22.9 (98.59) | 28.2 (92.62) |
| Median | −42.8 | 50.3 | 10.2 | 26.9 |
| Range (min, max) | (−191, 178) | (−123, 156) | (−156, 185) | (−156, 185) |
| DMTS vs. | | 0.0008 | 0.0024 | 0.0001 |
| Placebo p-valuea | | | | |

5 to 72 Hours After Surgery

| | | | | |
|---|---|---|---|---|
| n | 84 | 25 | 57 | 82 |
| Mean (SD) | −29.6 (89.29) | 40.9 (75.47) | 20.3 (99.76( | 26.6 (93.06) |
| Median | −41.9 | 53.9 | 13.2 | 32.6 |
| Range (min, max) | (−192, 174) | (−107, 154) | (−159, 182) | (0159, 182) |
| DMTS vs. | | 0.0004 | 0.0031 | 0.0001 |
| Placebo p-valueª | | | | |

5 to 84 Hours After Surgery

| | | | | |
|---|---|---|---|---|
| n | 84 | 25 | 57 | 82 |
| Mean (SD) | −26.6 (89.99) | 39.1 (76.18) | 22.1 (99.90) | 27.2 (93.17) |
| Median | −32.9 | 34.1 | 20.4 | 21.6 |
| Range (min, max) | (−190, 174) | (−105, 159) | (−157, 184) | (−157, 184) |
| DMTS vs. | | 0.0010 | 0.0055 | 0.0003 |
| Placebo p-valueª | | | | |

5 to 96 Hours After Surgery

| | | | | |
|---|---|---|---|---|
| n | 84 | 25 | 57 | 82 |
| Mean (SD) | −27.7 (90.18) | 35.7 (78.56) | 21.0 (100.62) | 25.5 (94.21) |
| Median | −32.2 | 31.7 | 12.6 | 24.3 |
| Range (min, max) | (−187, 171) | (−124, 161) | (−162, 183) | (−162, 183) |
| DMTS vs. | | 0.0020 | 0.0050 | 0.0004 |
| Placebo p-valueª | | | | |

^{a}p-value was from Wilcoxon rank-sum test.

Sensitivity analyses of the integrated variable using LOCF or WOCF imputation for pain assessments collected during the efficacious windows of the rescue medications were performed. The results of these analyses (not shown) also indicated a statistically significant difference between treatment group at each time interval.

The integrated assessment of pain score and rescue analgesia for both DMTS groups and the placebo group was calculated (Table 21).

TABLE 21
Integrated assessment of pain scores and rescue medication use

| | Integrated Assessment of Pain Scores Measured at | | | |
|---|---|---|---|---|
| | Rest and Rescue Analgesic Medications | | | |
| | Median (p-value, DMTS vs. placebo) | | | |
| Hours After | Placebo | DMTS 12 cm² | DMTS 15 cm² | All DMTS |
| Surgery | (N = 85) | (N = 25) | (N = 57) | (N = 82) |
| 5 to 12 Hours | −38.6 | 64.7 (p < 0.001) | 34.7 (p < 0.001) | 46.7 (p < 0.001) |
| 5 to 24 Hours | −34.9 | 46.9 (p < 0.001) | 27.4 (p < 0.001) | 33.4 (p < 0.001) |
| 5 to 48 Hours | −41.3 | 56.3 (p < 0.001) | 28.7 (p = 0.001) | 44 (p < 0.001) |
| 5 to 72 Hours | −41.9 | 53.9 (p < 0.001) | 13.2 (p = 0.003) | 32.6 (p < 0.001) |
| 5 to 96 Hours | −32.3 | 31.7 (p = 0.002) | 12.6 (p = 0.005) | 24.3 (p < 0.001) |

As shown in Table 21, subjects in the DMTS groups had a statistically better integrated assessment than the placebo group from 0 to 96 hours after surgery due to the combined pain relief and reduced used of analgesic medications of DMTS. Surprisingly, subjects in the 12 cm² DMTS group had greater integrated assessments than the subjects in the 15 cm² DMTS group.

### 7.5 Pharmacokinetics

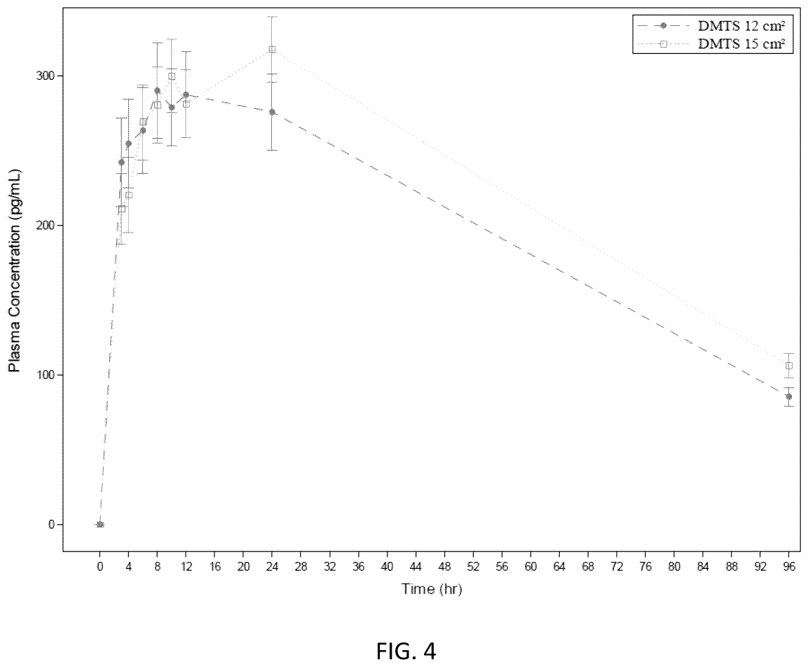

Dexmedetomidine plasma concentrations over time are depicted in FIG. 4.

Dexmedetomidine PK parameters are summarized for the PK/PD Analysis Population in Table 22. For subjects who received treatment with DMTS 12 cm² (containing 2.92 mg of dexmedetomidine), the geometric mean Cₘₐₓ was 304.1 pg/mL, and the geometric mean AUC₅₋₉₆ was 17062.4 h×pg/mL.

For subjects who received treatment with DMTS 15 cm² (containing 3.65 mg of dexmedetomidine), the geometric mean Cₘₐₓ was 301.1 pg/mL, and the geometric mean AUC₅₋₉₆ was 15810.9 h×pg/mL.

The AUC₅₋₉₆ and Tₘₐₓ values are expected to increase proportionally to the dose or the patch size. Due to the large intersubject variability and small sample size, the experimentally derived values are not always related as expected.

TABLE 22
Dexmedetomidine Pharmacokinetic Parameters for
Subjects Treated with DMTS

| | |
|---|---|
| AUC₅₋₉₆ (h × pg/mL) | Cₘₐₓ (pg/mL) |

DMTS 12 cm²

| | | |
|---|---|---|
| N | 25 | 25 |
| Mean (SD) | 18418.3 (7295.70) | 337.1 (149.64) |
| Median | 17591.3 | 340.0 |
| Min, Max | (8788, 34033) | (131, 707) |
| % CV | 39.6 | 44.4 |
| Geometric Mean (SD) | 17062.4 (1.50) | 304.1 (1.61) |
| % Geometric CV | 42.1 | 50.8 |

DMTS 15 cm²

| | | |
|---|---|---|
| N | 52 | 57 |
| Mean (SD) | 19741.6 (9901.62) | 350.8 (187.43) |
| Median | 18017.8 | 316.0 |
| Min, Max | (106, 54087) | (38, 877) |
| % CV | 50.2 | 53.4 |
| Geometric Mean (SD) | 15810.9 (2.59) | 301.1 (1.82) |
| % Geometric CV | 121.5 | 65.5 |

AUC₅₋₉₆ was compared between subgroups of sex (male vs female), race (White vs Non-White), age (≤the median of 38 years vs >the median of 38 years), body weight (≤the median of 77.7 kg vs >the median of 77.7 kg), patch/system size (15 cm² vs 12 cm²). For each analysis, the AUC₅₋₉₆ was similar for the subgroups.

The Pearson correlation coefficient for the dexmedetomidine plasma concentration AUC and the NRSSPI was determined for time intervals of 5 to 24, 48, 72, and 96 hours after surgery. The correlation coefficients were −0.015, 0.033, 0.044, and 0.015, respectively, none of which was statistically significant (p=0.900, 0.778, 0.707, and 0.899 for the 4 comparisons, coefficients, respectively).

### 7.6 Pharmacodynamics

Sedation levels, evaluated using the Wilson sedation scale (Table 7), are summarized by treatment group in Table 23. At all assessment time points, most subjects had a Wilson sedation score of 1 (fully awake and oriented). Sedation scores were consistent across treatment groups. No subject in either treatment group (placebo or DMTS) at any assessment time point had a sedation score of 4 (eyes closed but rousable to mild physical stimulation) or 5 (eyes closed and unrousable to mild physical stimulation).

TABLE 23
Sedation Level

| | All | DMTS | | | |
|---|---|---|---|---|---|
| | Placebo | 12 cm² | 15 cm² | All | Overall |
| | N = 85 | N = 25 | N = 57 | N = 82 | N = 167 |
| Statistics | n (%) | n (%) | n (%) | n (%) | n (%) |
| Day 2 | 67 (78.8) | 21 (84.0) | 46 (80.7) | 67 (81.7) | 134 (80.2) |
| (1) Fully awake and oriented | 54 (63.5) | 19 (76.0) | 35 (61.4) | 54 (65.9) | 108 (64.7) |
| (2) Drowsy | 9 (10.6) | 2 (8.0) | 5 (8.8) | 7 (8.5) | 16 (9.6) |
| (3) Eyes closed but rousable | 4 (4.7) | 0 (0.0) | 6 (10.5) | 6 (7.3) | 10 (6.0) |
| to command | | | | | |
| (4) Eyes closed but rousable | 0 (0.0) | 0 (0.0) | 0 (0.0) | 0 (0.0) | 0 (0.0) |
| to mild physical stimulation | | | | | |
| (earlobe tug) | | | | | |
| (5) Eyes closed but rousable | 0 (0.0) | 0 (0.0) | 0 (0.0) | 0 (0.0) | 0 (0.0) |
| to mild physical stimulation | | | | | |
| Day 3 | 68 (80.0) | 21 (84.0) | 47 (82.5) | 68 (82.9) | 136 (81.4) |
| (1) Fully awake and oriented | 62 (72.9) | 20 (80.0) | 41 (71.9) | 61 (74.4) | 123 (73.7) |
| (2) Drowsy | 2 (2.4) | 1 (4.0) | 5 (8.8) | 6 (7.3) | 8 (4.8) |
| (3) Eyes closed but rousable | 4 (4.7) | 0 (0.0) | 1 (1.8) | 1 (1.2) | 5 (3.0) |
| to command | | | | | |
| (4) Eyes closed but rousable | 0 (0.0) | 0 (0.0) | 0 (0.0) | 0 (0.0) | 0 (0.0) |
| to mild physical stimulation | | | | | |
| (earlobe tug) | | | | | |
| (5) Eyes closed but rousable | 0 (0.0) | 0 (0.0) | 0 (0.0) | 0 (0.0) | 0 (0.0) |
| to mild physical stimulation | | | | | |
| Day 4 | 68 (80.0) | 20 (80.0) | 43 (75.4) | 63 (76.8) | 131 (78.4) |
| (1) Fully awake and oriented | 62 (72.9) | 20 (80.0) | 41 (71.9) | 61 (74.4) | 123 (73.7) |
| (2) Drowsy | 6 (7.1) | 0 (0.0) | 2 (3.5) | 2 (2.4) | 8 (4.8) |
| (3) Eyes closed but rousable | 0 (0.0) | 0 (0.0) | 0 (0.0) | 0 (0.0) | 0 (0.0) |
| to command | | | | | |
| (4) Eyes closed but rousable | 0 (0.0) | 0 (0.0) | 0 (0.0) | 0 (0.0) | 0 (0.0) |
| to mild physical stimulation | | | | | |
| (earlobe tug) | | | | | |
| (5) Eyes closed but rousable | 0 (0.0) | 0 (0.0) | 0 (0.0) | 0 (0.0) | 0 (0.0) |
| to mild physical stimulation | | | | | |
| Day 5 | 64 (75.3) | 20 (80.0) | 41 (71.91) | 61 (74.4) | 125 (74.9) |
| (1) Fully awake and oriented | 62 (72.9) | 20 (80.0) | 40 (70.2) | 60 (73.2) | 122 (73.1) |
| (2) Drowsy | 1 (1.2) | 0 (0.0) | 0 (0.0) | 0 (0.0) | 0 (0.0) |
| (3) Eyes closed but rousable | 1 (1.2) | 0 (0.0) | 1 (1.8) | 1 (1.2) | 1 (1.2) |
| to command | | | | | |
| (4) Eyes closed but rousable | 0 (0.0) | 0 (0.0) | 0 (0.0) | 0 (0.0) | 0 (0.0) |
| to mild physical stimulation | | | | | |
| (earlobe tug) | | | | | |
| (5) Eyes closed but rousable | 0 (0.0) | 0 (0.0) | 0 (0.0) | 0 (0.0) | 0 (0.0) |
| to mild physical stimulation | | | | | |
| Day 6 | 52 (61.2) | 14 (56.0) | 38 (66.7) | 52 (63.4) | 104 (62.3) |
| (1) Fully awake and oriented | 52 (61.2) | 14 (56.0) | 34 (59.6) | 48 (58.5) | 100 (59.9) |
| (2) Drowsy | 0 (0.0) | 0 (0.0) | 3 (5.3) | 3 (3.7) | 3 (1.8) |
| (3) Eyes closed but rousable | 0 (0.0) | 0 (0.0) | 1 (1.8) | 1 (1.2) | 1 (0.6) |
| to command | | | | | |
| (4) Eyes closed but rousable | 0 (0.0) | 0 (0.0) | 0 (0.0) | 0 (0.0) | 0 (0.0) |
| to mild physical stimulation | | | | | |
| (earlobe tug) | | | | | |
| (5) Eyes closed but rousable | 0 (0.0) | 0 (0.0) | 0 (0.0) | 0 (0.0) | 0 (0.0) |
| to mild physical stimulation | | | | | |
| End of Study | 62 (72.9) | 20 (80.0) | 46 (80.7) | 66 (80.5) | 128 (76.6) |
| (1) Fully awake and oriented | 61 (71.8) | 20 (80.0) | 46 (80.7) | 66 (80.5) | 127 (76.0) |
| (2) Drowsy | 1 (1.2) | 0 (0.0) | 0 (0.0) | 0 (0.0) | 0 (0.0) |
| (3) Eyes closed but rousable | 0 (0.0) | 0 (0.0) | 0 (0.0) | 0 (0.0) | 0 (0.0) |
| to command | | | | | |
| (4) Eyes closed but rousable | 0 (0.0) | 0 (0.0) | 0 (0.0) | 0 (0.0) | 0 (0.0) |
| to mild physical stimulation | | | | | |
| (earlobe tug) | | | | | |
| (5) Eyes closed but rousable | 0 (0.0) | 0 (0.0) | 0 (0.0) | 0 (0.0) | 0 (0.0) |
| to mild physical stimulation | | | | | |

Sedation was assessed using the Wilson Sedation Scale. Sedation data collected after the treatment interruption or early discontinuation were excluded from the summary.

### 7.7 Adverse Events

A summary of the AEs is provided in Table 24.

TABLE 24
Adverse events

| | All placebo | DMTS 12 cm² | DMTS 15 cm² | All DMTS |
|---|---|---|---|---|
| | N = 85 | N = 25 | N = 57 | N = 82 |
| | n (%) | n (%) | n (%) | n (%) |
| Any AEs | 5 (5.9) | 3 (12.0) | 3 (5.3) | 6 (7.3) |
| Treatment-Emergent AEs | 81 (95.3) | 22 (88.0) | 55 (96.5) | 77 (93.9) |
| Treatment-Emergent AEs Related | 26 (30.6) | 13 (52.0) | 32 (56.1) | 45 (54.9) |
| to Study Drug | | | | |
| Mild | 18 (21.2) | 7 (28.0) | 20 (35.1) | 27 (32.9) |
| Moderate | 8 (9.4) | 6 (24.0) | 12 (21.1) | 18 (22.0) |
| Severe | 0 (0.0) | 0 (0.0) | 0 (0.0) | 0 (0.0) |
| Treatment-Emergent Serious AEs | 1 (1.2) | 0 (0.0) | 0 (0.0) | 0 (0.0) |
| Treatment-Emergent Serious AEs | 0 (0.0) | 0 (0.0) | 0 (0.0) | 0 (0.0) |
| Related to Study Drug | | | | |
| Mild | 0 (0.0) | 0 (0.0) | 0 (0.0) | 0 (0.0) |
| Moderate | 0 (0.0) | 0 (0.0) | 0 (0.0) | 0 (0.0) |
| Severe | 0 (0.0) | 0 (0.0) | 0 (0.0) | 0 (0.0) |
| Subjects who died - (placebo, | 1 (1.2) | 0 (0.0) | 0 (0.0) | 0 (0.0) |
| pulmonary embolism) | | | | |
| Withdrawn Due to | | | | |
| Treatment-Emergent AEs | 1 (1.2) | 0 (0.0) | 1 (1.8) | 1 (1.2) |
| Treatment-Emergent AEs Related | 1 (1.2) | 0 (0.0) | 1 (1.8) | 1 (1.2) |
| to Study Drug | | | | |

A summary of treatment-emergent AEs which occurred in >5% of subjects is provided in Table 25.

TABLE 25
Treatment-emergent AEs occurring in >5% of subjects

| | All placebo | DMTS 12 cm² | DMTS 15 cm² | All DMTS |
|---|---|---|---|---|
| | N = 85 | N = 25 | N = 57 | N = 82 |
| Preferred Term | n (%) | n (%) | n (%) | n (%) |
| Nausea | 59 (69.4) | 13 (52.0) | 37 (64.9) | 50 (61.0) |
| Constipation | 32 (37.6) | 5 (20.0) | 18 (31.6) | 23 (28.0) |
| Hypoxia | 22 (25.9) | 7 (28.0) | 19 (33.3) | 26 (31.7) |
| Vomiting | 20 (23.5) | 2 (8.0) | 10 (17.5) | 12 (14.6) |
| Hypotension | 4 (4.7) | 11 (44.0) | 12 (21.1) | 23 (28.0) |
| Infusion site extravasation | 9 (10.6) | 1 (4.0) | 10 (17.5) | 11 (13.4) |
| Dizziness | 5 (5.9) | 0 (0.0) | 12 (21.1) | 12 (14.6) |
| Headache | 11 (12.9) | 1 (4.0) | 5 (8.8) | 6 (7.3) |
| Bradycardia | 4 (4.7) | 2 (8.0) | 5 (8.8) | 7 (8.5) |
| Application site erythema | 1 (1.2) | 0 (0.0) | 9 (15.8) | 9 (11.0) |
| Hepatic enzyme increased | 5 (5.9) | 1 (4.0) | 2 (3.5) | 3 (3.7) |
| Hypertension | 6 (7.1) | 1 (4.0) | 0 (0.0) | 1 (1.2) |
| Urinary retention | 2 (2.4) | 0 (0.0) | 4 (7.0) | 4 (4.9) |
| Rash | 1 (1.2) | 0 (0.0) | 3 (5.3) | 3 (3.7) |

Treatment-Emergent AEs (TEAE) were similar between the DMTS groups and placebo groups. Most AEs were mild or moderate in severity; no subject had a severe treatment related. One serious AE occurred in placebo group. Most subjects in both treatment groups (95.3% in the placebo group, 88.0% in the DMTS 12 cm² group, and 96.5% in the DMTS 15 cm² group) had a TEAE. TEAEs were considered by the investigator as related to study drug for 30.6% of subjects in the placebo group, 52.0% of subjects in the DMTS 12 cm² group, and 56.1% of subjects in the DMTS 15 cm² group. One subject (in the placebo group) had a fatal SAE (post procedural pulmonary embolism). Drug was withdrawn due to a TEAE for 1 subject (1.2%) in the placebo group and 1 subject (1.8%) in the DMTS 15 cm² group.

Most subjects had at least 1 TEAE during the study: 95.3% in the placebo group, 88.0% in the DMTS 12 cm² group, and 96.5% in the DMTS 15 cm² group. The MedDRA SOCs with the highest reported incidence of TEAEs (>25% in any treatment group) were gastrointestinal disorder (80.0% placebo, 68.0% DMTS 12 cm², 77.2% DMTS 15 cm²), respiratory, thoracic and mediastinal disorders (29.4% placebo, 28.0% DMTS 12 cm², 38.6% DMTS 15 cm²), general disorders and administration site conditions (22.4% placebo, 24.0% DMTS 12 cm², 42.1% DMTS 15 cm²), vascular disorders (15.3% placebo, 48.0% DMTS 12 cm², 29.8% DMTS 15 cm²), and nervous system disorders (22.4% placebo, 8.0% DMTS 12 cm², 31.6% DMTS 15 cm²).

By MedDRA PT, frequently reported TEAEs (>25% incidence in any treatment group) were nausea (69.4% placebo, 52.0% DMTS 12 cm², 64.9% DMTS 15 cm²), constipation (37.6% placebo, 20.0% DMTS 12 cm², 31.6% DMTS 15 cm²), hypoxia (25.9% placebo, 28.0% DMTS 12 cm², 33.3% DMTS 15 cm²), and hypotension (4.7% placebo, 44.0% DMTS 12 cm², 21.1% DMTS 15 cm²).

Notably, the DMTS groups experienced less constipation than the placebo group, likely due to the decreased use of rescue opioids in the DMTS groups.

TEAEs were considered by the investigator as related to study drug for 30.6%, 52.0%, and 56.1% of subjects in the placebo, DMTS 12 cm², and DMTS 15 cm² groups, respectively. TEAEs most frequently considered treatment-related were hypotension (4.7% placebo, 40.0% DMTS 12 cm², 21.1% DMTS 15 cm²), hypoxia (8.2% placebo, 4.0% DMTS 12 cm², 14.0% DMTS 15 cm²), dizziness (3.5% placebo, 0.0% DMTS 12 cm², 19.3% DMTS 15 cm²), and application site erythema (1.2% placebo, 0.0% DMTS 12 cm², 15.8% DMTS 15 cm²).

The severe post procedural pulmonary embolism (reported for 1 placebo subject) was the only reported SAE. The event had a fatal outcome and was considered by the investigator as not related to study treatment. Two subjects (1 placebo, 1 DMTS 15 cm²) each had symptomatic hypotension that led to early discontinuation of treatment.

In general, laboratory parameters and ECGs remained stable over the course of the study and changes were generally consistent between treatment groups. Changes in vital signs following application of DMTS/matching placebo showed decreases in systolic and diastolic BP for the 2 DMTS cohorts that were not observed in the placebo group. The decreases in BP reached a nadir at 24 hours after DMTS application, which returned toward baseline thereafter.

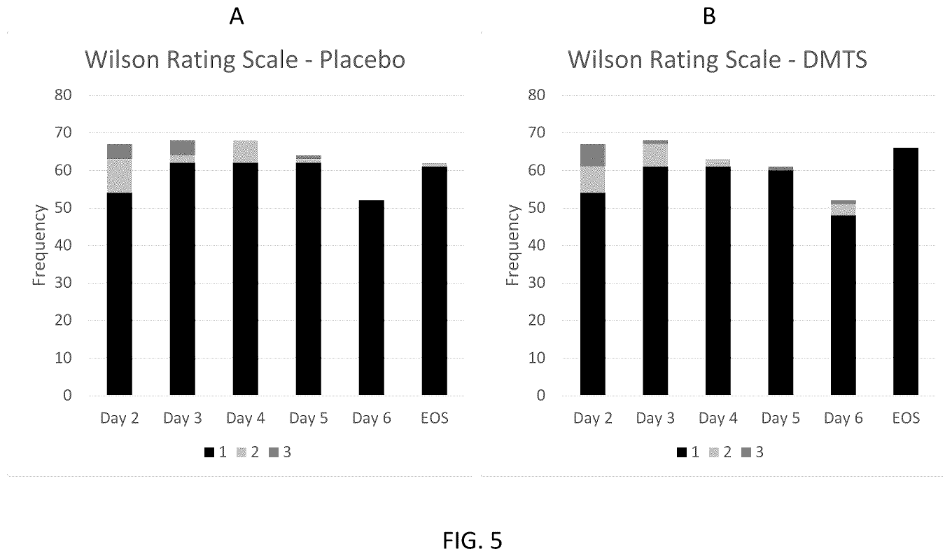

Sedation was acceptable, i.e., all subjects had a Wilson Rating Score of 3 or less, with the majority having a Wilson Rating Score of 1 (FIG. 5).

Skin irritation assessments showed that following removal DMTS/matching placebo, minimal/mild erythema, moderate erythema, minimal/mild papules, and vesicles were observed for a small proportion of subjects. No subject had edema. Erythema was observed as follows: 1 hour after DMTS/placebo removal, minimal/mild erythema was observed for 3 subjects (1.2%) in the placebo group and 6 subjects (3.5%) in the DMTS 15 cm² group; moderate erythema was observed for 2 subjects (1.2%) in the DMTS 15 cm² group. By 24 hours after DMTS/placebo removal, no subject in the placebo group had erythema; 7 subjects (4.1%) in the DMTS 15 cm² group had minimal/mild erythema, and 3 subject (1.8%) had moderate erythema. No subject in any treatment group had severe erythema at either assessment time point. The mean (SD) worst erythema score was 0.0 (0.11) in the placebo group, 0.0 (0.00) in the DMTS 12 cm² group, and 0.2 (0.44) in the DMTS 15 cm² group.

Papules were observed as follows: 1 hour after DMTS/placebo removal, 3 subjects (1.2%) in the placebo group and no subject in either DMTS group had papules; 24 hours after DMTS/placebo removal, 3 subjects (1.2%) in the placebo group and 1 subject (0.6%) in the DMTS 15 cm² group had minimal/mild papules. No subject in any treatment group had moderate or severe papules at either assessment time point. The mean (SD) worst papule score was 0.0 (0.11) in the placebo group, 0.0 (0.00) in the DMTS 12 cm² group, and 0.0 (0.04) in the DMTS 15 cm² group.

Vesicles were observed as follows: 1 hour after DMTS/placebo removal, no subject in any cohort had vesicles. At 24 hours after DMTS/placebo removal, 1 subject (0.4%) in the placebo group and no subject in either DMTS group had vesicles. The mean (SD) worst vesicle score was 0.0 (0.04) in the placebo group and 0.0 (0.00) in both DMTS groups.

The mean (SD) total worst irritation scores, determined across all skin irritation scores, were 0.0 (0.23) in the placebo group, 0.0 (0.00) in the DMTS 12 cm² group, and 0.2 (0.44) in the DMTS 15 cm² group.

### 7.8 Summary and Conclusions

The efficacy results showed that in subjects undergoing abdominoplasty, subjects who received DMTS (All DMTS group) had significantly less post-surgical pain as measured by the NRSSPI collected over 5 to 96 hours after surgery: the LS mean NRSSPI was 500.9 for the placebo group and 442.4 for the All DMTS group, with a treatment difference of −58.5 (95% CI: −111, −6; p=0.0280). Evaluation of the NRSSPI over the time intervals of 5 to 6, 7, 8, 10, 12, 16, 24, 32, 40, 48, 56, 64, 72, 84 and 96 hours after surgery showed that subjects in the All DMTS group had statistically significantly lower NRSSPI scores at each time interval than the placebo group.

While the proportion of subjects using rescue analgesic medications at all evaluated time intervals (0 to 5, 6, 7, 8, 10, 12, 16, 24, 32, 40, 48, 56, 64, 72, 84 and 96 hours after surgery) and the time to first use of rescue analgesia were similar between treatment groups, the DMTS group compared with the placebo group showed a statistically significantly lesser frequency of rescue analgesic medication use at all evaluated time intervals and a significantly lower morphine equivalent dose and a significantly lower ibuprofen dose at all time intervals.

An integrated variable incorporating both pain scores (NRSSPI) and rescue analgesic medications (IV morphine, oral oxycodone, oral ibuprofen) was determined. Analysis of the integrated variable over 5 to 6, 7, 8, 10, 12, 16, 24, 32, 40, 48, 56, 64, 72, 84 and 96 hours after surgery showed a statistically significant difference between treatment groups at each time interval. Sensitivity analyses of the integrated variable using LOCF or WOCF imputation for pain assessments collected during the efficacious windows of the rescue medications were performed. The results of these analyses also showed a statistically significant difference between treatment group at each time interval.

There was no apparent sedation effect with DMTS; most subjects in both treatment groups at all sedation assessment time points had a Wilson sedation score of 1 (fully awake and oriented).

The 12 cm² DMTS surprisingly outperformed the 15 cm² DMTS in a number of metrics:
Subjects in the 12 cm² DMTS group requested rescue medications less frequently compared to the placebo group than subjects in the 15 cm² DMTS group compared to the placebo group.
Subjects in the 12 cm² DMTS group waited longer to request oxycodone medication than subjects in the 15 cm² DMTS group.
Subjects in the 12 cm² DMTS group used less rescue opioid compared to the placebo group than subjects in the 15 cm² DMTS group compared to the placebo group.
Subjects in the 12 cm² DMTS group exhibited the trend toward lower adverse effects of several types compared to subjects in the 15 cm² DMTS group.
Subjects in the DMTS groups showed a lack of sedation compared to the placebo group.

### 6. EQUIVALENTS AND INCORPORATION BY REFERENCE

While the provided disclosure has been particularly shown and described with reference to a preferred embodiment and various alternate embodiments, it will be understood by persons skilled in the relevant art that various changes in form and details can be made therein without departing from the spirit and scope of the provided disclosure.

All references, issued patents, and patent applications cited within the body of the instant specification, are hereby incorporated by reference in their entirety, for all purposes. In particular, PCT Patent Application No. PCT/US2014/059057 (filed Oct. 3, 2014), PCT Patent Application No. PCT/US2017/059357 (filed Oct. 31, 2017), U.S. Provisional Patent Application No. 63/580,639 (filed Sep. 5, 2023), and U.S. Provisional Patent Application No. 63/651,245 (filed May 23, 2024) are hereby incorporated by reference in their entirety.

1. A transdermal delivery patch comprising a drug layer affixed to a backing layer, wherein:
the drug layer comprises from 1 mg to 3.5 mg dexmedetomidine and a pressure sensitive adhesive;
the drug layer has an adhesive surface suitable for adhesion to a skin surface; and
the transdermal delivery patch is unitary in structure.

2. The transdermal delivery patch of claim 1, wherein the adhesive surface has a surface area from 5.5 cm² to 20 cm².

3.-11. (canceled)

12. The transdermal delivery patch of claim 1, wherein the drug layer comprises from 2.5 mg to 3.5 mg dexmedetomidine.

13. The transdermal delivery patch of claim 12, wherein the adhesive surface has a surface area from 11 cm² to 13 cm².

14. The transdermal delivery patch of claim 13, wherein the drug layer comprises 2.9 mg dexmedetomidine and the adhesive surface has a surface area of 12 cm².

15.-16. (canceled)

17. The transdermal delivery patch of claim 1, wherein the pressure sensitive adhesive comprises an acrylic polymer, acrylate copolymer, acrylate-vinyl acetate copolymer, polyacrylonitrile, or a combination thereof.

18. The transdermal delivery patch of claim 17, wherein the pressure sensitive adhesive comprises a hydroxyl functionalized acrylate copolymer.

19. The transdermal delivery patch of claim 1, wherein the drug layer further comprises a permeation enhancer.

20. The transdermal delivery patch of claim 19, wherein the permeation enhancer comprises lauryl lactate, oleic acid, or a combination thereof.

21. The transdermal delivery patch of claim 20, wherein the permeation enhancer is lauryl lactate.

22. The transdermal delivery patch of claim 1, wherein the backing layer comprises a film.

23. The transdermal delivery patch of claim 22, wherein the film comprises a polyolefin resin, polyacrylic resin, polyester resin, cellophane, polyvinyl alcohol, ethylene-vinyl alcohol copolymer, polyvinyl chloride, polystyrene, polyurethane, polyacrylonitrile, fluororesin, styrene-isoprene-styrene copolymer, styrene-butadiene rubber, polybutadiene, ethylene-vinyl acetate copolymer, polyamide, polysulfone, or a combination thereof.

24. The transdermal delivery patch of claim 23, wherein the film comprises a polyolefin resin selected from polyethylene and polypropylene.

25. The transdermal delivery patch of claim 24, wherein the film comprises a polyester resin.

26. The transdermal delivery patch of claim 25, wherein the film comprises polyethylene and polyethylene terephthalate.

27. The transdermal delivery patch of claim 1, further comprising a release liner adhered to the adhesive surface of the drug layer.

28. The transdermal delivery patch of claim 27, wherein the release liner comprises a silicon or fluoropolymer coating.

29.-31. (canceled)

32. A transdermal delivery patch comprising a drug layer affixed to a backing layer, wherein:
the drug layer comprises from 2.5 mg to 3.5 mg dexmedetomidine, a pressure sensitive adhesive comprising a hydroxyl functionalized acrylate polymer, and lauryl lactate;
the drug layer has an adhesive surface suitable for adhesion to a skin surface, wherein the adhesive surface has a surface area from 11 cm² to 13 cm²;
the backing layer comprises polyethylene and polyethylene terephthalate; and
the transdermal delivery patch is unitary in structure.

33.-44. (canceled)

45. A transdermal delivery patch system comprising at least two of the transdermal delivery patches of claim 1 adjacent to one another.

46.-47. (canceled)

48. A kit comprising a plurality of transdermal delivery patches as described in claim 1.

49. A method of treating pain in a subject in need thereof, the method comprising applying one or more transdermal delivery patches of claim 1 to the skin surface of the subject and maintaining the one or more transdermal delivery patches or transdermal delivery patch system on the skin surface of the subject for at least 72 hours.

50.-81. (canceled)
